(19) United States
(12) Patent Application Publication
(10) Pub. No.: US 2024/0042070 A1
(43) Pub. Date: Feb. 8, 2024

(54) NANOPARTICLES AND TEMPLATE DIRECTED RIG-I AGONIST PRECURSOR COMPOSITIONS AND USES THEREOF FOR CANCER THERAPY

(71) Applicant: Transcode Therapeutics, Inc., Boston, MA (US)

(72) Inventors: Robert Michael Dudley, Boston, MA (US); Qiyong Peter Liu, Boston, MA (US); Dustan Bonnin, Boston, MA (US); Subrata Kumar Ghosh, Boston, MA (US)

(21) Appl. No.: 18/215,550

(22) Filed: Jun. 28, 2023

Related U.S. Application Data
(60) Provisional application No. 63/356,449, filed on Jun. 28, 2022.

(51) Int. Cl. A61K 51/12; A61K 51/04; A61K 49/00; A61K 47/38; A61K 47/36; A61P 35/00

(52) U.S. Cl. CPC A61K 51/1251; A61K 51/0491; A61K 49/0093; A61K 49/006; A61K 47/38; A61K 47/36; A61P 35/00

(57) ABSTRACT

Described herein are compositions and methods for treating cancer using a RIG-I agonist precursor comprising single-stranded 5′ uncapped triphosphate or biphosphate antisense oligonucleotide having a sequence complementary to an endogenous miRNA; optionally wherein the compositions and methods comprise a nanoparticle for targeted delivery of the RIG-I agonist precursor and a radiolabel to a tumor micro environment.

### RELATED APPLICATIONS

This application claims priority to U.S. provisional application No. 63/356,449 filed 28 Jun. 2022 (now expired), which is hereby incorporated into this application in its entirety.

### SEQUENCE LISTING

[001.1] The instant application contains a Sequence Listing which has been submitted electronically in XML format and is hereby incorporated by reference in its entirety. Said XML copy, created on Oct. 10, 2023, is named 1892708-0002-004-101_SL.xml and is 44,734 bytes in size.

### FIELD OF THE DISCLOSURE

This disclosure relates to compositions, including nanoparticles, that comprise a RIG-I agonist precursor comprising a single-stranded 5′ uncapped triphosphate antisense oligonucleotide having a sequence complementary to an endogenous miRNA, formulations for slowing growth of tumors and methods of treating cancer having solid tumors.

### BACKGROUND OF THE DISCLOSURE

Cancer represents a continuing and significant threat to global human health. Harnessing novel mechanisms for treating cancers represents a promising means of delivering therapeutics that meet the ongoing and urgent need for effective cancer treatment. Recent studies have shown that systemic delivery of a synthetic RIG-I (retinoic acid-inducible gene I) agonist inhibits tumor growth. RIG-I senses short double-stranded RNAs with an uncapped 5′-triphosphate moiety, a common motif typically found in viral RNAs. RIG-I is expressed in numerous cell types, including tumor cells, and serves as a promising target for cancer therapy. It is therefore the object of the present disclosure to provide compositions and methods for selectively activating RIG-I using a template directed RIG-I agonist precursor in a tumor microenvironment in order to treat cancers wherein a radiolabeled nanoparticle can further treat and/or diagnose the cancer. A therapeutic methodology harnessing endogenous miRNAs as a means for activating RIG-I provides a highly promising approach to target the tumor microenvironment and treat various associated cancers.

The compositions and methods of the present disclosure provide methods for selectively activating RIG-I in a solid tumor microenvironment utilizing a formulation or composition comprising an effective amount of a RIG-1 agonist precursor comprising a single-stranded 5′ uncapped triphosphate or biphosphate antisense oligonucleotide having a sequence complementary to an endogenous miRNA in combination with core-shell nanoparticles for delivery of both the RIG-I agonist precursor and a radiolabel targeting tumor microenvironments; a targeted two prong approach to treating cancer. Radiolabeled nanoparticles delivering the RIG-I agonist precursors of this disclosure that are targeted to the tumor microenvironment reduces off target toxicity and enhances the efficacy of activating RIG-I. Using radiolabeled nanoparticles comprising the present RIG-agonist precursors with imaging methods provides critical information on the pharmacokinetics and pharmacodynamics of therapeutic agents with direct relevance to the optimization of the dose and dosing schedule, real-time tumor quantitation, tumor heterogeneity, and dynamic tumor changes. All of these parameters are critical in predicting treatment responses and identifying patients who are most likely to benefit from treatment. The present compositions and methods address these problems in current treatment of cancer with solid tumors.

### SUMMARY OF THE INVENTION

In certain embodiments, the application is directed to compositions and uses of RIG-I agonist precursors comprising a single-stranded 5′ uncapped triphosphate antisense oligonucleotide having a sequence complementary to an endogenous miRNA. The RIG-I agonist precursors of the disclosure form a RIG-I agonist in situ/in vivo when an endogenous miRNA complementary sequence is present forming a duplex and activating RIG-I. The RIG-I agonist precursors bind and hybridize with a miRNA that is highly expressed in a solid tumor or solid tumor microenvironment in comparison to a non-solid tumor or non-solid tumor microenvironment.

In certain embodiments the RIG-I agonist precursors are attached (covalently or non-covalently) to a nanoparticle having a core-shell structure. In certain advantageous embodiments, the nanoparticles further comprise a radiolabel. In certain embodiments, the activation of RIG-I may be further enhanced with co-administration (before, after or simultaneously) of a single stranded oligonucleotide sequence complementary to the single-stranded 5′ uncapped triphosphate antisense oligonucleotide (e.g., an endogenous miRNA mimic sequence). In embodiments, the single stranded oligonucleotide sequence complementary to the single-stranded 5′ uncapped triphosphate antisense oligonucleotide is also attached to the nanoparticle. In embodiments, the miRNA is selected from the group consisting of miR10b, miR17, miR18a, miR18b, miR19b, miR21, miR26a, miR29a, miR92a-1, miR92a-2, miR155, miR210, and miR221. In embodiments, the antisense oligonucleotide is selected from SEQ ID NO: 1 to 13.

In certain embodiments, the nanoparticles comprise a core-shell structure wherein the shell comprises a polysaccharide, such as those selected from the group consisting of dextran, alginate, chitosan, chitin, cellulose, hyaluronic acid (HA), amylose, amylopectin, carrageenan, and a polysaccharide polymer consisting of maltotriose units (Pullulan). In embodiments, the shell comprises an aminated polysaccharide coating. In exemplified embodiments, the polysaccharide comprises dextran. In certain embodiments, the dextran comprises a thiolated dextran, a phosphorylated dextran or a dextran sulfate. In certain embodiments, the core comprises a polymer, a metal or a metal ion, wherein the metal or metal oxide is selected from gold, iron, iron oxide, gold alloy, silver, zinc oxide, silicon dioxide (silica), platinum, copper, cobalt, indium, nickel, manganese oxide, calcium carbonate, calcium phosphate or a combination thereof, wherein the polymer is selected from sodium alginate, poly(lactic-co-glycolic) acid (PLGA) polymer, PLGA co-polymer, polysaccharide, chitosan, polystyrene, polycaprolactone, or polyethylene glycol. In exemplified embodiments, the core comprises iron oxide.

In advantageous embodiments, the nanoparticle comprises a radiolabel. In certain embodiments, the radiolabel is selected from an alpha emitter, a beta emitter or a gamma emitter. In embodiments, the radiolabel is selected from copper-64 (Cu-64), copper-67 (Cu-67), F-18, yttrium-90 (Y-90), scandium-44 (SC-44), cobalt-55 (co-55), niobium-90 (Nb-90), rhenium-186 (Re-186), rhenium-188 (Re-188), terbium-161 (Tb-161), lutetium-177 (Lu-177), bismuth-231 (Bi-213), lead-212 (Pb-212), actinium-225 (Ac-225), zirconium-89 (Zr), or any combination thereof. In certain embodiments, the radiolabel comprises copper-64 (Cu-64).

In certain embodiments, the nanoparticle comprises a chelator that is covalently linked to the nanoparticle core and to the radiolabel, and wherein the chelator is covalently linked to the nanoparticle core through a chemical moiety. In embodiments, the chelator comprises DOTA, DOTA-GA, p-SCN-Bn-DOTA, CB-TE2A, CB-TE1A1P, AAZTA, MeCOSar, p-SCN-Bn-NOTA, NOTA, HBED-CC, THP, MASS, DFO, or any combination thereof. In certain embodiments, the chelator comprises 1,4,7-triazacyclononane, 1-glutaric acid-4,7-acetic acid (NODAGA).

In embodiments provided herein are pharmaceutical formulations for slowing growth of tumors in a subject comprising an effective amount the nanoparticles of the disclosure comprising a RIG-I agonist precursor comprising a single-stranded 5′ uncapped triphosphate antisense oligonucleotide having a sequence complementary to an endogenous miRNA. In embodiments, the pharmaceutical formulation is formulated into a dosage form that is an injectable, a tablet, a lyophilized powder, a suspension, or any combination thereof.

In embodiments provided herein are pharmaceutical formulations for slowing growth of tumors in a subject comprising an effective amount of a RIG-I agonist precursor comprising a single-stranded 5′ uncapped triphosphate antisense oligonucleotide having a sequence complementary to an endogenous miRNA. In embodiments, the formulation further comprises a single stranded oligonucleotide sequence complementary to the single-stranded 5′ uncapped triphosphate antisense oligonucleotide.

Provided herein are methods for slowing growth of tumors in a subject in need thereof comprising administering the pharmaceutical formulation of the disclosure or nanoparticle compositions of the disclosure. Provided in other embodiments of the disclosure are methods for treatment of tumors in a subject in need thereof comprising administering the pharmaceutical formulation of the disclosure or nanoparticle compositions of the disclosure. In embodiments, the tumor is a primary tumor. In other embodiments, the tumor is a secondary tumor. In certain embodiments, administering the pharmaceutical formulation or nanoparticle composition induces a rapid and prolonged immune response against the tumor. A prolonged immune response generally being characterized as one lasting longer than about a week (e.g., 7 days) following treatment and associated with acquired or memory immunity against the tumor. A rapid immune response generally being characterized as one that is induced within 24 hours, or within 1-2 days, following treatment and associated with the innate immune system.

In embodiments, the tumor is selected from the group consisting of sarcomas and carcinomas. In certain embodiments, the tumor is selected from the group consisting of bladder cancer, blood cancer, bone cancer, brain cancer, breast cancer, cervical cancer, colon cancer, kidney cancer, esophageal cancer, endometrial cancer, gastric cancer, glioblastoma, cancer of the head and neck, liver cancer, lung cancer, ovarian cancer, pancreatic cancer, prostate cancer, rectal cancer, renal cancer, skin cancer, stomach cancer, thyroid cancer, and uterine cancer. The RIG-I agonist precursors of the disclosure are not antigen specific, but rather activate RIG-I in the tumor microenvironment when a complementary miRNA sequence is present. In other words, the methods of the disclosure can be used in a wide array of tumor types to non-specifically activate the innate immune system and induce immunologic memory to a specific tumor (e.g. acquired immunity to a tumor antigen). In embodiments, the RIG-I activation elicits a tumor-specific immune response. In certain embodiments, the tumor-specific immune response comprises release of type I IFNs, DAMPs (danger-associated molecular patterns), and/or tumor antigens. In embodiments, the nanoparticle radiosensitizes the solid tumor. In embodiments, an effective amount of a RIG-I agonist precursor is an amount sufficient to decrease cancer cell invasion or metastasis in the subject. In certain embodiments, the cancer cell metastasis is from a primary solid tumor to a lymph node in the subject or is from a lymph node to a secondary tissue in a subject.

In certain embodiments, treatment with the RIG-I agonist precursors is a monotherapy. In embodiments, the method further comprises administering additional supportive or adjunctive therapy. In embodiments, the adjunctive therapy comprises radiotherapy, cryotherapy, or ultrasound therapy; the additional supportive or adjunctive therapy comprises a miRNA complementary to the RIG-I agonist precursor; and/or the additional therapeutic agent is selected from the group consisting of a targeted therapy, chemotherapeutic agent, immunotherapeutic agent, an immunogenic cell death inducer (ICDi), and an siRNA therapy. In embodiments, the chemotherapeutic agent is selected from the group consisting of cyclophosphamide, mechlorethamine, chlorambucil, melphalan, daunorubicin, doxorubicin, epirubicin, idarubicin, mitoxantrone, valrubicin, paclitaxel, docetaxel, etoposide, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, vinorelbine, and bevacizumab. In certain embodiments, the targeted therapy is selected from the group consisting of trastuzumab, gilotrif, proleukin, alectinib, campath, atezolizumab, avelumab, axitinib, belimumab, belinostat, bevacizumab, velcade, canakinumab, ceritinib, cetuximab, crizotinib, dabrafenib, daratumumab, dasatinib, denosumab, elotuzumab, enasidenib, erlotinib, gefitinib, ibrutinib, zydelig, imatinib, lenvatinib, midostaurin, necitumumab, niraparib, obinutuzumab, osimertinib, panitumumab, regorafenib, rituximab, ruxolitinib, sorafenib, tocilizumab, and trastuzumab. In certain embodiments, the immunotherapeutic agent is an immune checkpoint inhibitor optionally selected from the group consisting of pembrolizumab (Keytruda®), nivolumab (Opdivo®), atezolizumab (Tecentriq®), ipilimumab (Yervoy®), avelumab (Bavencio®) and durvalumab (Imfinzi®). In embodiments, the supportive or adjunctive therapy is administered prior, concurrently, or after administration of the modified RNA oligonucleotide.

In certain embodiments provided herein are nanoparticles comprising a nanoparticle core that may be radiolabeled and that comprises a single-stranded 5′ uncapped triphosphate or biphosphate RNA oligonucleotide, where the oligonucleotide is complementary to a miRNA, and which is highly expressed in a solid tumor or solid tumor microenvironment in comparison to a non-solid tumor or non-solid tumor microenvironment. The radiolabel may be an alpha emitter, a beta emitter or a gamma emitter. In certain embodiments the radiolabel may have dual energy properties. In further embodiments the radiolabel may be copper-64 (Cu-64), copper-67 (Cu-67), F-18, yttrium-90 (Y-90), scandium-44 (SC-44), cobalt-55 (co-55), niobium-90 (Nb-90), rhenium-186 (Re-186), rhenium-188 (Re-188), terbium-161 (Tb-161), lutetium-177 (Lu-177), bismuth-231 (Bi-213), lead-212 (Pb-212), actinium-225 (Ac-225), zirconium-89 (Zr), or any combination thereof. The radiolabel may be linked to the nanoparticle via a chelator that is covalently linked to the nanoparticle core and to the radiolabel. The chelator may be covalently linked to the nanoparticle core through a chemical moiety that is a secondary amine. In certain aspects, the chelator comprises 1,4,7-triazacyclononane,1-glutaric acid-4,7-acetic acid (NODAGA), DOTA, DOTA-GA, p-SCN-Bn-DOTA, CB-TE2A, CB-TE1A1P, AAZTA, MeCOSar, p-SCN-Bn-NOTA, NOTA, HBED-CC, THP, MASS, DFO, or any combination thereof. In certain embodiments, the nanoparticle core comprises an iron oxide core and may further comprise a polymer coating, such as for example, dextran. The nanoparticle core has a diameter between about 10 nanometers (nm) to about 30 nm and is typically magnetic.

In other aspects, the nanoparticle comprises a RNA oligonucleotide that is covalently linked to the nanoparticle core through a chemical moiety comprising a disulfide bond. The modified RNA oligonucleotide is capable of forming a duplex with an miRNA. The miRNA is selected from the group consisting of miR10b, miR17, miR18a, miR18b, miR19b, miR21, miR26a, miR29a, miR92a-1, miR92a-2, miR155, miR210, and miR221. In certain embodiments, the miRNA is an oncogenic miRNA.

In embodiments provided herein are methods for generating a localized immune response comprising administering to the subject a therapeutically effective amount of a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment thereby generating a localized immune response. In other embodiments provided herein are methods for treating a solid tumor in a subject, the method comprising administering to the subject a therapeutically effective amount of a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment thereby generating a localized immune response. In certain embodiments are also provided methods for detecting, diagnosing, and/or monitoring treatment of a solid tumor in a subject comprising administering the radiolabeled nanoparticle of the disclosure comprising a RIG-1 agonist precursor comprising a single-stranded 5′ uncapped triphosphate antisense oligonucleotide having a sequence complementary to an endogenous miRNA.

In embodiments provided herein are methods for preparing the radiolabeled nanoparticle of the disclosure comprising a present RIG-I agonist precursor, the method comprising preparing the nanoparticle core; covalently linking the modified RIG-I agonist precursor to the nanoparticle core; covalently linking the chelator to the nanoparticle core by reacting the nanoparticle core with the chelator at a ratio of about 40 chelator equivalents per nanoparticle core; adding a solution of ₆₄CuCh to the nanoparticle core; and purifying a mixture of the solution of ₆₄CuCh and the nanoparticle core to yield the nanoparticle. In embodiments the nanoparticle comprises a polysaccharide shell. In certain embodiments, the polysaccharide is aminated and used to attach (covalently or non-covalently) the RIG-I agonist precursor oligonucleotides of the disclosure. In exemplary embodiments, the nanoparticles are prepared with an iron oxide core and a dextran shell.

### BRIEF DESCRIPTION OF THE DRAWINGS

FIG. 1 shows working models for the design and mechanism of action of the template-specific RIG-I agonist precursor as a schematic illustration of the delivery of a 5′triphosphorylated antisense tsRNA, delivered to tumors and metastases. The antisense tsRNA (exemplified as RIGA-miR-21) and tumor specific tsRNA through hybridization produce a 5′ppp-dsRNA, a potent RIG-I agonist (“FIG. 1a”). FIG. 1a discloses SEQ ID NOS 19 and 48, respectively, in order of appearance. Activation of the RIG-I signaling pathway leads to a type I IFN-driven immune response specific to the tumor microenvironment (“FIG. 1c”). This immune response is characterized by activation of dendritic cells (DCs), natural killer cells (NKs), and macrophages (“FIG. 1d”). This process is accompanied by effective tumor antigen presentation by the activated DCs and macrophages and T cell maturation, activation and tumor cell killing. Concomitantly, regulatory T cells (Tregs) are inhibited reducing their immunosuppressive action against the anti-tumor immune response. Importantly, a memory T cell subpopulation is generated that triggers complete immune rejection of the tumor as foreign upon rechallenge. Combined, these processes lead to full remission and resistance to cancer recurrence.

FIG. 2 shows validation of RIG-I activation by RIGA-miR-21 in HEK-Lucia™ RIG-I reporter cells. FIG. 2a shows relative expression of miR-21 in HEK293 reporter cell lines analyzed by RT-PCR; FIG. 2b shows analysis of RIG-I in HEK-Lucia RIG-I cells by Western blotting; FIG. 2c shows activation of RIG-I in reporter cell lines by ppp-dsRNA; FIG. 2d shows activation of RIG-I in reporter cell lines by RIGA-miR-21 (antisense sequence for endogenous miR-21 with 5′-ppp modification) and FIG. 2e shows activation of RIG-I by anti-miR-21 (antisense sequence for endogenous miR-21) and RIGA-miR-21 in the presence of miR-21 mimic (endogenous miR-21 sequence and complementary sequence to RIGA-miR-21 sequence and anti-miR-21 sequence). See Example 3.

FIG. 3 shows validation of RIG-I activation by RIGA-miR-21 in B16-F10 melanoma cells. FIG. 3a shows relative expression of miR-21 in B16-F10; FIG. 3b shows IP-10 induction by RIGA-miR-21 alone or in the presence of miR-21 mimic; FIG. 3c shows caspase 3/7 activation in B16-F10 cells by RIGA-miR-21 or antisense-miR-21; FIG. 3d shows caspase 3/7 activation in B16-F10 cells by RIGA-miR-21 in the presence of miR-21 mimic; FIG. 3e shows Western blot analysis of RIG-I expression induced by RIGA-miR-21 alone or in combination with miR-21 mimic; and, FIG. 3f shows Western blot analysis of the phosphorylation of NF-kB (p65). See Example 4.

FIG. 4 shows, in certain embodiments, nanoparticle formulation, delivery and release of RIGA-miR-21 or antisense-miR-21, wherein dextran coated iron oxide (“ION”) nanoparticles are aminated (“TTX-NH₂”), a reactive linker added (“TTX-PDP”) and a RIG-I agonist precursor oligonucleotide of the disclosure is covalently linked to the nanoparticle (“TTX-Oligo”) and following delivery in vivo the disulfide bond is cleaved releasing the oligonucleotide wherein it binds to an endogenous miRNA forming a RIG-I agonist.

FIG. 5 shows evaluation of RIG-I activation by TTX-RIGA-miR-21 (e.g., RIG-I agonist precursor comprising a sequence complementary to endogenous miR-21) in HEK-Lucia RIG-I reporter cells. FIG. 5a shows RIG-I activation in HEK293 reporter cells induced by TTX-RIGA-miR-21; FIG. 5b shows RIG-I activation in HEK293 reporter cells induced by different concentrations of TTX-RIGA-miR-21 and TTX-antisense-miR-21; FIG. 5c shows relative expression of RIG-I in HEK293 reporter cells induced by TTX-RIGA-miR-21 alone or in combination with miR-21 mimic (endogenous miR-21 sequence); and, FIG. 5d shows relative expression of miR-21 in HEK293 reporter cells induced by TTX-RIGA-miR-21 alone or in combination with miR-21 mimic. See Example 6.

FIG. 6 shows IFN-β and IP-10activation in B16-F10 melanoma cells mediated by TTX-RIGA-miR-21 (+/− miR-21 mimic). FIG. 6a shows relative expression of IFN-r3 mRNA in B16-F10 cells mediated by TTX-RIGA-miR-21 alone or in combination with miR-21 mimic, and control ppp-dsRNA; FIG. 6b shows expression of IFN-r3 protein in B16-F10 cells mediated by TTX-RIGA-miR-21 alone or in combination with miR-21 mimic, and ppp-dsRNA control (RIG-I agonist), FIG. 6c shows relative expression of IP-10 mRNA in HEK293 reporter cells induced by different concentrations of TTX-RIGA-miR-21 and TTX-miR-21, and ppp-dsRNA control; and, FIG. 6d shows expression of IP-10 protein in HEK293 reporter cells induced by different concentrations of TTX-RIGA-miR-21 and TTX-miR-21, and ppp-dsRNA control. See Example 7.

FIG. 7 shows upregulation of RIG-I expression in B16-F10 melanoma cells induced by TTX-RIGA-miR-21 in combination with miR-21 mimic. FIG. 7a shows relative expression of RIG-I in B16-F10 analyzed by RT-PCR; and FIG. 7b shows Western blot analysis of RIG-I protein expression mediated by RIGA-miR-21 alone or in combination with miR-21 mimic.

FIG. 8 shows evaluation of apoptosis in B16-F10 melanoma cells induced by TTX-RIGA-miR-21. FIG. 8a shows caspase 3/7 activation in B16-F10 cells by TTX-RIGA-miR-21 alone or in combination with miR-21; FIG. 8b shows relative expression of TRAIL in B16-F10 cells by RIGA-miR-21 alone or in combination with miR-21 mimic; and, FIG. 8c shows viability of B16-F10 cells mediated by TTX-RIGA-miR-21 alone or in combination with miR-21 mimic. See Example 7.

FIG. 9 shows evaluation of TTX-RIGA-miR-21 in a rodent animal model. FIG. 9a shows reduced tumor growth following intravenous administration of TTX-RIGA-miR-21 and intratumoral administration of control ppp-dsRNA up to 10 days post treatment; FIG. 9b shows tumor weight following treatment with TTX-RIGA-miR-21 compared to control ppp-dsRNA; and, FIG. 9c shows reduced tumor growth of secondary tumors following intravenous administration of TTX-RIGA-miR-21 and intratumoral administration of control ppp-dsRNA wherein TTX-RIGA-miR-21 demonstrated a remarkable capability to inhibit tumor growth on days 21 and 22, while the response to 5′ppp-dsRNA remained no difference from the PBS control. See Example 8.

FIG. 10 illustrates the synthesis of copper-labeled TTX-oligo, wherein dextran coated iron oxide nanoparticles are aminated (“TTX-NH₂”), a chelator of this disclosure added (“TTX-NODAGA”), a reactive linker added (“TTX-NODAGA-PDP”) and a RIG-I agonist precursor oligonucleotide of the disclosure is covalently linked to the nanoparticle (“TTX-Oligo-NODAGA”) and finally a radiolabel of this disclosure added to the chelator (“TTX-Oligo-^{nat/64}Cu”) forming a radiolabel nanoparticle RIG-I agonist precursor of this disclosure. See Example 9.

FIG. 11 shows radioiodine isotopes most commonly used in imaging and therapy (FIG. 11a) and reagents for iodine radiolabeling of proteins (FIG. 1ib). See Example 10.

FIG. 12 illustrates the synthesis of iodine radio-labeled TTX-oligo, wherein dextran coated iron oxide nanoparticles are aminated (“TTX-NH₂”), a radiolabel precursor added (“TTX-HPP”), an iodine radiolabel added (“TTX-*I”), a reactive linker added (“TTX-*I-PDP”) and a RIG-I agonist precursor oligonucleotide of the disclosure is covalently linked to the nanoparticle (“TTX-Oligo-*I”) forming an iodine radiolabel nanoparticle RIG-I agonist precursor of this disclosure. See Example 10.

### DETAILED DESCRIPTION OF THE DISCLOSURE

### Introduction

This disclosure relates to the use of a RIG-I agonist precursor comprising a single-stranded 5′ uncapped triphosphate or biphosphate antisense oligonucleotide (“RIG-I agonist precursor”) that acts in a template directed manner having a sequence complementary to an endogenous (tumor) miRNA to form a RIG-I agonist in vivo/in situ. Applicants demonstrated that the present RIG-I agonist precursors activate RIG-I in cancer cells and when administered to a solid tumor rodent model rapidly, following administration, slowed the growth of both primary and secondary tumors while inducing a prolonged immune response against the tumors. See Example 7 and 8. Accordingly, provided herein are compositions comprising the present RIG-I agonist precursors and their method of use for treating and/or slowing the growth of tumors (e.g., solid tumors).

In certain embodiments provided herein is a pharmaceutical formulation for slowing growth of tumors in a subject comprising an effective amount of a RIG-1 agonist precursor comprising a single-stranded 5′ uncapped triphosphate antisense oligonucleotide having a sequence complementary to an endogenous miRNA. In certain other embodiments provided herein are nanoparticles comprising a core-shell structure, wherein the shell comprises a RIG-I agonist precursor comprising a single-stranded 5′ uncapped triphosphate antisense oligonucleotide having a sequence complementary to an endogenous miRNA. In certain other embodiments provided herein are nanoparticles, formulations and methods thereof, wherein the nanoparticles comprise a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment.

### Overview

miRNAs in Cancer

Small RNAs, such as miRNAs, exert their regulatory functions from within ribonucleoprotein complexes termed RISCs (RNA-induced silencing complexes). The core subunit of RISC is a small RNA bound to a member of the Argonaute family of proteins. Argonaute uses the small RNA as a guide to identify complementary target transcripts for silencing through a variety of mechanisms. MiRNAs are generally captured by the human Argonaute 2 protein (AGO2) and are capable of regulating gene expression by base-pairing to complementary mRNA targets while associated with AGO2. The miRNA captured by AGO2 serves as a guide RNA to accept and hybridize with complementary RNA targets, forming a double-stranded RNA duplex. It has been shown that highly complementary RNA targets facilitate release of the guide RNA:target RNA duplex from AGO2.

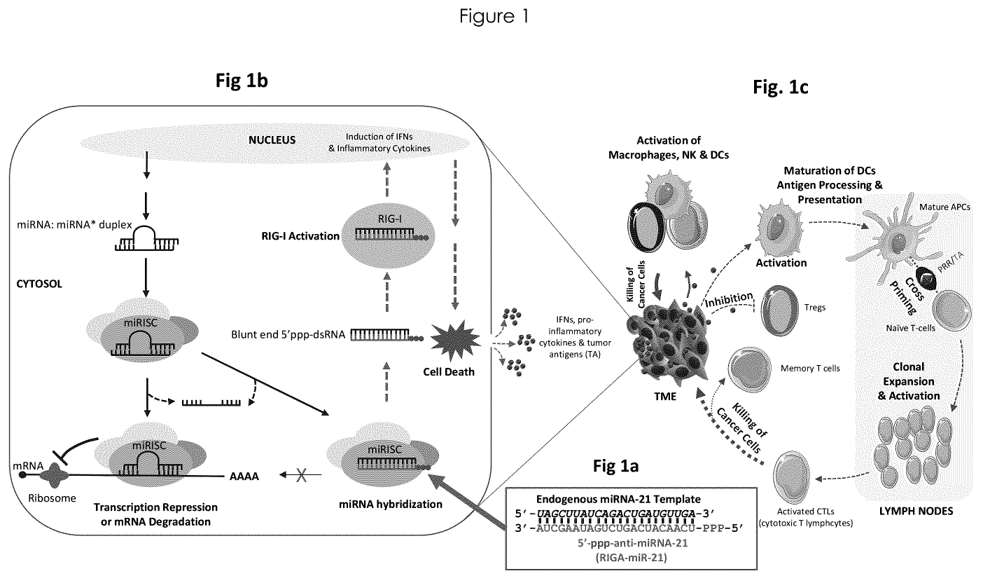

Retinoic acid-inducible gene I (RIG-I)-like receptors (RLRs) are key RNA sensors, mediating the transcriptional induction of type I interferons and other genes that collectively establish an antiviral host response (Yong H Y, Luo D. 2018; 9:1379). RIG-I is expressed in virtually all cell types, including tumor cells, and is a promising alternative to enhance ICI (immune checkpoint inhibitors) efficacy (Heidegger S. et al., 2019. EBioMedicine. 41:146. Poeck H., et al. 2008. Nat. Med. 14:1256). Preclinical studies have shown that systemic delivery of a synthetic RIG-I agonist inhibits tumor growth through mechanisms similar to those triggered for elimination of virally-infected cells (Poeck H., et al. 2008. 5′-triphosphate-siRNA: turning gene silencing and Rig-I activation against melanoma. Nat. Med. 14:1256). RIG-I engagement leads to preferential tumor cell death (via intrinsic or extrinsic apoptosis, and inflammasome-induced pyroptosis), and to IFN-I-mediated activation of the innate and adaptive immune systems (see FIG. 1 of Elion DL., et al. 2018. Oncotarget. 9:29007). RGT100, a specific RIG-I agonist, recently reported results from two phase I studies as a monotherapy (NCT03065023) and in combination with pembrolizumab (NCT03739138) concluding patients experienced tolerable safety but that only a modest antitumor activity was demonstrated at the doses tested for RGT100 [Moreno., et al. 2022. Cancer Immunol Immunother; 71(12):2985-2998].

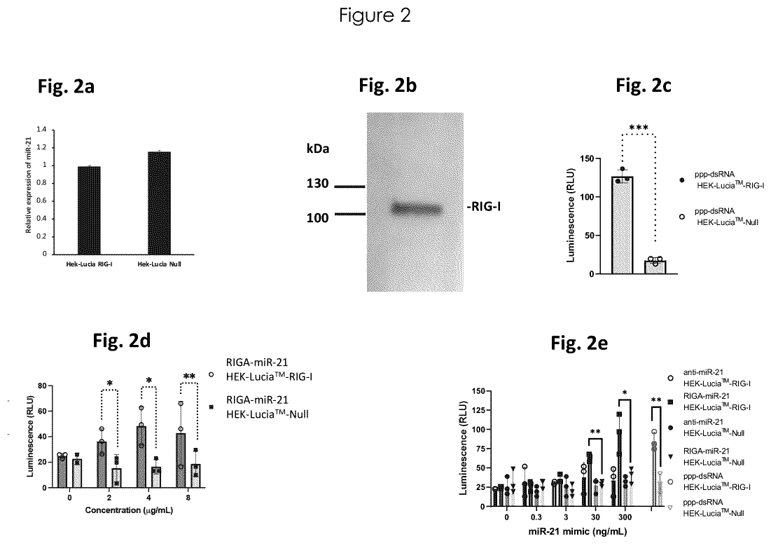

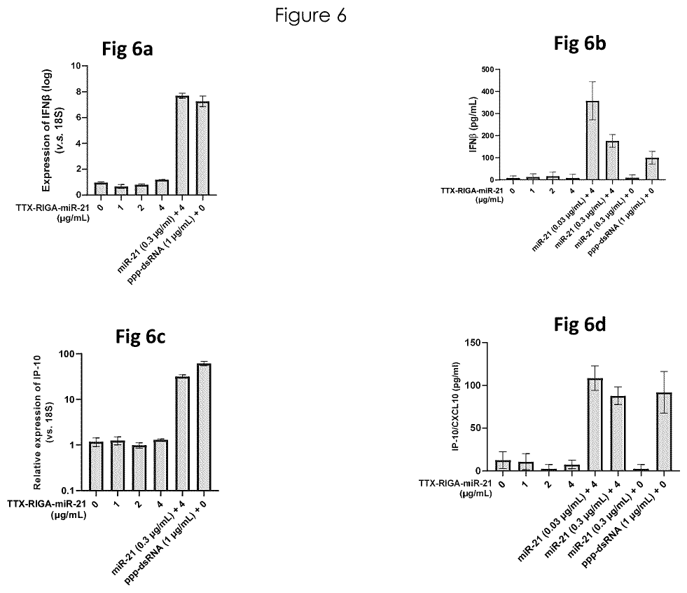

Without being bound by theory, the RIG-I pathway may be selectively activated in cancer cells according to the methods and compositions of the present disclosure, by in situ generation of 5′ppp-dsRNA following introduction of 5′ppp RNA complementary to a miRNA (5′ppp anti-miRNA) or mRNA expressed specifically in these cells (FIG. 1). Applicant demonstrated activation of RIG-I in HEK-Lucia RIG-I reporter cells using ss-ppp-miRNA-21 as the RIG-I agonist precursor as well as in melanoma cells. See FIG. 2e and Example 3 and 6-8 and Example 7. The same or similar selective activation of the RIG-I pathway is expected from 5′pp-dsRNA. Consequently, the antitumor immunity potential of the tumor microenvironment (TME) can be uncovered via the activation of RIG-I signaling pathway, in conjunction with concurrent activation of certain tumor suppressor gene(s), by simply using a single-stranded RNA. See FIG. 6.

The utility of the RIG-I agonist triphosphate RNA for melanoma therapy has been recently validated (Helms M W. et al. 2019. Utility of the RIG-I Agonist Triphosphate RNA for Melanoma Therapy. Mol Cancer Ther. 2019; 18(12):2343-2356). It is also noted that the similarity of RIG-I's natural ligand, triphosphate RNA (5′ppp-dsRNA) (and 5′pp) to small interfering RNA (siRNA) has led to the development of bifunctional siRNAs for concurrent silencing of oncogenic or immunosuppressive targets and activation of the RIG-I signaling pathway (Poeck H., et al. 2008. Nat. Med. 14:1256. Ellermeier J. et al. 2013. 2013; 73(6):1709-1720). The combined approaches mount a two-targeted attack on the tumor cells with encouraging outcomes.

MicroRNAs (miRNAs) are small non-coding RNAs that can regulate various target genes. miRNAs regulate gene expression at the post-transcriptional level through base-pairing with complementary sequences of messenger RNAs (mRNA). This interaction results in gene silencing by cleavage of the mRNA strand, destabilization of the mRNA through shortening of its polyA tail, or inhibition of translation of the mRNA into proteins. miRNAs control the expression of approximately 60% of protein-coding genes and regulate cell metabolism, proliferation, differentiation, and apoptosis (Huang Z, Shi J, Gao Y, et al. HMDD v3.0: a database for experimentally supported human microRNA-disease associations. Nucleic Acids Res. 2019; 47 (D1):D1013-D1017).

Under normal physiological conditions, miRNAs function in feedback mechanisms by safeguarding key biological processes including cell proliferation, differentiation and apoptosis (Reddy, K. B., Cancer Cell International, 2015, 15:38). miRNAs are expressed in a wide variety of organs and cells, and regulate both pro- and anti-inflammatory actions. miRNAs have emerged as key regulators of the inflammatory response in a wide spectrum of human disease (Tahamtan, A., et al., Front Immunol. 2018; 9: 1377).

Dysregulation of miRNA expression has been linked to a variety of disease indications such as cancer. More than 50% of miRNA genes were revealed to be located in cancer-associated genomic regions (Di Leva, G., et al., Annu Rev Pathol. 2014; 9:287-314). The dysregulation of miRNAs has been shown to perform a fundamental role in the onset, progression and dissemination of numerous types of cancer. For example, miRNA dysregulation is known to be associated with chronic lymphocytic leukemia, where miR-15a and miR-16-1 were shown to be downregulated or deleted in the majority of patients with chronic lymphocytic leukemia (Calin G. A., et al., Proc Natl Acad Sci USA; 2002; pp. 15524-15529). Other miRNAs, such as miR-21, miR-26, and miR-29a, have been shown to be preferentially expressed in cancer cells and/or the tumor cell microenvironment (Chakraborty, C., et al., Mol Ther Nucleic Acids. 2020 Jun. 5; 20: 606-620). A therapeutic methodology directed against endogenous miRNAs therefore provides a highly promising approach to target the tumor microenvironment and treat various cancers associated with dysregulated miRNAs.

RIG-I Mediated RNA Induced Immunogenic Cell Death

The pattern recognition receptor, Retinoic acid-inducible gene I (RIG-I), recognizes specific molecular patterns of viral RNAs for inducing type I interferon. RIG-I consists of two N-terminal caspase recruitment domains (CARDs), a central RNA helicase domain, and a C-terminal RNA-binding domain. The C-terminal domain (CTD) of RIG-I recognizes the 5′-ppp group of non-self RNAs and undergoes a conformational change to induce IFN-r3 production (Lee, M., et al., Nucleic Acids Research, 2016, Vol. 44, No. 17). Structural and biochemical studies have demonstrated that RIG-I CTD can bind to blunt-ended dsRNAs containing a 5′-ppp. Studies have shown that 5′-ppp dsRNA strongly binds to the RIG-I CTD and stimulates interferon production more effectively compared to 5′-OH dsRNA (Pichlmair, A., et al., 2006, Science, 314, 997-1001; Vela, A., et al., 2012, J. Biol. Chem., 287, 42564-42573).

RIG-I-like receptor ligands have been used as a promising strategy for the treatment of solid malignancies including melanoma, pancreatic cancer and breast cancer in preclinical models. The major features of RIG-I are its ubiquitous expression and signaling outcomes, notably, type I IFN production and preferential tumor cell death, which are two keys factors in potent T cell responses. Despite the potential success of the RIG-I approach, the immune system is powerful and incompletely understood, warranting cautious optimism and thorough examination of the caveats associated with innate immune activation, including possible on-target induction of autoimmunity, or induction of a cytokine ‘storm’ which could pose a threat to patient safety. It is important to note that, since RIG-I is expressed in most cells of the human body, the consequences of RIG-I activation might be widespread, driving symptoms like fatigue, depression and cognitive impairment.

In certain embodiments, the present disclosure presents a strategy to mitigate the potential side effects associated with RIG-I therapy by restricting RIG-I activation to the tumor microenvironment. Specifically, tumor-specific miRNAs are used as templates for the assembly of 5′ppp-dsRNA RIG-I agonists. To accomplish this, the present methods introduce exogenously supplied 5′ppp single-stranded oligonucleotide (“RIG-I agonist precursor”) (e.g., RNA) that is complementary to the endogenous miRNA. The complementary miRNA (endogenous) and single stranded 5′ppp oligonucleotide (RIG-I agonist precursor) hybridize and form a 5′ppp-dsRNA (“RIG-I agonist”) that promotes release from AGO2. The released 5′ppp-dsRNA RIG-I agonist facilitates potent activation of RIG-I signaling. Through this process of using a template directed RIG-I agonist precursor, the RIG-I activation will be limited to cancer cells, essentially eliminating nonspecific immune system activation elsewhere in the body. An additional level of specificity can be achieved by coupling the exogenous single-stranded 5′ppp oligonucleotide to a nanoparticle carrier that preferentially localizes to the tumor microenvironment. As shown in FIG. 1, substitution of the 5(p)pp-anti-mRNA or-miRNA approaches described herein for standard RNAi technology for silencing target miRNA or mRNAs can promote RIG-I activation that triggers RIG-I signaling and cell death, thereby improving treatment outcomes. In vivo, 5′(p)pp-anti-mRNA/miRNA can hybridize with and silence the target mRNA or miRNA, resulting in the formation of 5′(p)pp-ds-mRNAs/-miRNAs that bind to and activate RIG-I proteins, leading to RIG-I signaling and cancer cell death.

Applicant has herein demonstrated that the RIG-I agonist precursors of this disclosure, activate RIG-I in melanoma cells by forming a RIG-I agonist in situ, and that treatment with the present RIG-I agonist precursor, when delivered using nanoparticles, slow the growth of primary and secondary tumors. The present compositions, and methods thereof provide treatments for cancer with solid tumors and which can be used to prevent second tumor formation via immunological cell mediated memory against the tumor. See Example 8 and 9.

Treatment and Diagnosis of Cancer Using Radiation-Induced miRNA Expression

Radiation is used in approximately 50% of all cancer treatments with an aim to maximize damage to the tumor while minimizing damage to the surrounding healthy tissue. Ionizing radiation damages cells primarily by inducing ionization and DNA damage. Gholami Y H, et al. Sci Rep. 2019; 9(1):14346. This DNA damage may directly or indirectly trigger alterations in the expression levels of miRNAs. Sriharshan A. J Proteomics Bioinform. 2014; 07 (10). Czochor J R, et al. 2014; 21(2):293-312. Radiation-induced miRNA expression may provide a survival response in cancer cells that impedes therapies targeting the cancer.

In certain embodiments, the present disclosure presents a strategy to use radiation-induced miRNA expression to treat cancers by targeting the tumor-specific miRNAs and using targeted radiation to increase miRNA expression in cells that have internalized a radiolabeled nanoparticle. To accomplish this, the present methods introduce a nanoparticle comprising a nanoparticle core, a radiolabel, and a single-stranded 5′ uncapped triphosphate or biphosphate modified RNA oligonucleotide (also referred to herein as a “modified RNA oligonucleotide”). The modified RNA oligonucleotide is linked to the nanoparticle core, and it is complementary to a miRNA which is highly expressed in a solid tumor or solid tumor microenvironment in comparison to a non-solid tumor or non-solid tumor microenvironment. As described previously the modified RNA oligonucleotide hybridizes with the endogenous miRNA and forms a 5′ppp double stranded RNA that activates RIG-I signaling. This process limits RIG-I activation to cancer cells, essentially eliminating nonspecific immune system activation elsewhere in the body.

For instance, miR10b may be a good target for the treatment of metastatic tumors as miR10b is upregulated in many tumors which have invasive and metastatic properties. Kim J, et al. Cancer Research. 2016; 76(21):6424-6435. However, not all metastasis will necessarily express miR10b or they may express miR10b at lower levels. In such instances, administration of a radiolabeled nanoparticle may be used to induce miRNA expression (e.g., the expression of miR10b) in a localized manner. Specifically, the radiolabeled nanoparticle comprising a modified RNA oligonucleotide may be internalized by a cancer cell in a subject. The subject can then be imaged to determine the location or number of cancer cells, and radiation can then be administered to the subject in a localized manner. While some cancer cells that have internalized the nanoparticle may already express the miR10b target prior to radiation, radiation may increase miR10b expression across targeted cancer cells that already express or do not express miR10b. As such, provided herein is a method of treating cancer by combining RIG-I mediated immune activation against tumor cells while, optionally, inhibiting a miRNA or mRNA (e.g., use of a modified RNA oligonucleotide which is complementary to endogenous miR21).

In addition to targeting cancer cells through RIG-I activation, the radiolabeled nanoparticles of the present application can also locally enhance radiation-induced death of cancer cells. Specifically, in conventional external beam radiotherapy, the dose of radiation that is administered to a cancer cell is frequently limited by the presence of critical organs near the area of treatment. Gholami Y H, et al. Sci Rep. 2019; 9(1):14346. However, the radiation of nanoparticles using external beam radiotherapy or internal radionuclide therapy has been shown to enhance the radiation dose to the localized cancer. Id. As such, radiation of the localized nanoparticles may radiosensitize the cancer by amplifying the effects of radiation within the tumor cells.

Additionally, the nanoparticles described herein can be used detect, diagnose, and/or monitor treatment of cancer in the subject. Accordingly, the present application provides a new class of theranostic nanoparticles that enable imaging contrast while simultaneously amplifying radiation dose under clinical irradiation conditions and activating RIG-I resulting in cell death, and thereby improving treatment outcomes.

### Definitions

The terms used in this specification generally have their ordinary meanings in the art, within the context of this disclosure and in the specific context where each term is used. Certain terms are discussed below or elsewhere in the specification, to provide additional guidance to the practitioner in describing the compositions and methods of the disclosure and how to make and use them. The scope or meaning of any use of a term will be apparent from the specific context in which the term is used.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values.

Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Numeric ranges disclosed herein are inclusive of the numbers defining the ranges.

By the term “nucleic acid” is meant any single- or double-stranded polynucleotide (e.g., DNA or RNA, cDNA, semi-synthetic, or synthetic origin). The term nucleic acid includes oligonucleotides containing at least one modified nucleotide (e.g., containing a modification in the base and/or a modification in the sugar) and/or a modification in the phosphodiester bond linking two nucleotides. In some embodiments, the nucleic acid can contain at least one modified ribose such as a 2′-fluoro (2′-F). In some embodiments, the nucleic acid can contain a 5′ uncapped triphosphate or biphosphate. Non-limiting examples of nucleic acids are described herein. Additional examples of nucleic acids are known in the art.

A nucleic acid disclosed herein can comprise an oligonucleotide sequence which is not naturally occurring. Such variants necessarily have less than 100% sequence identity or similarity with the starting molecule. In certain embodiments, the variant will have a nucleic acid sequence from about 75% to less than 100% amino acid sequence identity or similarity with the nucleic acid sequence of the starting (e.g., naturally-occurring or wild-type) oligonucleotide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule. In certain aspects, the oligonucleotide sequence will be fully complementary to a target sequence. In other words, the duplex region formed by the oligonucleotide and its target will exhibit a fully complementary sequence (i.e., does not comprise any base pair mismatches or gaps) without taking into account in overhang. In certain aspects, the oligonucleotide and the target sequence does not comprise more than 0-5 base pair mismatches in the duplex region.

Tumor-specific RNAs of the present disclosure can comprise a microRNA (miRNA) or messenger RNA (mRNA). miRNAs or mRNAs of the present disclosure may comprise oncogenic miRNAs or mRNAs. Oncogenic miRNAs or mRNAs are miRNAs or mRNAs that are believed to be involved in or associated with a tumor/tumors and/or cancer.

The term “diamagnetic” is used to describe a composition that has a relative magnetic permeability that is less than or equal to 1 and that is repelled by a magnetic field.

The term “highly expressed” refers to a state wherein there exists any measurable increase in expression over normal or baseline levels. For example, a molecule (e.g., miRNA) that is overexpressed in a cancer is one that is manifest in a measurably higher level in the presence of the cancer than in the absence of the cancer. Such an increase can be at least two-fold at least three-fold, or more. In certain embodiments, a molecule (e.g., miRNA) that is overexpressed in a cancer is one that is manifest in a measurably higher level, such as an increase of at least 10%, 15%, 20%, 25%, 30%, 40% or 50% or more in the presence of the cancer than in the absence of the cancer.

The term “paramagnetic” is used to describe a composition that develops a magnetic moment only in the presence of an externally applied magnetic field.

The term “ferromagnetic” is used to describe a composition that is strongly susceptible to magnetic fields and is capable of retaining magnetic properties (a magnetic moment) after an externally applied magnetic field has been removed.

By the term “nanoparticle” is meant an object that has a diameter between about 2 nm to about 200 nm (e.g., between 10 nm and 200 nm, between 2 nm and 100 nm, between 2 nm and 40 nm, between 2 nm and 30 nm, between 2 nm and 20 nm, between 2 nm and 15 nm, between 100 nm and 200 nm, and between 150 nm and 200 nm). Non-limiting examples of nanoparticles include the nanoparticles described herein.

By the term “magnetic nanoparticle” is meant a nanoparticle (e.g., any of the nanoparticles described herein) that is magnetic (as defined herein). Non-limiting examples of magnetic nanoparticles are described herein. Additional magnetic nanoparticles are known in the art.

The terms “subject” or “patient,” as used herein, refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

As used herein, the term “tumor” refers to an abnormal mass of tissue and/or cells in which the growth of the mass surpasses, and is not coordinated with, the growth of normal tissue, including both solid masses (e.g., as in a solid tumor) or fluid masses (e.g., as in a hematological cancer) or any cancer cell found within the tumor. A tumor can be solid (e.g., lymphoma, sarcoma or carcinoma) or non-solid (e.g., tumors of the blood, bone marrow, or lymph nodes such as leukemia). A tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue.

The term “microenvironment” as used herein means any portion or region of a tissue or body that has constant or temporal, physical or chemical differences from other regions of the tissue or regions of the body.

The term “tumor microenvironment” as used herein refers to the environment in which a tumor exists, which is the non-cellular area within the tumor and the area directly outside the tumorous tissue but does not pertain to the intracellular compartment of the cancer cell itself. It also refers cells found within the tumor microenvironment, e.g., fibroblasts, endothelial cells, adipocytes, pericytes, neuroendocrine cells, or immune cells in tumor microenvironment (macrophage, B cells, T cells etc.) The tumor and the tumor microenvironment are closely related and interact constantly. A tumor can change its microenvironment, and the microenvironment can affect how a tumor grows and spreads. Typically, the tumor microenvironment has a low pH in the range of 5.0 to 7.0, or in the range of 5.0 to 6.8, or in the range of 5.8 to 6.8, or in the range of 6.2-6.8. On the other hand, a normal physiological pH is in the range of 7.2-7.8. The tumor microenvironment is also known to have lower concentration of glucose and other nutrients, but higher concentration of lactic acid, in comparison with blood plasma. Furthermore, the tumor microenvironment can have a temperature that is 0.3 to 1° C. higher than the normal physiological temperature.

The term “non-tumor microenvironment” refers to a microenvironment at a site other than a tumor.

The term “metastasis” refers to the migration of a cancer cell present in a primary tumor to a secondary, non-adjacent tissue in a subject. Non-limiting examples of metastasis include: metastasis from a primary tumor to a lymph node (e.g., a regional lymph node), bone tissue, lung tissue, liver tissue, and/or brain tissue. The term metastasis also includes the migration of a metastatic cancer cell found in a lymph node to a secondary tissue (e.g., bone tissue, liver tissue, or brain tissue). In some non-limiting embodiments, the cancer cell present in a primary tumor is a breast cancer cell, a colon cancer cell, a kidney cancer cell, a lung cancer cell, a skin cancer cell, an ovarian cancer cell, a pancreatic cancer cell, a prostate cancer cell, a rectal cancer cell, a stomach cancer cell, a thyroid cancer cell, or a uterine cancer cell. Additional aspects and examples of metastasis are known in the art or described herein.

The term “primary tumor” refers to a tumor present at the anatomical site where tumor progression began and proceeded to yield a cancerous mass. In some embodiments, a physician may not be able to clearly identify the site of the primary tumor in a subject.

The term “metastatic tumor” refers to a tumor in a subject that originated from a tumor cell that metastasized from a primary tumor in the subject. In some embodiments, a physician may not be able to clearly identify the site of the primary tumor in a subject.

Preferred methods and materials are described herein, although methods and materials similar or equivalent to those described herein can also be used in the practice or testing of the presently disclosed methods and compositions. All publications, patent applications, patents, and other references mentioned herein are incorporated by reference in their entirety.

### Endogenous Tumor-Specific RNAs

Described herein are compositions and methods for eliciting an immune response through compositions comprising oligonucleotides complementary to endogenous tumor-specific RNAs. In some embodiments, the disclosure provides a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a tumor or tumor microenvironment in comparison to a non-tumor or non-tumor microenvironment.

In some embodiments, the disclosure provides a method of generating a localized immune response comprising administering to the subject a therapeutically effective amount of a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a tumor or tumor microenvironment in comparison to a non-tumor or non-tumor microenvironment thereby generating a localized immune response.

In some embodiments, the disclosure provides a method of treating cancer in a subject, the method comprising administering to the subject a therapeutically effective amount of a nanoparticle comprising a nanoparticle core; a radiolabel; and a single-stranded uncapped triphosphate or biphosphate modified RNA oligonucleotide that is linked to the nanoparticle core, wherein said oligonucleotide is complementary to a miRNA which is highly expressed in a tumor or tumor microenvironment in comparison to a non-tumor or non-tumor microenvironment thereby generating a localized immune response.

In some embodiments, the disclosure provides a method of detecting, diagnosing, and/or monitoring treatment of cancer in a subject, the method comprising administering to the subject a nanoparticle comprising a nanoparticle core; a radiolabel; and a single-stranded uncapped triphosphate or biphosphate modified RNA oligonucleotide that is linked to the nanoparticle core, wherein said oligonucleotide is complementary to a miRNA which is highly expressed in a tumor or tumor microenvironment in comparison to a non-tumor or non-tumor microenvironment thereby generating a localized immune response.

In some embodiments, the single-stranded 5′ uncapped triphosphate or biphosphate modified RNA oligonucleotide comprises a sequence which is complementary to an endogenous tumor-specific RNA, which tumor specific RNA is specific to a tumor cell, wherein RIG-I is selectively activated in tumor cells highly expressing the tumor-specific RNA. Endogenous tumor-specific RNAs of the present disclosure may be selected from an miRNA or mRNA. Endogenous tumor-specific RNAs of the present disclosure may be further selected from an oncogenic miRNA or oncogenic mRNA. Oncogenic miRNAs or mRNAs are miRNAs or mRNAs that are believed to be involved in cancer.

MiRNAs have been shown to be a component in many cancers and may provide novel avenues for cancer treatment. MiRNAs of the methods and compositions of the present disclosure include but are not limited to: miR-9; miR-10b; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. A complete list, including sequences, is available at the OncomiRDB (Wang et al. Bioinformatics. 2014; 30(15):2237-2238; mircancer.ecu.edu/browse.jsp; US20150004221A1); see also Table 1 and Table 2).

An example of one such a miRNA is miR-10b. Upregulation of miR-10b has been shown to be responsible for migration and invasion of metastatic tumor cells as well as the viability of these cells (Tian Y., et al., J. Biol. Chem. 2010; 285:7986-7994). Analysis of miR-10b levels in 40 human esophageal cancer samples and their paired normal adjacent tissues revealed an elevated expression of miR-10b in 95% (38 of 40) of the sampled cancer tissues (Tian Y., et al., J. Biol. Chem. 2010; 285:7986-7994). There are many other miRNAs that also play a role in carcinogenesis that represent relevant targets; these and other miRNAs represent a potential new class of targets for therapeutic inhibition (Nguyen D D, Chang S. Int J Mol Sci. 2017; 19(1):65). For example, miR-21 has been shown to be involved in a variety of cancer cells and tissues, not limited to glioblastoma, breast, colorectal, lung, pancreas, skin, liver, gastric, cervical, and thyroid cancers, as well as various lymphatic, and hematopoietic cancers and neuroblastoma. miR-21 is a representative example of a single miRNA that targets multiple oncogenic signaling cascades and causes global dysregulation of gene expression networks in cancer cells (Pan, X., et al., Cancer Biol. Ther. 2010; 10:1224-1232). Increased miR-21 expression has been found to target a variety of essential tumor suppressors such as phosphatase and tensin homolog (PTEN), PDCD4, RECK, TPM1, facilitating cell proliferation, survival, metastasis, and the acquisition of a chemoresistant phenotype (Meng, F., et al., Gastroenterology. 2007; 133:647-658; Peralta-Zaragoza O., et al., BMC Cancer. 2016; 16:215; Zhang, X., et al., BMC Cancer. 2016; 16:86; Reis S T., et al., BMC Urol. 2012; 12:14; Zhu S., et al., J. Biol. Chem. 2007; 282:14328-14336).

MiR-155 is epigenetically controlled by BRCA1, and is overexpressed in breast, ovarian, and lung cancers. miR-155 has been investigated as a potential biomarker for B-cell cancers. Overexpression of MiR-155 blocks B-cell differentiation via downregulation of the SHIP1 and C/EBPβ genes, and results in improved cell survival due to the activation of PI3K-Akt and MAPK pathways. In other cancers, such as glioma, overexpression of miR-155 promotes the progression of tumor formation through negative correlation with caudal-type homeobox 1 protein (CDX1) expression in glioma tissue.

MiR-210 is a well-documented miRNA implicated in various aspects of cancer development, progression and metastasis. Increased miR-210 expression was observed in bone metastatic and non-bone metastatic prostate cancer tissue. Expression was found to be elevated in bone metastatic prostate cancer tissue relative to non-bone metastatic prostate cancer tissue, and was shown to promote prostate cancer cell epithelial-mesenchymal transition and bone metastasis via the NF-κB signaling pathway (Ren D., et al., Mol Cancer. 2017; 16: 117). Other miRNAs, such as miRNA-221, have been found to be upregulated in breast cancer, glioma, hepatocellular carcinoma, pancreatic adenocarcinoma, melanoma, chronic lymphocytic leukemia, and thyroid papillary carcinoma (Brognara E., et al., Int J Oncol. 2012 December; 41(6):2119-27).

In some embodiments, the disclosure provides a method of treating cancer in a subject, the method comprising administering to the subject a therapeutically effective amount of a nanoparticle comprising a nanoparticle core; a radiolabel; and a single-stranded uncapped triphosphate or biphosphate modified RNA oligonucleotide that is linked to the nanoparticle core, wherein said oligonucleotide is complementary to a miRNA which is highly expressed in a tumor or tumor microenvironment in comparison to a non-tumor or non-tumor microenvironment thereby generating a localized immune response. In some embodiments, the endogenous tumor-specific RNA is an oncogenic miRNA. In some embodiments, the endogenous tumor-specific RNA is not an oncogenic miRNA.

In some embodiments, the endogenous tumor-specific RNA is a miRNA selected from the group consisting of miR-9; miR-10b; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. In some embodiments, the endogenous tumor-specific RNA is miR-9. In some embodiments, the endogenous tumor-specific RNA is miR-10b. In some embodiments, the endogenous tumor-specific RNA is miR-17. In some embodiments, the endogenous tumor-specific RNA is miR-18. In some embodiments, the endogenous tumor-specific RNA is miR-19b. In some embodiments, the endogenous tumor-specific RNA is miR-21. In some embodiments, the endogenous tumor-specific RNA is miR-26a. In some embodiments, the endogenous tumor-specific RNA is miR-29a. In some embodiments, the endogenous tumor-specific RNA is miR-92a. In some embodiments, the endogenous tumor-specific RNA is miR-106b/93. In some embodiments, the endogenous tumor-specific RNA is miR-125b. In some embodiments, the endogenous tumor-specific RNA is miR-130a. In some embodiments, the endogenous tumor-specific RNA is miR-155. In some embodiments, the endogenous tumor-specific RNA is miR-181a. In some embodiments, the endogenous tumor-specific RNA is miR-200s. In some embodiments, the endogenous tumor-specific RNA is miR-210. In some embodiments, the endogenous tumor-specific RNA is miR-210-3p. In some embodiments, the endogenous tumor-specific RNA is miR-221. In some embodiments, the endogenous tumor-specific RNA is miR-222. In some embodiments, the endogenous tumor-specific RNA is miR-221/222. In some embodiments, the endogenous tumor-specific RNA is miR-335. In some embodiments, the endogenous tumor-specific RNA is miR-498. In some embodiments, the endogenous tumor-specific RNA is miR-504. In some embodiments, the endogenous tumor-specific RNA is miR-1810. In some embodiments, the endogenous tumor-specific RNA is miR-1908. In some embodiments, the endogenous tumor-specific RNA is miR-224/452. In some embodiments, the endogenous tumor-specific RNA is miR-181/340.

In a preferred embodiment of the present disclosure, the endogenous tumor-specific RNA is selected from the group consisting of: miR10b, miR17, miR18a, miR18b, miR19b, miR21, miR26a, miR29a, miR92a-1, miR92a-2, miR155, miR210, and miR221. In some embodiments, the endogenous tumor-specific RNA is miR10b. In some embodiments, the endogenous tumor-specific RNA is miR17. In some embodiments, the endogenous tumor-specific RNA is miR18a. In some embodiments, the endogenous tumor-specific RNA is miR18b. In some embodiments, the endogenous tumor-specific RNA is miR19b. In some embodiments, the endogenous tumor-specific RNA is miR21. In some embodiments, the endogenous tumor-specific RNA is miR26a. In some embodiments, the endogenous tumor-specific RNA is miR29a. In some embodiments, the endogenous tumor-specific RNA is miR92a-1. In some embodiments, the endogenous tumor-specific RNA is miR92a-2. In some embodiments, the endogenous tumor-specific RNA is miR155. In some embodiments, the endogenous tumor-specific RNA is miR210. In some embodiments, the endogenous tumor-specific RNA is miR22.

In some embodiments, the endogenous tumor-specific RNA which is highly expressed in tumor cells is selected from the group consisting of: miR-9; miR-10b; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. In some embodiments, the tumor cell is associated with bone and non-bone metastatic cancers, breast cancer, glioma, hepatocellular carcinoma, pancreatic adenocarcinoma, melanoma, thyroid papillary carcinoma, glioblastoma, colorectal cancer, lung cancer, kidney cancer, pancreatic cancer, skin cancer, liver cancer, gastric cancer, cervical cancer, thyroid cancers, lymphatic cancers, hematopoietic cancers, neuroblastoma, esophageal cancer, osteosarcoma, ovarian cancer, oral cancer, bladder cancer, adenoid cystic carcinoma, anaplastic thyroid carcinoma, astrocytoma, meningioma, retinoblastoma.

In some embodiments, the tumor cell is associated with bone metastatic cancer. In some embodiments, the tumor cell is associated with non-bone metastatic cancer. In some embodiments, the tumor cell is associated with breast cancer. In some embodiments, the tumor cell is associated with glioma. In some embodiments, the tumor cell is associated with hepatocellular carcinoma. In some embodiments, the tumor cell is associated with pancreatic adenocarcinoma. In some embodiments, the tumor cell is associated with melanoma. In some embodiments, the tumor cell is associated with thyroid papillary carcinoma. In some embodiments, the tumor cell is associated with glioblastoma. In some embodiments, the tumor cell is associated with colorectal cancer. In some embodiments, the tumor cell is associated with lung cancer. In some embodiments, the tumor cell is associated with kidney cancer. In some embodiments, the tumor cell is associated with pancreatic cancer. In some embodiments, the tumor cell is associated with skin cancer. In some embodiments, the tumor cell is associated with liver cancer. In some embodiments, the tumor cell is associated with gastric cancer. In some embodiments, the tumor cell is associated with cervical cancer. In some embodiments, the tumor cell is associated with thyroid cancer. In some embodiments, the tumor cell is associated with lymphatic cancers. In some embodiments, the tumor cell is associated with hematopoietic cancers. In some embodiments, the tumor cell is associated with neuroblastoma. In some embodiments, the tumor cell is associated with esophageal cancer. In some embodiments, the tumor cell is associated with osteosarcoma. In some embodiments, the tumor cell is associated with ovarian cancer. In some embodiments, the tumor cell is associated with oral cancer. In some embodiments, the tumor cell is associated with bladder cancer. In some embodiments, the tumor cell is associated with adenoid cystic carcinoma. In some embodiments, the tumor cell is associated with anaplastic thyroid carcinoma. In some embodiments, the tumor cell is associated with astrocytoma. In some embodiments, the tumor cell is associated with meningioma. In some embodiments, the tumor cell is associated with retinoblastoma.

Many web-based tools for identifying microRNAs involved in human cancer are available. For a review, see Mar-Aguilar F, Rodríguez-Padilla C, Reśendez-Pérez D. Web-based tools for microRNAs involved in human cancer, Oncol Lett. 2016; 11(6):3563-3570. The databases can be mined for miRNAs associated with a particular type of cancer, or the behavior of a particular miRNA in different malignancies at the same time, and the sequences of a specific miRNA can be readily retrieved from various databases. For example, miRCancer (mircancer.ecu.edu) is a database that stores records of miRNA and cancer associations collected through data mining. A rule-based approach was devised to analyze the title and abstract of 26,414 publications (2016) and to find full sentences or phrases that included the names of the miRNA and the cancer type, and any expression terms. The results of this data mining process were then corroborated by hand. miRCancer has records of >3,764 miRNA-cancer associations from 2,611 publications, which amounts to 236 miRNA expression profiles from 176 human cancers. miRCancer is freely accessible online, and the database can be searched by miRNA name or cancer type, or a combination of both (Xie B, Ding Q, Han H, Wu D. miRCancer: a microRNA-cancer association database constructed by text mining on literature. Bioinformatics. 2013; 29(5):638-644).

As an example, mining of the database (Dec. 16, 2020) found miR-10b to be upregulated in 20 types of cancers including acute myeloid leukemia, bladder cancer, colorectal cancer, endometrial cancer, esophageal cancer, esophageal squamous cell carcinoma, gastric cancer, gastric cancer, glioblastoma, glioma, hepatocellular carcinoma, lung cancer, malignant melanoma, medulloblastoma, nasopharyngeal carcinoma, non-small cell lung cancer, oral cancer, osteosarcoma, pancreatic cancer, and pancreatic ductal adenocarcinoma. Similarly, over 100 miRNAs including miR-10b were found to be related to breast cancer including hsa-miR-101, hsa-miR-106a, hsa-miR-106b, hsa-miR-10b, hsa-miR-1207-5p, hsa-miR-1228, hsa-miR-1229, hsa-miR-1246, hsa-miR-125a, hsa-miR-125b, hsa-miR-1307-3p, hsa-miR-135a, hsa-miR-140, hsa-miR-141, hsa-miR-150, hsa-miR-150-5p, hsa-miR-153, hsa-miR-155, hsa-miR-17, hsa-miR-17-5p, hsa-miR-181a, hsa-miR-181b, hsa-miR-181b-3p, hsa-miR-182, hsa-miR-182-5p, hsa-miR-183, hsa-miR-183-5p, hsa-miR-18a, hsa-miR-18b, hsa-miR-191, hsa-miR-1915-3p, hsa-miR-196a, hsa-miR-197, hsa-miR-19a, hsa-miR-19b, hsa-miR-200a, hsa-miR-200a-3p, hsa-miR-200b, hsa-miR-200c, hsa-miR-203, hsa-miR-205, hsa-miR-205-5p, hsa-miR-206, hsa-miR-20a, hsa-miR-20b, hsa-miR-21, hsa-miR-214-3p, hsa-miR-217, hsa-miR-221, hsa-miR-222, hsa-miR-223, hsa-miR-224, hsa-miR-224-5p, hsa-miR-23a, hsa-miR-23b, hsa-miR-24, hsa-miR-24-2-5p, hsa-miR-24-3p, hsa-miR-27a, hsa-miR-27b, hsa-miR-29a, hsa-miR-301a-3p, hsa-miR-3136-3p, hsa-miR-3188, hsa-miR-32, hsa-miR-330-3p, hsa-miR-346, hsa-miR-3646, hsa-miR-370, hsa-miR-372, hsa-miR-372-3p, hsa-miR-373, hsa-miR-374a, hsa-miR-376b, hsa-miR-378, hsa-miR-423, hsa-miR-429, hsa-miR-4469, hsa-miR-449a, hsa-miR-4513, hsa-miR-4530, hsa-miR-4732-5p, hsa-miR-494, hsa-miR-495, hsa-miR-498, hsa-miR-5003-3p, hsa-miR-503, hsa-miR-503-3p, hsa-miR-510, hsa-miR-520c, hsa-miR-520e, hsa-miR-520g, hsa-miR-526b, hsa-miR-544a, hsa-miR-645, hsa-miR-655, hsa-miR-660-5p, hsa-miR-665, hsa-miR-675, hsa-miR-761, hsa-miR-762, hsa-miR-9, hsa-miR-92a, hsa-miR-92a-3p, hsa-miR-93, hsa-miR-93-5p, hsa-miR-937, hsa-miR-944, hsa-miR-96, and hsa-miR-96-5p.

The sequence of miR-10b or any sequence of interest can be retrieved from miRbase, the microRNA database (mirbase.org/):

>hsa-miR-10b-5p MIMAT0000254
(SEQ ID NO: 14)
UACCCUGUAGAACCGAAUUUGUG
>hsa-miR-10b-3p MIMAT0004556
(SEQ ID NO: 46)
ACAGAUUCGAUUCUAGGGGAAU

TABLE 1
Sequences of upregulated miRNAs associated with certain
cancers

| microRNA | | | Cancer (partial |
|---|---|---|---|
| ID | Accession # | Sequence (5′-3′) | list) |
| hsa-miR-9- | MIMAT0000441 | UCUUUGGUUAUCUAGCUGUA | Breast, cervical, |
| 5p | | UGA (SEQ ID NO: 27) | and glioma |
| | | | cancer |
| hsa-let-7a- | MIMAT0000062 | UGAGGUAGUAGGUUGUAUAG | Acute myeloid |
| 5p | | UU (SEQ ID NO: 28) | leukemia |
| hsa-miR- | MIMAT0000680 | UAAAGUGCUGACAGUGCAGA | Breast cancer, |
| 106b-5p | | U (SEQ ID NO: 29) | cervical, |
| | | | colorectal and |
| | | | gastric cancer |
| hsa-miR- | MIMAT0000254 | UACCCUGUAGAACCGAAUUU | Glioblastoma, |
| 10b-5p | | GUG (SEQ ID NO: 14) | esophageal and |
| | | | breast cancer |
| hsa-miR- | MIMAT0004593 | GCUCUUUUCACAUUGUGCUA | Gastric cancer, |
| 130a-5p | | CU (SEQ ID NO: 30) | cervical and |
| | | | osteosarcoma |
| | | | cancer |
| hsa-miR- | MIMAT0000449 | UGAGAACUGAAUUCCAUGGG | Hepatocellular |
| 146a-5p | | UU (SEQ ID NO: 31) | carcinoma, |
| | | | cervical and |
| | | | colorectal |
| | | | cancer |
| hsa-miR- | MIMAT0000646 | UUAAUGCUAAUCGUGAUAGG | Liver, lung, |
| 155-5p | | GGUU (SEQ ID NO: 24) | kidney, glioma |
| | | | and pancreatic |
| | | | cancer; B cell |
| | | | lymphoma and |
| | | | lymphoid |
| | | | leukemia |
| hsa-miR- | MIMAT0000256 | AACAUUCAACGCUGUCGGUG | Cervical, |
| 181a-5p | | AGU (SEQ ID NO: 32) | Breast, cervical, |
| | | | colon, and |
| | | | gastric cancer |
| hsa-miR- | MIMAT0000257 | AACAUUCAUUGCUGUCGGUG | Breast cancer, |
| 181b-5p | | GGU (SEQ ID NO: 33) | cervical, |
| | | | osteosarcoma, |
| | | | ovarian and |
| | | | prostate cancer |
| hsa-miR- | MIMAT0007881 | CGGCGGGGACGGCGAUUGGU | Glioblastoma, |
| 1908-5p | | C (SEQ ID NO: 34) | osteosarcoma |
| hsa-miR- | MIMAT0001620 | CAUCUUACCGGACAGUGCUG | Breast, ovarian |
| 200a-5p | | GA (SEQ ID NO: 35) | and esophageal |
| | | | cancer |
| hsa-miR- | MIMAT0000267 | CUGUGCGUGUGACAGCGGCU | Prostate cancer |
| 210-3p | | GA (SEQ ID NO: 36) | |
| hsa-miR- | MIMAT0026475 | AGCCCCUGCCCACCGCACACU | Cervical, |
| 210-5p | | G (SEQ ID NO: 25) | colorectal, |
| | | | esophageal, |
| | | | glioma and lung |
| | | | cancer |
| hsa-miR- | MIMAT0004564 | UGCCUGUCUACACUUGCUGU | Oral, gastric and |
| 214-5p | | GC (SEQ ID NO: 37) | pancreatic |
| | | | cancer |
| hsa-miR-21- | MIMAT0000076 | UAGCUUAUCAGACUGAUGUU | Glioblastoma, |
| 5p | | GA (SEQ ID NO: 19) | breast, |
| | | | colorectal, lung, |
| | | | pancreas, liver, |
| | | | gastric, cervical |
| | | | and |
| | | | hematopoietic |
| | | | cancer |
| hsa-miR- | MIMAT0004568 | ACCUGGCAUACAAUGUAGAU | Bladder, breast, |
| 221-5p | | UU (SEQ ID NO: 26) | cervical, colon, |
| | | | gastric and liver |
| | | | cancer |
| hsa-miR- | MIMAT0004569 | CUCAGUAGCCAGUGUAGAUC | Adenoid cystic |
| 222-5p | | CU (SEQ ID NO: 38) | carcinoma, |
| | | | anaplastic |
| | | | thyroid |
| | | | carcinoma, |
| | | | bladder and |
| | | | breast cancer |
| Ihsa-miR-224- | MIMAT0000281 | UCAAGUCACUAGUGGUUCCG | Bladder, breast, |
| 5p | | UUUAG (SEQ ID NO: 39) | cervical, |
| | | | colorectal |
| | | | cancer and |
| | | | Glioblastoma |
| hsa-miR-335- | MIMAT0000765 | UCAAGAGCAAUAACGAAAAA | astrocytoma, |
| 5p | | UGU (SEQ ID NO: 40) | colorectal cancer |
| | | | and |
| | | | meningioma |
| hsa-miR- | MIMAT0004692 | UUAUAAAGCAAUGAGACUGA | Gastric and |
| 340-5p | | UU (SEQ ID NO: 41) | thyroid cancer |
| hsa-miR-452- | MIMAT0001635 | AACUGUUUGCAGAGGAAACU | Hepatocellular |
| 5p | | GA (SEQ ID NO: 42) | carcinoma, |
| | | | colorectal and |
| | | | esophageal |
| | | | cancer |
| hsa-miR-498- | MIMAT0002824 | UUUCAAGCCAGGGGGCGUUU | Breast cancer and |
| 5p | | UUC (SEQ ID NO: 43) | retinoblastoma |
| hsa-miR- | MIMAT0002875 | AGACCCUGGUCUGCACUCUA | Osteosarcoma |
| 504-5p | | UC (SEQ ID NO: 44) | |
| hsa-miR-93- | MIMAT0000093 | CAAAGUGCUGUUCGUGCAGG | Breast, cervical |
| 5p | | UAG (SEQ ID NO: 45) | and lung cancer |

TABLE 2
Select Sequences/reverse
complement sequences for miRNAs

| miRNA | miRNA 5P | Reverse |
|---|---|---|
| (human) | Sequence | Complement |
| miR10b | uacccuguag | cacaaauucg |
| | aaccgaauuu | guucuacagg |
| | gug | gua |
| | (SEQ ID | (SEQ ID |
| | NO: 14) | NO: 1) |
| miR17 | caaagugcuu | cuaccugcac |
| | acagugcagg | uguaagcacu |
| | uag | uug |
| | (SEQ ID | (SEQ ID |
| | NO: 15) | NO: 2) |
| miR18a | uaaggugcau | cuaucugcac |
| | cuagugcaga | uagaugcacc |
| | uag | uua |
| | (SEQ ID | (SEQ ID |
| | NO: 16) | NO: 3) |
| miR18b | uaaggugcau | cuaacugcac |
| | cuagugcagu | uagaugcacc |
| | uag | uua |
| | (SEQ ID | (SEQ ID |
| | NO: 17) | NO: 4) |
| miR19b | aguuuugcag | gcuggaugca |
| | guuugcaucc | aaccugcaaa |
| | agc | acu |
| | (SEQ ID | (SEQ ID |
| | NO: 18) | NO: 5) |
| miR21 | uagcuuauca | ucaacaucag |
| | gacugauguu | ucugauaagc |
| | ga | ua |
| | (SEQ ID | (SEQ ID |
| | NO: 19) | NO: 47) |
| miR26a | uucaaguaaa | agccuauccu |
| | uccaggauag | ggauuuacuu |
| | gcu | gaa |
| | (SEQ ID | (SEQ ID |
| | NO: 20) | NO: 7) |
| miR29a | acugauuucu | cugaacacca |
| | uuugguguuc | aaagaaauca |
| | ag | gu |
| | (SEQ ID | (SEQ ID |
| | NO: 21) | NO: 8) |
| miR92a-1 | agguugggau | agcauugcaa |
| | cgguugcaau | ccgaucccaa |
| | gcu | ccu |
| | (SEQ ID | (SEQ ID |
| | NO: 22) | NO: 9) |
| miR92a-2 | ggguggggau | guaaugcaac |
| | uuguugcauu | aaauccccac |
| | ac | cc |
| | (SEQ ID | (SEQ ID |
| | NO: 23) | NO: 10) |
| miR155 | uuaaugcuaa | aaccccuauc |
| | ucgugauagg | acgauuagca |
| | gguu | uuaa |
| | (SEQ ID | (SEQ ID |
| | NO: 24) | NO: 11) |
| miR210 | agccccugcc | cagugugcgg |
| | caccgcacac | ugggcagggg |
| | ug | cu |
| | (SEQ ID | (SEQ ID |
| | NO: 25) | NO: 12) |
| miR221 | accuggcaua | aaaucuacau |
| | caauguagau | uguaugccag |
| | uu | gu |
| | (SEQ ID | (SEQ ID |
| | NO: 26) | NO: 13) |

The methods and compositions of the present disclosure may be extended to other RNA targets such as mRNAs coding for a protein that promotes cancer development. In some embodiments, the disclosure provides a method for treating cancer comprising administering a single-stranded 5′ uncapped triphosphate or biphosphate modified RNA oligonucleotide, wherein said oligonucleotide is complementary to an endogenous tumor-specific RNA which is highly expressed in tumor cells in comparison to non-tumor cells. In some embodiments, the disclosure provides a method of treating cancer in a subject, the method comprising administering to the subject a therapeutically effective amount of a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a tumor or tumor microenvironment in comparison to a non-tumor or non-tumor microenvironment thereby generating a localized immune response. In some embodiments, the disclosure provides a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a tumor or tumor microenvironment in comparison to a non-tumor or non-tumor microenvironment. In some embodiments, the endogenous tumor-specific RNA is an mRNA. In some embodiments, the endogenous tumor-specific RNA is an mRNA. In some embodiments, the endogenous tumor-specific RNA is not an mRNA.

A number of mRNAs are believed to be involved in cancer. The entire or partial antisense strand of the mRNA including the poly-A tail can be generated from a DNA template by in vitro transcription, and modified with 5′(p)pp. The 5′(p)pp-anti-mRNA sequence can be optimized to contain sequence elements that increase RNA stability. An anti-mRNA may be formulated with lipids to obtain an RNA—lipid nanoparticle drug product. In vivo, 5′(p)pp-anti-mRNA can hybridize with and silence the target mRNA resulting in the formation of 5′(p)pp-ds-mRNAs that bind and activate RIG-I proteins, and lead to RIG-I signaling and cancer cell death. See Table 3 for a list of exemplary mRNA transcripts.

TABLE 3
Select oncogenes and their RefSeq accession numbers

| Oncogene Category | Examples | mRNA Accession # |
|---|---|---|
| Cytoplasmic | Src- | NM_005417.5, NM_198291.3 |
| tyrosine kinases | family | |
| Cytoplasmic | RAF | NM_002880.4 |
| Serine/threonine | kinase | |
| kinases and their | | |
| regulatory subunits | | |
| Regulatory GTPases | RAS | NM_001130442.3, NM_005343.4, |
| | protein | NM_176795.5, NM_001318054.2 |
| | (HRas) | |
| | RAS | NM_001130442.3, NM_033360.4, |
| | protein | NM_001369786.1, NM_001369787.1 |
| | (KRas) | |
| | RAS | NM_002524.5 |
| | protein | |
| | (NRas) | |
| Transcription | MYC | NM_005376.5, NM_001354870.1, |
| factors | gene | NM_005378.6 |
| Inhibitor of | BIRC5 | NM_001012270.2, NM_001012271.2, |
| apoptosis | | NM_001168.3 |
| (IAP) family | | |

For example, Survivin (also named BIRC5), a well-known cancer therapeutic target, can be targeted using this approach. Survivin, a multi-regulator of cell cycle and apoptosis is overexpressed in all human cancers but demonstrates low expression in normal tissues. Its increased expression has been detected in 90% of primary breast cancers and correlates with poor clinical outcomes. Furthermore, increased surviving levels have been shown to be significantly associated with negative hormone receptor status. Importantly, high levels of survivin have been detected in other cancers such as pancreatic cancer, where it correlates with both cellular proliferation and apoptosis pointing to a possible ubiquitous role of this anti-apoptotic marker. Considering the potential value of reducing or abolishing survivin expression as a means of overcoming chemoresistance, the process of RNA interference (RNAi) can prove valuable. Indeed, down-regulation of BIRC5 by RNAi demonstrated promise in acute lymphoblastic leukemia, lung, and cervical carcinoma in vitro and breast cancer in vivo (Ghosh S K, Yigit M V, Uchida M, et al. Sequence-dependent combination therapy with doxorubicin and a survivin-specific small interfering RNA nanodrug demonstrates efficacy in models of adenocarcinoma. Int J Cancer. 2014; 134(7):1758-1766). Sequence-dependent combination therapy with doxorubicin and a survivin-specific small interfering RNA nanodrug demonstrates efficacy in models of adenocarcinoma.

Substitution of the current 5(p)pp-anti-mRNA approach for standard siRNA technology for silencing survivin can promote RIG-I activation that triggers RIG-I signaling and cell death, thereby improving treatment outcome.

### Radiolabeled Nanoparticles Comprising Modified RNA Oligonucleotides

In certain aspects, the disclosure relates to nanoparticles and uses thereof. In certain embodiments, the nanoparticle comprises a nanoparticle core, a radiolabel, and a single-stranded 5′ uncapped triphosphate or biphosphate modified RNA oligonucleotide that is linked to the nanoparticle core, wherein said oligonucleotide is complementary to a miRNA which is highly expressed in a tumor or tumor microenvironment in comparison to a non-tumor or non-tumor microenvironment.

Oligonucleotides and Oligonucleotide Modifications

In certain embodiments, the nanoparticles provided herein comprise a single-stranded 5′ uncapped triphosphate or biphosphate modified RNA oligonucleotide that is linked to the nanoparticle core, wherein said oligonucleotide is complementary to a miRNA which is highly expressed in a tumor or tumor microenvironment in comparison to a non-tumor or non-tumor microenvironment.

Exogenous RNA comprising a 5′ triphosphate (ppp) has been shown to induce an immunogenic form of cell death in different tumor entities (Elion, D L., et al Cancer Res. 2018 Nov. 1; 78(21):6183-6195; Besch, R., et al, J Clin Investig. 2009; 119:2399-411; Duewell, P., et al., Cell Death Differ. 2014; 21:1825-37; Kuber, K., et al., Cancer Res. 2010; 70:5293-304). 5′ biphosphate (5′pp) or 5′ triphosphate modification (5′ppp), may be referred to herein as 5′pp and 5′ppp anti-miRNAs/mRNAs, respectively. 5′-ppp-RNA has been shown to induce cytokine release combined with direct sensing of viral RNA by immune cells, and promote an adaptive cellular immune response directed against tumor cells (Poeck, H., et al., Nat Med. 2008 November; 14(11):1256-63). The pattern recognition receptor, RIG-I, can bind to blunt-ended dsRNAs containing an uncapped 5′ppp or 5′pp. As disclosed herein, uncapped refers to an RNA lacking a 5′ cap structure consisting of a 7-methylguanosine triphosphate linked to the 5′ end of the mRNA via a 5′→5′ triphosphate linkage. The present disclosure provides a method for selectively activating RIG-I in tumor cells comprising administering a single-stranded 5′ uncapped triphosphate or biphosphate modified RNA oligonucleotide, wherein said single-stranded 5′ uncapped triphosphate or biphosphate modified RNA oligonucleotide comprises a sequence, which is complementary to an endogenous tumor-specific RNA. The present disclosure also provides a single-stranded 5′ uncapped triphosphate or biphosphate modified RNA oligonucleotide, wherein said oligonucleotide is complementary to a miRNA, which is highly expressed in tumor tissue in comparison to non-tumor tissue. The 5′triphosphate structure is shown below:

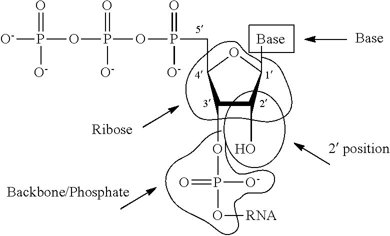

In embodiments, the single-stranded 5′ uncapped triphosphate or biphosphate modified RNA oligonucleotide comprises an uncapped 5′ triphosphate. In embodiments, the single-stranded 5′ uncapped triphosphate or biphosphate modified RNA oligonucleotide comprises an uncapped 5′ biphosphate.

In some embodiments, the single-stranded 5′ uncapped triphosphate or biphosphate modified RNA oligonucleotide comprises a sequence which is complementary to an miRNA. In some embodiments, the single-stranded 5′ uncapped triphosphate or biphosphate modified RNA oligonucleotide comprises a sequence which is complementary to an endogenous miRNA. In some embodiments, the single-stranded 5′ uncapped triphosphate or biphosphate modified RNA oligonucleotide comprises a sequence which is complementary to an oncogenic miRNA selected from the group consisting of: miR-9; miR-10b; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. In some embodiments, the single-stranded 5′ uncapped triphosphate or biphosphate modified RNA oligonucleotide comprises a sequence which is complementary to miR-9. In some embodiments, the single-stranded 5′ uncapped triphosphate or biphosphate modified RNA oligonucleotide comprises a sequence which is complementary to miR-10b. In some embodiments, the single-stranded 5′ uncapped triphosphate or biphosphate modified RNA oligonucleotide comprises a sequence which is complementary to miR-17. In some embodiments, the single-stranded 5′ uncapped triphosphate or biphosphate modified RNA oligonucleotide comprises a sequence which is complementary to miR-18. In some embodiments, the single-stranded 5′ uncapped triphosphate or biphosphate modified RNA oligonucleotide comprises a sequence which is complementary to miR-19b. In some embodiments, the single-stranded 5′ uncapped triphosphate or biphosphate modified RNA oligonucleotide comprises a sequence which is complementary to miR-21. In some embodiments, the single-stranded 5′ uncapped triphosphate or biphosphate modified RNA oligonucleotide comprises a sequence which is complementary to miR-26a. In some embodiments, the single-stranded 5′ uncapped triphosphate or biphosphate modified RNA oligonucleotide comprises a sequence which is complementary to miR-29a. In some embodiments, the single-stranded 5′ uncapped triphosphate or biphosphate modified RNA oligonucleotide comprises a sequence which is complementary to miR-92a. In some embodiments, the single-stranded 5′ uncapped triphosphate or biphosphate modified RNA oligonucleotide comprises a sequence which is complementary to miR-106b/93. In some embodiments, the single-stranded 5′ uncapped triphosphate or biphosphate modified RNA oligonucleotide comprises a sequence which is complementary to miR-125b. In some embodiments, the single-stranded 5′ uncapped triphosphate or biphosphate modified RNA oligonucleotide comprises a sequence which is complementary to miR-130a. In some embodiments, the single-stranded 5′ uncapped triphosphate or biphosphate modified RNA oligonucleotide comprises a sequence which is complementary to miR-155. In some embodiments, the single-stranded 5′ uncapped triphosphate or biphosphate modified RNA oligonucleotide comprises a sequence which is complementary to miR-181a. In some embodiments, the single-stranded 5′ uncapped triphosphate or biphosphate modified RNA oligonucleotide comprises a sequence which is complementary to miR-200s. In some embodiments, the single-stranded 5′ uncapped triphosphate or biphosphate modified RNA oligonucleotide comprises a sequence which is complementary to miR-210. In some embodiments, the single-stranded 5′ uncapped triphosphate or biphosphate modified RNA oligonucleotide comprises a sequence which is complementary to miR-210-3p. In some embodiments, the single-stranded 5′ uncapped triphosphate or biphosphate modified RNA oligonucleotide comprises a sequence which is complementary to miR-221. In some embodiments, the single-stranded 5′ uncapped triphosphate or biphosphate modified RNA oligonucleotide comprises a sequence which is complementary to miR-222. In some embodiments, the single-stranded 5′ uncapped triphosphate or biphosphate modified RNA oligonucleotide comprises a sequence which is complementary to miR-221/222. In some embodiments, the single-stranded 5′ uncapped triphosphate or biphosphate modified RNA oligonucleotide comprises a sequence which is complementary to miR-335. In some embodiments, the single-stranded 5′ uncapped triphosphate or biphosphate modified RNA oligonucleotide comprises a sequence which is complementary to miR-498. In some embodiments, the single-stranded 5′ uncapped triphosphate or biphosphate modified RNA oligonucleotide comprises a sequence which is complementary to miR-504. In some embodiments, the single-stranded 5′ uncapped triphosphate or biphosphate modified RNA oligonucleotide comprises a sequence which is complementary to miR-1810. In some embodiments, the single-stranded 5′ uncapped triphosphate or biphosphate modified RNA oligonucleotide comprises a sequence which is complementary to miR-1908. In some embodiments, the single-stranded 5′ uncapped triphosphate or biphosphate modified RNA oligonucleotide comprises a sequence which is complementary to miR-224/452. In some embodiments, the single-stranded 5′ uncapped triphosphate or biphosphate modified RNA oligonucleotide comprises a sequence which is complementary to miR-181/340.

In certain embodiments of the present disclosure, the single-stranded 5′ uncapped triphosphate or biphosphate modified RNA oligonucleotide comprises a sequence which is complementary to miR10b, miR17, miR18a, miR18b, miR19b, miR21, miR26a, miR29a, miR92a-1, miR92a-2, miR155, miR210, and miR221. In some embodiments, the single-stranded 5′ uncapped triphosphate or biphosphate modified RNA oligonucleotide comprises a sequence which is complementary to miR10b. In some embodiments, the single-stranded 5′ uncapped triphosphate or biphosphate modified RNA oligonucleotide comprises a sequence which is complementary to miR17. In some embodiments, the single-stranded 5′ uncapped triphosphate or biphosphate modified RNA oligonucleotide comprises a sequence which is complementary to miR18a. In some embodiments, the single-stranded 5′ uncapped triphosphate or biphosphate modified RNA oligonucleotide comprises a sequence which is complementary to miR18b. In some embodiments, the single-stranded 5′ uncapped triphosphate or biphosphate modified RNA oligonucleotide comprises a sequence which is complementary to miR19b. In some embodiments, the single-stranded 5′ uncapped triphosphate or biphosphate modified RNA oligonucleotide comprises a sequence which is complementary to miR21. In some embodiments, the single-stranded 5′ uncapped triphosphate or biphosphate modified RNA oligonucleotide comprises a sequence which is complementary to miR26a. In some embodiments, the single-stranded 5′ uncapped triphosphate or biphosphate modified RNA oligonucleotide comprises a sequence which is complementary to miR29a. In some embodiments, the single-stranded 5′ uncapped triphosphate or biphosphate modified RNA oligonucleotide comprises a sequence which is complementary to miR92a-1. In some embodiments, the single-stranded 5′ uncapped triphosphate or biphosphate modified RNA oligonucleotide comprises a sequence which is complementary to miR92a-2. In some embodiments, the single-stranded 5′ uncapped triphosphate or biphosphate modified RNA oligonucleotide comprises a sequence which is complementary to miR155. In some embodiments, the single-stranded 5′ uncapped triphosphate or biphosphate modified RNA oligonucleotide comprises a sequence which is complementary to miR210. In some embodiments, the single-stranded 5′ uncapped triphosphate or biphosphate modified RNA oligonucleotide comprises a sequence which is complementary to miR22.

In some embodiments, the single-stranded 5′ uncapped triphosphate or biphosphate modified RNA oligonucleotide forms a duplex with the miRNA. In a preferred embodiment, the duplex comprises a 5′ blunt end. In some embodiments, the single-stranded 5′ uncapped triphosphate or biphosphate modified RNA oligonucleotide forms a duplex with a miRNA selected from the group consisting of: miR-9; miR-10b; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. In a preferred embodiment, the single-stranded 5′ uncapped triphosphate or biphosphate modified RNA oligonucleotide forms a duplex with a miRNA selected from the group consisting of: miR10b, miR17, miR18a, miR18b, miR19b, miR21, miR26a, miR29a, miR92a-1, miR92a-2, miR155, miR210, and miR221. In some embodiments, the single-stranded 5′ uncapped triphosphate or biphosphate modified RNA oligonucleotide is capable of forming a duplex with said miRNA, wherein the duplexed portion of the oligonucleotide is complementary to at least 10 contiguous nucleotides within a miRNA. In some embodiments, the duplexed portion of the oligonucleotide is complementary to at least 11 contiguous nucleotides within a miRNA. In some embodiments, the duplexed portion of the oligonucleotide is complementary to at least 12 contiguous nucleotides within a miRNA. In some embodiments, the duplexed portion of the oligonucleotide is complementary to at least 13 contiguous nucleotides within a miRNA. In some embodiments, the duplexed portion of the oligonucleotide is complementary to at least 14 contiguous nucleotides within a miRNA. In some embodiments, the duplexed portion of the oligonucleotide is complementary to at least 15 contiguous nucleotides within a miRNA. In some embodiments, the duplexed portion of the oligonucleotide is complementary to at least 16 contiguous nucleotides within a miRNA. In some embodiments, the duplexed portion of the oligonucleotide is complementary to at least 17 contiguous nucleotides within a miRNA. In some embodiments, the duplexed portion of the oligonucleotide is complementary to at least 18 contiguous nucleotides within a miRNA. In some embodiments, the duplexed portion of the oligonucleotide is complementary to at least 19 contiguous nucleotides within a miRNA. In some embodiments, the duplexed portion of the oligonucleotide is complementary to at least 20 contiguous nucleotides within a miRNA. In some embodiments, the duplexed portion of the oligonucleotide is complementary to at least 21 contiguous nucleotides within a miRNA. In some embodiments, the duplexed portion of the oligonucleotide is complementary to at least 22 contiguous nucleotides within a miRNA. In some embodiments, the duplexed portion of the oligonucleotide is complementary to at least 23 contiguous nucleotides within a miRNA. In some embodiments, the duplexed portion of the oligonucleotide is complementary to at least 24 contiguous nucleotides within a miRNA. In some embodiments, the duplexed portion of the oligonucleotide is complementary to at least 25 contiguous nucleotides within a miRNA. In some embodiments, the duplexed portion of the oligonucleotide is complementary to at least 26 contiguous nucleotides within a miRNA. In some embodiments, the duplexed portion of the oligonucleotide is complementary to at least 27 contiguous nucleotides within a miRNA. In some embodiments, the duplexed portion of the oligonucleotide is complementary to at least 28 contiguous nucleotides within a miRNA. In some embodiments, the duplexed portion of the oligonucleotide is complementary to at least 29 contiguous nucleotides within a miRNA. In some embodiments, the duplexed portion of the oligonucleotide is complementary to at least 30 contiguous nucleotides within a miRNA. In some embodiments, the duplexed portion of the oligonucleotide is at least 50%, 60%, 70%, 75%, 80%, 85%, 90%, 95%, or at least 100% complementary to said miRNA. In some embodiments, the duplexed portion of the oligonucleotide is at least 50% complementary to said miRNA. In some embodiments, the duplexed portion of the oligonucleotide is at least 60% complementary to said miRNA. In some embodiments, the duplexed portion of the oligonucleotide is at least 70% complementary to said miRNA. In some embodiments, the duplexed portion of the oligonucleotide is at least 75% complementary to said miRNA. In some embodiments, the duplexed portion of the oligonucleotide is at least 80% complementary to said miRNA. In some embodiments, the duplexed portion of the oligonucleotide is at least 85% complementary to said miRNA. In some embodiments, the duplexed portion of the oligonucleotide is at least 90% complementary to said miRNA. In some embodiments, the duplexed portion of the oligonucleotide is at least 95% complementary to said miRNA. In a preferred embodiment, the duplexed portion of the oligonucleotide is at least 100% complementary to said miRNA. In some embodiments, duplexed portion of the oligonucleotide comprises between 0 and 5 mismatched base pairs. In some embodiments, duplexed portion of the oligonucleotide comprises less than 5 mismatched base pairs. In some embodiments, duplexed portion of the oligonucleotide comprises less than 4 mismatched base pairs. In some embodiments, duplexed portion of the oligonucleotide comprises less than 3 mismatched base pairs. In some embodiments, duplexed portion of the oligonucleotide comprises less than 2 mismatched base pairs. In some embodiments, duplexed portion of the oligonucleotide comprises less than 1 mismatched base pairs. In a preferred embodiment, the duplexed portion of the oligonucleotide does not comprise mismatched base pairs.

In some embodiments, the single-stranded 5′ uncapped triphosphate or biphosphate modified RNA oligonucleotide is capable of forming a duplex with a miRNA, and competes with endogenous mRNA to bind said miRNA. In some embodiments, the duplex is not cleaved by AGO2. In some embodiments, the duplex activates RIG-I. In some embodiments, the RIG-I activation is at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, or 200% greater than activation by a corresponding unmodified monophosphate RNA oligonucleotide. In some embodiments, the RIG-I activation is at least 20% greater than activation by a corresponding unmodified monophosphate RNA oligonucleotide. In some embodiments, the RIG-I activation is at least 25% greater than activation by a corresponding unmodified monophosphate RNA oligonucleotide. In some embodiments, the RIG-I activation is at least 30% greater than activation by a corresponding unmodified monophosphate RNA oligonucleotide. In some embodiments, the RIG-I activation is at least 35% greater than activation by a corresponding unmodified monophosphate RNA oligonucleotide. In some embodiments, the RIG-I activation is at least 40% greater than activation by a corresponding unmodified monophosphate RNA oligonucleotide. In some embodiments, the RIG-I activation is at least 45% greater than activation by a corresponding unmodified monophosphate RNA oligonucleotide. In some embodiments, the RIG-I activation is at least 45% greater than activation by a corresponding unmodified monophosphate RNA oligonucleotide. In some embodiments, the RIG-I activation is at least 50% greater than activation by a corresponding unmodified monophosphate RNA oligonucleotide. In some embodiments, the RIG-I activation is at least 55% greater than activation by a corresponding unmodified monophosphate RNA oligonucleotide. In some embodiments, the RIG-I activation is at least 60% greater than activation by a corresponding unmodified monophosphate RNA oligonucleotide. In some embodiments, the RIG-I activation is at least 65% greater than activation by a corresponding unmodified monophosphate RNA oligonucleotide. In some embodiments, the RIG-I activation is at least 70% greater than activation by a corresponding unmodified monophosphate RNA oligonucleotide. In some embodiments, the RIG-I activation is at least 75% greater than activation by a corresponding unmodified monophosphate RNA oligonucleotide. In some embodiments, the RIG-I activation is at least 80% greater than activation by a corresponding unmodified monophosphate RNA oligonucleotide. In some embodiments, the RIG-I activation is at least 85% greater than activation by a corresponding unmodified monophosphate RNA oligonucleotide. In some embodiments, the RIG-I activation is at least 90% greater than activation by a corresponding unmodified monophosphate RNA oligonucleotide. In some embodiments, the RIG-I activation is at least 95% greater than activation by a corresponding unmodified monophosphate RNA oligonucleotide. In some embodiments, the RIG-I activation is at least 100% greater than activation by a corresponding unmodified monophosphate RNA oligonucleotide. In some embodiments, the RIG-I activation is at least 110% greater than activation by a corresponding unmodified monophosphate RNA oligonucleotide. In some embodiments, the RIG-I activation is at least 120% greater than activation by a corresponding unmodified monophosphate RNA oligonucleotide. In some embodiments, the RIG-I activation is at least 130% greater than activation by a corresponding unmodified monophosphate RNA oligonucleotide. In some embodiments, the RIG-I activation is at least 140% greater than activation by a corresponding unmodified monophosphate RNA oligonucleotide. In some embodiments, the RIG-I activation is at least 150% greater than activation by a corresponding unmodified monophosphate RNA oligonucleotide. In some embodiments, the RIG-I activation is at least 200% greater than activation by a corresponding unmodified monophosphate RNA oligonucleotide. In preferred embodiments, the RIG-I activation elicits a tumor-specific immune response.

The oligonucleotide linked to the nanoparticle core may comprise modifications. As disclosed herein, modifications may include chemical modification, addition, deletion, substitution, or manipulation of the nucleic acid phosphate backbone, nucleic acid sugar, nucleic acid base, and/or the 5′ or 3′ end of the oligonucleotide. Oligonucleotides, especially those implemented in or as therapeutics, are generally modified on the phosphate backbone and/or ribose sugars to increase nuclease resistance and enhance affinity for target RNAs. A phosphorothioate (PS) backbone modification replaces a non-bridging oxygen atom with a sulfur atom and extends half-life of oligonucleotides in plasma from minutes to days. Enhanced protein binding has also been reported for oligonucleotides with PS-modifications compared to those with phosphodiester (PO) linkages. Further improvement of nuclease stability and binding affinity to target RNAs of oligonucleotides may be obtained by 2′ ribose modifications such as 2′-O-methyl, 2′-fluoro (2′-F), 2′-O-methoxyethyl (2′-MOE), 2′,4′-constrained 2′-O-ethyl (cEt) and locked nucleic acid (LNA). The positions of 2′ modifications within an oligonucleotide sequence can further influence protein—oligonucleotide interactions.

In certain embodiments, the single-stranded 5′ uncapped triphosphate or biphosphate modified RNA oligonucleotides is complementary to an endogenous tumor-specific RNA which is highly expressed in tumor cells in comparison to non-tumor cells. In certain embodiments, the single-stranded 5′ uncapped triphosphate or biphosphate modified RNA oligonucleotide comprises a sequence which is complementary to an endogenous tumor-specific RNA. In some embodiments, the single-stranded 5′ uncapped triphosphate or biphosphate modified RNA oligonucleotide comprises other modifications.

In some embodiments, the single-stranded 5′ uncapped triphosphate or biphosphate modified RNA oligonucleotide further comprises a 2′-fluoro (2′-F) ribose modification. In some embodiments, the 2′-F ribose modification is present when the corresponding base is a cytosine or a uracil. In some embodiments, the 2′-F ribose modification is present at the 10^{th} or 11^{th} nucleotide from the 5′-terminus of the modified RNA oligonucleotide. In some embodiments, the single-stranded 5′ uncapped triphosphate or biphosphate modified RNA oligonucleotide further comprises a phosphorothioate (PS) backbone modification. In some embodiments, the single-stranded 5′ uncapped triphosphate or biphosphate modified RNA oligonucleotide further comprises a 2′-fluoro (2′-F) ribose modification and a phosphorothioate (PS) backbone modification.

In a preferred embodiment, the single-stranded 5′ uncapped triphosphate or biphosphate modified RNA oligonucleotide does not comprise any other modifications. In a preferred embodiment, the single-stranded 5′ uncapped triphosphate or biphosphate modified RNA oligonucleotide does not comprise any modifications selected from the group consisting of: 2′-O-methyl (2′-OMe) ribose modification, N-6-methyladenosine (m6A), pseudouridine (Ψ), N-1-methylpseudouridine (mΨ), N-1-methylpseudouridine (mΨ), 5-methyl-cytidine (5mC), 5-hydroxymethyl-cytidine (5hmC), or 5-methoxycytidine (5moC). In some embodiments the single-stranded 5′ uncapped triphosphate or biphosphate modified RNA oligonucleotide does not comprise 2′-O-methyl (2′-OMe) ribose modification. In some embodiments, the single-stranded 5′ uncapped triphosphate or biphosphate modified RNA oligonucleotide does not comprise a N-6-methyladenosine (m6A). In some embodiments, the single-stranded 5′ uncapped triphosphate or biphosphate modified RNA oligonucleotide does not comprise a pseudouridine (Ψ). In some embodiments, the single-stranded 5′ uncapped triphosphate or biphosphate modified RNA oligonucleotide does not comprise a N-1-methylpseudouridine (mΨ). In some embodiments, the single-stranded 5′ uncapped triphosphate or biphosphate modified RNA oligonucleotide does not comprise a 5-methyl-cytidine (5mC). In some embodiments, the single-stranded 5′ uncapped triphosphate or biphosphate modified RNA oligonucleotide does not comprise a 5-hydroxymethyl-cytidine (5hmC). In some embodiments, the single-stranded 5′ uncapped triphosphate or biphosphate modified RNA oligonucleotide does not comprise a 5-methoxycytidine (5moC).

In some embodiments, the single-stranded 5′ uncapped triphosphate or biphosphate modified RNA oligonucleotide comprises one or more modifications. In some embodiments, the single-stranded 5′ uncapped triphosphate or biphosphate modified RNA oligonucleotide comprises one or more modifications selected from the group consisting of: phosphorothioate (PS) backbone modification, 2′-O-methyl (2′-OMe) ribose modification, N-6-methyladenosine (m6A), pseudouridine (Ψ), N-1-methylpseudouridine (mΨ), N-1-methylpseudouridine (mΨ), 5-methyl-cytidine (5mC), 5-hydroxymethyl-cytidine (5hmC), or (5moC). In some embodiments the single-stranded 5′ uncapped triphosphate or biphosphate modified RNA oligonucleotide comprises 2′-O-methyl (2′-OMe) ribose modification. In some embodiments, the single-stranded 5′ uncapped triphosphate or biphosphate modified RNA oligonucleotide comprises a N-6-methyladenosine (m6A). In some embodiments, the single-stranded 5′ uncapped triphosphate or biphosphate modified RNA oligonucleotide comprises a pseudouridine (Ψ). In some embodiments, the single-stranded 5′ uncapped triphosphate or biphosphate modified RNA oligonucleotide comprises a N-1-methylpseudouridine (mΨ). In some embodiments, the single-stranded 5′ uncapped triphosphate or biphosphate modified RNA oligonucleotide comprises a 5-methyl-cytidine (5mC). In some embodiments, the single-stranded 5′ uncapped triphosphate or biphosphate modified RNA oligonucleotide comprises a 5-hydroxymethyl-cytidine (5hmC). In some embodiments, the single-stranded 5′ uncapped triphosphate or biphosphate modified RNA oligonucleotide comprises 5-methoxycytidine (5moC).

In some embodiments, the single-stranded 5′ uncapped triphosphate or biphosphate modified RNA oligonucleotide comprises a sequence which is at least 10 nucleotides in length. In some embodiments, the oligonucleotide comprises a sequence which is at least 15 nucleotides in length. In some embodiments, the oligonucleotide comprises a sequence which is at least 16 nucleotides in length. In some embodiments, the oligonucleotide comprises a sequence which is at least 17 nucleotides in length. In some embodiments, the oligonucleotide comprises a sequence which is at least 18 nucleotides in length. In some embodiments, the oligonucleotide comprises a sequence which is at least 19 nucleotides in length. In some embodiments, the oligonucleotide comprises a sequence which is at least 20 nucleotides in length. In some embodiments, the oligonucleotide comprises a sequence which is at least 21 nucleotides in length. In some embodiments, the oligonucleotide comprises a sequence which is at least 22 nucleotides in length. In some embodiments, the oligonucleotide comprises a sequence which is at least 23 nucleotides in length. In some embodiments, the oligonucleotide comprises a sequence which is at least 24 nucleotides in length. In some embodiments, the oligonucleotide comprises a sequence which is at least 25 nucleotides in length. In some embodiments, the oligonucleotide comprises a sequence which is at least 26 nucleotides in length. In some embodiments, the oligonucleotide comprises a sequence which is at least 27 nucleotides in length. In some embodiments, the oligonucleotide comprises a sequence which is at least 28 nucleotides in length. In some embodiments, the oligonucleotide comprises a sequence which is at least 29 nucleotides in length. In some embodiments, the oligonucleotide comprises a sequence which is at least 30 nucleotides in length. In some embodiments, the oligonucleotide comprises a sequence which is at least 50 nucleotides in length. In some embodiments, the oligonucleotide comprises a sequence which is between 15 and 50 nucleotides in length. In some embodiments, the oligonucleotide comprises a sequence which is between 15 and 30 nucleotides in length. In some embodiments, the oligonucleotide comprises a sequence which is between 15 and 29 nucleotides in length. In some embodiments, the oligonucleotide comprises a sequence which is between 15 and 28 nucleotides in length. In some embodiments, the oligonucleotide comprises a sequence which is between 15 and 27 nucleotides in length. In some embodiments, the oligonucleotide comprises a sequence which is between 15 and 26 nucleotides in length. In some embodiments, the oligonucleotide comprises a sequence which is between 15 and 25 nucleotides in length. In some embodiments, the oligonucleotide comprises a sequence which is between 16 and 50 nucleotides in length. In some embodiments, the oligonucleotide comprises a sequence which is between 16 and 30 nucleotides in length. In some embodiments, the oligonucleotide comprises a sequence which is between 16 and 29 nucleotides in length. In some embodiments, the oligonucleotide comprises a sequence which is between 16 and 28 nucleotides in length. In some embodiments, the oligonucleotide comprises a sequence which is between 16 and 27 nucleotides in length. In some embodiments, the oligonucleotide comprises a sequence which is between 16 and 26 nucleotides in length. In some embodiments, the oligonucleotide comprises a sequence which is between 16 and 25 nucleotides in length.

The 5′pp and 5′ppp anti-miRNAs/mRNAs comprise sequences that are complementary to at least 10 (e.g., at least 11, 12, 13, 14, 15, 16, 17, 18, 19, 20, 21, 22, or 23) contiguous nucleotides within a miRNA or mRNA. Exemplary miRNAs include, e.g., miR-9; miR-10b; miR-21; miR-106b/93; miR-125b; miR-130a; miR-155; miR-181a; miR-200s; miR-210-3p; miR-221/222; miR-335; miR-498; miR-504; miR-1810; miR-1908; miR-224/452; or miR-181/340 (see, e.g., Table 1 of Nguyen and Chang, Int J Mol Sci. 2017; 19(1):65) and those listed in Table 1 and Table 2 herein. Exemplary mRNAs include those listed in Table 2 herein.

In certain embodiments, the nanoparticles comprise a single-stranded 5′ uncapped triphosphate modified RNA oligonucleotide. In certain embodiments, the nanoparticles comprise a single-stranded 5′ uncapped biphosphate modified RNA oligonucleotide. In some embodiments, the single-stranded 5′ uncapped triphosphate or biphosphate modified RNA oligonucleotide comprises a nucleic acid sequence that is at least 75% identical to a nucleic acid sequence selected from SEQ ID NOs: 1, 2, 3, 4, 5, 6, 7, 8, 9, 11, 12, or 13. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 80% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 85% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 90% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 95% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 97% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 98% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 99% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is 100% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide consists of a nucleic acid sequence that is identical to a nucleic acid sequence selected from SEQ ID NOs: 1, 2, 3, 4, 5, 6, 7, 8, 9, 10, 11, 12, or 13.

In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 1. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 2. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 3. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 4. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 5. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 6. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 7. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 8. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 9. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 10. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 11. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 12. In some embodiments, the single-stranded 5′ uncapped triphosphate or biphosphate modified RNA oligonucleotide comprises a nucleic acid sequence that is at least 75%, 80%, 85%, 90%, 95%, 97%, 98%, 99% or 100% identical to the nucleic acid sequence of SEQ ID NO: 13.

In some embodiments, the single-stranded 5′ uncapped triphosphate or biphosphate modified RNA oligonucleotide (“modified RNA oligonucleotide”) is attached to the nanoparticle core. In certain embodiments, the nanoparticle comprises up to 40 different modified RNA oligonucleotides. In certain embodiments, the nanoparticle comprises up to different modified RNA oligonucleotides. In certain embodiments, the nanoparticle comprises up to 30 different modified RNA oligonucleotides. In certain embodiments, the nanoparticle comprises up to 25 different modified RNA oligonucleotides. In certain embodiments, the nanoparticle comprises up to 20 different modified RNA oligonucleotides. In certain embodiments, the nanoparticle comprises up to 15 different modified RNA oligonucleotides. In certain embodiments, the nanoparticle comprises up to 10 different modified RNA oligonucleotides. In certain embodiments, the nanoparticle comprises up to 9 different modified RNA oligonucleotides. In certain embodiments, the nanoparticle comprises up to 8 different modified RNA oligonucleotides. In certain embodiments, the nanoparticle comprises up to 7 different modified RNA oligonucleotides. In certain embodiments, the nanoparticle comprises up to 6 different modified RNA oligonucleotides. In certain embodiments, the nanoparticle comprises up to 5 different modified RNA oligonucleotides. In certain embodiments, the nanoparticle comprises up to 4 different modified RNA oligonucleotides. In certain embodiments, the nanoparticle comprises up to 3 different modified RNA oligonucleotides. In certain embodiments, the nanoparticle comprises up to 2 different modified RNA oligonucleotides. In certain embodiments, the nanoparticle comprises 1 modified RNA oligonucleotide.

In some embodiments, the single-stranded 5′ uncapped triphosphate or biphosphate modified RNA oligonucleotide is attached to the nanoparticle core (e.g., to the polymer coating of the nanoparticle core) through a chemical moiety that contains a thioether bond or a disulfide bond, in some embodiments, the single-stranded 5′ uncapped triphosphate or biphosphate modified RNA oligonucleotide is attached to the nanoparticle core through a chemical moiety that contains an amide bond. Additional chemical moieties that can be used to covalently link a nucleic acid (e.g., the single-stranded 5′ uncapped triphosphate or biphosphate modified RNA oligonucleotide) to a nanoparticle core are known in the art.

A variety of different methods can be used to covalently link a nucleic acid (e.g., the single-stranded 5′ uncapped triphosphate or biphosphate modified RNA oligonucleotide) to a nanoparticle core. Non-limiting examples of methods that can be used to link a nucleic acid to a nanoparticle core are described in EP 0937097; U.S. RE41005; Lund et al., Nucleic Acid Res. 16: 10861, 1998; Todt et al., Methods Mol. Biol. 529:81-100, 2009; Brody et al., J. Biotechnol. 74:5-13, 2000; Ghosh et al., Nucleic Acids Res. 15:5353-5372, 1987; U.S. Pat. Nos. 5,900,481; 7,569,341; 6,995,248; 6,818,394; 6,811,980; 5,900,481; and 4,818,681 (each of which is incorporated by reference in its entirety). In some embodiments, carbodiimide is used for the end-attachment of a nucleic acid to a nanoparticle core. In some embodiments, the nucleic acid is attached to the nanoparticle core through the reaction of one of its bases with an activated moiety present on the surface of the nanoparticle core (e.g., the reaction of an electrophilic base with a nucleophilic moiety on the surface of the nanoparticle core, or the reaction of a nucleophilic base with an electrophilic residue on the surface of the nanoparticle core). In some embodiments, a 5′-NEE modified nucleic acid is attached to a nanoparticle core containing CNBr-activated hydroxyl groups (see, e.g., Lund et al., supra). Additional methods for attaching an amino-modified nucleic acid to a nanoparticle core are described below. In some embodiments, a 5′-phosphate nucleic acid is attached to a nanoparticle core containing hydroxyl groups in the presence of a carbodiimide (see, e.g., Lund et al., supra). Other methods of attaching a nucleic acid to a nanoparticle core include carbodiimide-mediated attachment of a 5′-phosphate nucleic acid to a NEE group on a nanoparticle core, and carbodiimide-mediated attachment of a 5′-NEE nucleic acid to a nanoparticle core having carboxyl groups (see, e.g., Lund et al., supra).

In exemplary methods, a nucleic acid can be produced that contains a reactive amine or a reactive thiol group. The amine or thiol in the nucleic acid can be linked to another reactive group. The two common strategies to perform this reaction are to link the nucleic acid to a similar reactive moiety (amine to amine or thiol to thiol), which is called homobifunctional linkage, or to link to the nucleic acid to an opposite group (amine to thiol or thiol to amine), known as heterobifunctional linkage. Both techniques can be used to attach a nucleic acid to a nanoparticle core (see, for example, Misra et al., Bioorg. Med. Chem. Lett. 18:5217-5221, 2008; Mirsa et al., Anal. Biochem. 369:248-255, 2007; Mirsa et al., Bioorg. Med. Chem. Lett. 17:3749-3753, 2007; and Choithani et al., Methods Mol Biol. 381:133-163, 2007).

Traditional attachment techniques, especially for amine groups, have relied upon homobifunctional linkages. One of the most common techniques has been the use of bisaldehydes such as glutaraldehyde. Disuccinimidyl suberate (DSS), commercialized by Syngene (Frederick, MD) as synthetic nucleic acid probe (SNAP) technology, or the reagent p-phenylene diisothiocyanate can also be used to generate a covalent linkage between the nucleic acid and the nanoparticle core. N,N′-o-phenylenedimaleimide can be used to cross-link thiol groups. With all of the homobifunctional cross-linking agents, the nucleic acid is initially activated and then added to the nanoparticle core (see, for example, Swami et al., Int. J. Pharm. 374: 125-138, 2009, Todt et al., Methods Mol. Biol. 529:81-100, 2009; and Limanskii, Biofizika 51:225-235, 2006).

Heterobifunctional linkers can also be used to attach a nucleic acid to a nanoparticle core. For example, N-succinimidyl-3-(2-pyridyldithio)proprionate (SPDP) initially links to a primary amine to give a dithiol-modified compound. This can then react with a thiol to exchange the pyridylthiol with the incoming thiol (see, for example, Nostrum et al., J. Control Release 15; 153(1): 93-102, 2011, and Berthold et al., Bioconjug. Chem. 21: 1933-1938, 2010).

An alternative approach for thiol use has been a thiol-exchange reaction. If a thiolated nucleic acid is introduced onto a disulfide nanoparticle core, a disulfide-exchange reaction can occur that leads to the nucleic acid being covalently bonded to the nanoparticle core by a disulfide bond. A multitude of potential cross-linking chemistries are available for the heterobifunctional cross-linking of amines and thiols.

Generally, these procedures have been used with a thiolated nucleotide. Reagents typically employed have been NHS (N-hydroxysuccinimide ester), MBS (m-maleimidobenzoyl-N-succinimide ester), and SPDP (a pyridyldisulfide-based system). The heterobifunctional linkers commonly used rely upon an aminated nucleic acid.

Additional methods for covalently linking a nucleic acid to a nanoparticle core are known in the art.

Nanoparticle Core

In some embodiments, the nanoparticles provided herein comprise a nanoparticle core. In some embodiments, the nanoparticle or nanoparticle core has a diameter of between about 2 nm to about 200 nm (e.g., between about 10 nm to about 30 nm, between about 5 nm to about 25 nm, between about 10 nm to about 25 nm, between about 15 nm to about 25 nm, between about 20 nm and about 25 nm, between about 25 nm to about 50 nm, between about 50 nm and about 200 nm, between about 70 nm and about 200 nm, between about 80 nm and about 200 nm, between about 100 nm and about 200 nm, between about 140 nm to about 200 nm, and between about 150 nm to about 200 nm), and contain a polymer coating.

In some embodiments, the nanoparticle has a diameter that is about 18% to about 28% (e.g., about 18% to about 23%, about 20% to about 23%, about 23% to about 25%, about 23% to about 28%) greater than a diameter of a nanoparticle that does not include a radiolabel. In some embodiments, the nanoparticles can have a diameter that is about 23% greater than a diameter of a nanoparticle that does not include a radiolabel. In some embodiments, the nanoparticles can accumulate in a metastatic tissue of a subject at a similar rate than metastatic-targeting nanoparticles that do not include a radiolabel. In some embodiments, the nanoparticles exhibit about the same accumulation in a metastatic tissue of a subject as compared to metastatic-targeting nanoparticles that do not include a radiolabel.

In some embodiments, the nanoparticle or nanoparticle core is spherical or ellipsoidal, or has an amorphous shape. In some embodiments, the nanoparticle or nanoparticle core has a diameter (between any two points on the exterior surface of the nanoparticle) of between about 2 nm to about 200 nm (e.g., between about 10 nm to about 200 nm, between about 2 nm to about 30 nm, between about 5 nm to about 25 nm, between about 10 nm to about 25 nm, between about 15 nm to about 25 nm, between about 20 nm to about 25 nm, between about 50 nm to about 200 nm, between about 70 nm to about 200 nm, between about 80 nm to about 200 nm, between about 100 nm to about 200 nm, between about 140 nm to about 200 nm, and between about 150 nm to about 200 nm). In some embodiments, a nanoparticle or nanoparticle core with a diameter of between about 2 nm to about 30 nm can localize to the lymph nodes in a subject. In some embodiments, a nanoparticle or nanoparticle core with a diameter of between about 40 nm to about 200 nm can localize to the liver.

In some embodiments, a nanoparticle or nanoparticle core contains, in part, a core of containing a polymer (e.g., poly(lactic-co-glycolic acid)). Skilled practitioners will appreciate that any number of art known materials can be used to prepare nanoparticle cores, including, but are not limited to, gums (e.g., Acacia, Guar), chitosan, gelatin, sodium alginate, and albumin. Additional polymers that can be used to generate the nanoparticle cores described herein are known in the art. For example, polymers that can be used to generate the nanoparticle cores include, but are not limited to, cellulosics, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate and polycaprolactone.

Skilled practitioners will appreciate that the material used in the composition of the nanoparticle core, the methods for preparing, coating, and methods for controlling the size of the nanoparticle core can vary substantially. However, these methods are well known to those in the art. Key issues include the biodegradability, toxicity profile, and pharmacokinetics/pharmacodynamics of the nanoparticles. The composition and/or size of the nanoparticle core are key determinants of their biological fate. For example, a larger nanoparticle core is typically taken up and degraded by the liver, whereas a smaller nanoparticle core (<30 nm in diameter) will typically circulate for a long time (sometimes over 24-hr blood half-life in humans) and accumulate in lymph nodes and the interstitium of organs with hyperpermeable vasculature, such as tumors.

In some embodiments, the nanoparticle core is magnetic (e.g., contains a core of a magnetic material). In some embodiments, a nanoparticle described herein has a core of magnetic material (e.g., a magnetic nanoparticle). In some embodiments, the magnetic nanoparticle core includes ferric chloride, ferrous chloride, or a combination thereof, and a dextran coating. In some embodiments, the magnetic nanoparticle core contains a mixture of two or more of the different nanoparticle cores described herein. In some embodiments, the mixture of two or more different nanoparticle cores contain at least one magnetic nanoparticle core having a tunable surface functionalization, and at least one magnetic nanoparticle core having tunable magnetic properties.

In some embodiments, any of the nanoparticles described herein can contain a core of a magnetic material (e.g., a therapeutic magnetic nanoparticle). In some embodiments, the magnetic material or particle can contain a diamagnetic, paramagnetic, superparamagnetic, or ferromagnetic material that is responsive to a magnetic field. In some embodiments, a magnetic nanoparticle core comprises a metal oxide. In some embodiments, the metal oxide is selected from the group consisting of: magnetite; ferrites (e.g., ferrites of manganese, cobalt, and nickel); Fe(II) oxides, and hematite, and metal alloys thereof. The core of magnetic material can be formed by converting metal salts to metal oxides using methods known in the art (e.g., Kieslich et al., Inorg. Chem. 2011). In some embodiments, the nanoparticles contain cyclodextrin gold or quantum dots. Non-limiting examples of methods that can be used to generate magnetic nanoparticle cores are described in Medarova et al., Methods Mol. Biol. 555:1-13, 2009; and Medarova et al., Nature Protocols 1:429-431, 2006.

Additional magnetic materials and methods of making magnetic materials are known in the art. In some embodiments, the position or localization of a magnetic nanoparticle can be imaged in a subject (e.g., imaged in a subject following the administration of one or more doses of a magnetic nanoparticle).

In some embodiments, the magnetic nanoparticle core is functionalized with one or more amine groups. In some embodiments, the functionalization occurs at the surface of the magnetic nanoparticle core. In some embodiments, the one or more amine groups are covalently linked to the dextran coating. In some embodiments, the one or more amine groups substitute one or more hydroxyl groups of the dextran coating. In some embodiments, the number of the one or more amine groups is tunable based on a concentration of ferric chloride, ferrous chloride, or a combination thereof. In some embodiments, the nanoparticle composition includes about 5 to about 1000 amine groups. In some embodiments, the nanoparticle core includes about 5 to 25, 25 to 100, 100 to 150, 150 to 200, 200 to 250, 250 to 300, 300 to 350, 350 to 400, 450 to 500, 500 to 550, 550 to 600, 600 to 650, 650 to 700, 700 to 750, 750 to 800, 800 to 850, 850 to 900, 900 to 950, or 950 to 1000 amine groups.

In some embodiments, the magnetic nanoparticle core comprises a core of a magnetic material (e.g., ferric chloride and/or ferrous chloride). In some embodiments, the magnetic nanoparticle core comprises about 0.60 g to about 0.70 g of ferric chloride and about 0.3 g to about 0.5 g of ferrous chloride. In some embodiments, the magnetic nanoparticle core comprising about 0.60 g to about 0.70 g of ferric chloride and about 0.3 g to about 0.5 g of ferrous chloride is functionalized with about 5 to 150 amine groups. In some embodiments, the magnetic nanoparticle core comprises about 0.65 g of ferric chloride and about 0.4 g of ferrous chloride. In some embodiments, the magnetic nanoparticle core comprising about 0.65 g of ferric chloride and about 0.4 g of ferrous chloride is functionalized with about 60 to 90 amine groups. In some embodiments, the magnetic nanoparticle core comprising about 0.65 g of ferric chloride and about 0.4 g of ferrous chloride is functionalized with about 5 to 150 amine groups. In some embodiments, the magnetic nanoparticle core comprising about 0.65 g of ferric chloride and about 0.4 g of ferrous chloride is functionalized with about 1 to 150 amine groups. In some embodiments, the magnetic nanoparticle core comprising about 0.65 g of ferric chloride and about 0.4 g of ferrous chloride is functionalized with about 1 to 10 amine groups, about 10 to 20 amine groups, about 20 to 30 amine groups, about 30 to 40 amine groups, about 40 to 50 amine groups, about 50 to 60 amine groups, about 60 to 70 amine groups, about 70 to 80 amine groups, about 80 to 90 amine groups, about 90 to 100 amine groups, about 100 to 110 amine groups, about 110 to 120 amine groups, about 120 to 130 amine groups, about 130 to 140 amine groups, or about 140 to 150 amine groups.

In some embodiments, the magnetic nanoparticle core comprises about 1 g to about 1.4 g of ferric chloride. In some embodiments, the magnetic nanoparticle core comprising about 1 g to about 1.4 g of ferric chloride is functionalized with about 246 to 500 amine groups. In some embodiments, the magnetic nanoparticle core comprises about 1.2 g of ferric chloride. In some embodiments, the magnetic nanoparticle core comprising about 1.2 g of ferric chloride is functionalized with about 246 to 500 amine groups. In some embodiments, the magnetic nanoparticle core functionalized with about 246 to 500 amine groups does not comprise ferric chloride. In some embodiments, the magnetic nanoparticle core comprising about 1.2 g of ferric chloride is functionalized with about 200 to 600 amine groups. In some embodiments, the magnetic nanoparticle core comprising about 1.2 g of ferric chloride is functionalized with about 200 to 250 amine groups, about 250 to 300 amine groups, about 300 to 350 amine groups, about 350 to 400 amine groups, about 400 to 450 amine groups, about 450 to 500 amine groups, about 500 to 550 amine groups, about 550 to 600 amine groups, or more.

Thus, in some embodiments, the number of amine groups conjugated to the dextran coating can be fine-tuned by controlling the concentrations of ferric chloride and ferrous chloride, which are used to prepare the magnetic nanoparticle core.

In some embodiments, the magnetic nanoparticle core has a magnetic strength that is tunable based on a concentration of ferric chloride, ferrous chloride, or a combination thereof.

In some embodiments, the magnetic nanoparticle core comprises about 0.1% to about 99.9% of ferric ion and about 99.9% to about 0.1% of ferrous ion in total iron per MNP. In some embodiments, the magnetic nanoparticle core comprises about 60% to about 80% of ferric chloride and about 20% to about 40% of ferrous chloride have stronger magnetic properties than nanoparticle cores having a ferrous chloride amount higher than about 80%. In some embodiments, the magnetic nanoparticle core comprises about 70% ferric ion and about 30% g ferrous ion have stronger magnetic properties than magnetic nanoparticle cores having a ferrous ion amount higher than about 30%.

In some embodiments, the magnetic nanoparticle core has a non-linearity index (NLI) ranging from about 6 to about 40. In some embodiments, the magnetic nanoparticle core has an NLI ranging from about 6 to about 70. In some embodiments, the magnetic nanoparticle core has an NLI ranging from about 8.5 to about 14.8. In some embodiments, the magnetic nanoparticle core has an NLI ranging from about 8 to about 14. In some embodiments, the magnetic nanoparticle core has an NLI of about 6. In some embodiments, the magnetic nanoparticle core has an NLI of about 8. In some embodiments, the magnetic nanoparticle core has an NLI of about 14. In some embodiments, the magnetic nanoparticle core has an NLI of about 67. In some embodiments, the magnetic nanoparticle core has an NLI ranging from 6 to 7; 7 to 8; 8 to 9; 9 to 10; 10 to 11; 11 to 12; 12 to 13; 13 to 14; 14 to 15; 15 to 16; 16 to 17; 17 to 18; 18 to 19; 19 to 20; 20 to 30; 30 to 40; 40 to 50; 50 to 60; or 60 to 70. In some embodiments, the magnetic nanoparticle core comprises about g of ferric chloride and about 0.2 g of ferrous chloride. In some embodiments, the magnetic nanoparticle core comprising about 0.54 g of ferric chloride and about 0.2 g of ferrous chloride has a NLI ranging from about 8.5 to about 14.8. In some embodiments, the magnetic nanoparticle core comprising about 0.54 g of ferric chloride and about 0.2 g of ferrous chloride has a NLI of about 12. In some embodiments, the magnetic strength of the magnetic nanoparticle core can be quantified by measuring a non-linearity index (NLI) by magnetic particle spectrometry as described in WO 2021/113829.

In some embodiments, the magnetic nanoparticle core comprises about 80% to about 100% of ferric chloride and about 20% to about 0% of ferrous chloride. In some embodiments, the magnetic nanoparticle core comprises about 0% to about 50% of ferric chloride and about 100% to about 50% of ferrous chloride. In some embodiments, the magnetic nanoparticle core comprising about 0% to about 50% of ferric chloride and about 100% to about 50% of ferrous chloride has weaker magnetic properties than magnetic nanoparticle cores having a ferrous chloride amount lower than about 0.4 g. In some embodiments, the magnetic nanoparticle cores comprising about 0.54 g of ferric chloride and about 0.4 g of ferrous chloride have weaker magnetic properties than magnetic nanoparticle cores having a ferrous chloride amount lower than about 0.2 g. In some embodiments, the magnetic nanoparticle cores comprising about 0.54 g of ferric chloride and about 0.4 g of ferrous chloride has a NLI ranging from about 50 to about 120. In some embodiments, the magnetic nanoparticle cores comprising about 0.54 g of ferric chloride and about 0.4 g of ferrous chloride has a NLI of about 67.

Thus, in some embodiments, the magnetic properties (e.g., magnetic strength) of the magnetic nanoparticle cores can be fine-tuned by controlling the concentrations of ferric chloride and ferrous chloride, which are used to prepare the magnetic nanoparticle core.

In some embodiments, the magnetic nanoparticle core has an iron concentration ranging from about 8 mM to about 217 mM. In some embodiments, the magnetic nanoparticle core has an iron concentration ranging from about 8 mM to about 15 mM, about 15 mM to about 25 mM, about 25 mM to about 50 mM, about 50 mM to about 60 mM, about 60 mM to about 70 mM, about 70 mM to about 80 mM, about 80 mM to about 90 mM, about 90 mM to about 100 mM, about 100 mM to about 110 mM, about 110 mM to about 120 mM, about 120 mM to about 130 mM, about 130 mM to about 140 mM, about 140 mM to about 150 mM, about 150 mM to about 160 mM, about 160 mM to about 170 mM, about 170 mM to about 180 mM, about 180 mM to about 190 mM, about 190 mM to about 200 mM, about 200 mM to about 210 mM, and about 210 mM to about 220 mM.

In some embodiments, the magnetic nanoparticle core has an iron concentration ranging from about 1 mg/mL to about 25 mg/mL. In some embodiments, the magnetic nanoparticle core has an iron concentration ranging from about 1 mg/mL to about 5 mg/mL, about 5 mg/mL to about 10 mg/mL, about 10 mg/mL to about 15 mg/mL, about 15 mg/mL to about 20 mg/mL, or about 20 mg/mL to about 25 mg/mL. In some embodiments, the nanoparticles described herein do not contain a magnetic material.

In some embodiments, the magnetic nanoparticle core is used to deliver a composition containing at least one (e.g., one, two, three, or four) of any of the single-stranded 5′ uncapped triphosphate or biphosphate modified RNA oligonucleotide (e.g., modified RNA oligonucleotide as used herein) described herein. By “at least one”, it is meant that one or more modified RNA oligonucleotide(s) of the same or different oligonucleotide(s) can be used together. In some embodiments, the magnetic nanoparticle delivers one modified RNA oligonucleotides. In some embodiments, the magnetic nanoparticle delivers two modified RNA oligonucleotides. In some embodiments, the magnetic nanoparticle delivers three modified RNA oligonucleotides. In some embodiments, the magnetic nanoparticle delivers four modified RNA oligonucleotides. In some embodiments, the magnetic nanoparticle delivers five modified RNA oligonucleotides.

Polymer Coatings of Nanoparticles

In some embodiments, the nanoparticles described herein comprise a polymer coating over the nanoparticle core (e.g., over the surface of a nanoparticle core). The polymer material can be suitable for attaching or coupling one or more biological agents (e.g., such as any of the modified oligonucleotides or radiolabels described herein). One of more biological agents (e.g., a modified oligonucleotide or radiolabel) can be fixed to the polymer coating by chemical coupling (covalent bonds).

In some embodiments, the nanoparticle core is formed by a method that includes coating the core of the nanoparticle (e.g., a magnetic material) with a polymer that is relatively stable in water. In some embodiments, the nanoparticle core is formed by a method that includes coating a material (e.g., a magnetic material) with a polymer or absorbing the material into a thermoplastic polymer resin having reducing groups thereon. A coating can also be applied to a material using the methods described in U.S. Pat. Nos. 5,834,121, 5,356,713, 5,318,797, 5,283,079, 5,232,789, 5,091,206, 4,965,007, 4,774,265, 4,770,183, 4,654,267, 4,554,088, 4,490,436, 4,336,173, and 4,421,660; and WO 10/111066 (each disclosure of which is incorporated herein by reference).

Methods for the synthesis of iron oxide nanoparticle cores include, for example, physical and chemical methods. For example, iron oxides can be prepared by co-precipitation of Fe2+ and Fe3+ salts in an aqueous solution. The resulting core consists of magnetite (Fe3O4), maghemite (γ-Fe2O3) or a mixture of the two. The anionic salt content (chlorides, nitrates, sulphates etc.), the Fe2+ and Fe3+ ratio, pH and the ionic strength in the aqueous solution all play a role in controlling the size. It is important to prevent the oxidation of the synthesized nanoparticle cores and to protect their magnetic properties by carrying out the reaction in an oxygen free environment under inert gas such as nitrogen or argon. The coating materials can be added during the co-precipitation process in order to prevent the agglomeration of the iron oxide nanoparticles into microparticles. Skilled practitioners will appreciated that any number of art known surface coating materials can be used for stabilizing iron oxide nanoparticles, among which are synthetic and natural polymers, such as, for example, polyethylene glycol (PEG), dextran, polyvinylpyrrolidone (PVP), fatty acids, polypeptides, chitosan, and/or gelatin.

For example, U.S. Pat. No. 4,421,660 (“the '660 patent”) describes that polymer coated particles of an inorganic material can be conventionally prepared by (1) treating the inorganic solid with acid, a combination of acid and base, alcohol or a polymer solution; (2) dispersing an addition polymerizable monomer in an aqueous dispersion of a treated inorganic solid; and (3) subjecting the resulting dispersion to emulsion polymerization conditions. See, e.g., col. 1, lines 21-27 of the '660 patent. The '660 patent also discloses a method for coating an inorganic nanoparticles with a polymer, which comprises the steps of (1) emulsifying a hydrophobic, emulsion polymerizable monomer in an aqueous colloidal dispersion of discrete particles of an inorganic solid; and (2) subjecting the resulting emulsion to emulsion polymerization conditions to form a stable, fluid aqueous colloidal dispersion of the inorganic solid particles dispersed in a matrix of a water-insoluble polymer of the hydrophobic monomer. See, e.g., col. 1, lines 42-50 of the '660 patent.

Alternatively, polymer-coated magnetic material can be commercially obtained that meets the starting requirements of size. For example, commercially available ultra-small superparamagnetic iron oxide nanoparticles include NC100150 Injection (Nycomed Amersham, Amersham Health) and Ferumoxytol (AMAG Pharmaceuticals, Inc.).

Suitable polymers that can be used to coat the core of material (e.g., magnetic material) include without limitation: polystyrenes, polyacrylamides, polyetherurethanes, polysulfones, fluorinated or chlorinated polymers such as polyvinyl chloride, polyethylenes, and polypropylenes, polycarbonates, and polyesters. Additional examples of polymers that can be used to coat the core of material (e.g., magnetic material) include polyolefins, such as polybutadiene, polydichlorobutadiene, polyisoprene, polychloroprene, polyvinylidene halides, polyvinylidene carbonate, and polyfluorinated ethylenes. A number of copolymers, including styrene/butadiene, alpha-methyl styrene/dimethyl siloxane, or other polysiloxanes can also be used to coat the core of material (e.g., polydimethyl siloxane, polyphenylmethyl siloxane, and polytrifluoropropylmethyl siloxane). Additional polymers that can be used to coat the core of material include polyacrylonitriles or acrylonitrile-containing polymers, such as poly alpha-acrylanitrile copolymers, alkyd or terpenoid resins, and polyalkylene polysulfonates. In some embodiments, the polymer coating is dextran.

Chelators

In some embodiments, the nanoparticles described herein comprise at least one chelator covalently-linked to the nanoparticle core. In some embodiments, the chelator forms a stable complex with a radiolabel. In some embodiments, the chelator binds to a radiolabel.

In some embodiments, the nanoparticles comprise a mixture of two or more of the different chelators described herein. In some embodiments, the nanoparticle comprises about 1 chelator to about 15 chelators e.g., about 1 chelator to about 11 chelators, about 1 chelator to about 12 chelators, about 1 chelator to about 13 chelators, about 1 chelator to about 14 chelators, about 1 chelator to about 15 chelators, about 11 chelators to about 12 chelators, about 11 chelators to about 13 chelators, about 11 chelators to about 14 chelators, or about 11 chelators to about 15 chelators) covalently-linked to each nanoparticle core. In some embodiments, the nanoparticles can include about 13 chelators covalently-linked to each nanoparticle core.

A variety of different chelators that can be covalently linked to the nanoparticle cores are known in the art. Non-limiting examples of such chelators include 1,47-triazacyclononane,1-glutaric acid-4,7-acetic acid (NODAGA), 1,4,7,10-tetraazacyclododecane-1,4,7,10-tetrayl)tetraacetic acid (DOTA), 10-(2,6-dioxotetrahydro-2H-pyran-3-yl)-1,4,7,10-tetrauzacyclododecane-1,4,7-triyptriacetic acid (DOTA-GA), 2-[4,7,10-tris(carboxymethyl)-6-[(4-isothiocyanatophenyi)methyl]-1,4,7,10-tetrazacyclododec-1-yl]acetic acid (p-SCN-Bn)-DOTA, 2,2′41,4,8,11-tetraazabicyclo[6.6.2]hexadecane-4,11-diyl)diacetic acid (CB-TE2A). CB-TE1A1P, 1,4-bis(carboxymethyl)-6-[bis(carboxymethyl)]amino-6-methylperhydro-1,4-diazepine (AAZTA), 5-(8-methyl-3,6,10,13,16,19-hexaaza-bicyclo[6.6.6]icosan-1-ylamino)-5-oxopentanoic acid (MeCOSar), 2-S-(4-isothiocyanatobenzyl)-1,4,7-triazacyclononane-1,4,7-triacetic acid (p-SCN-Bn)-NOTA, 1,4,7-triazacyclononane-1,4,7-triacetic acid (NOTA), N,N′-bis-[2-hydroxy-5-(carboxyethyl)benzyl]ethylenediamine-N,N′-diacetic acid) (HBED-CC), tris(hydroxypyridinone) (THP), MAS₃, and desferoxamine (DFO).

Additional chelators are also described in Price and Ovig (Chem. Soc. Rev., 2014, 43, 260-290), Boros and Packard (Chem. Rev. 2019, 119, 2, 870-901), Barndt et al. (1. Nucl. Med., 2018, 59, 1500-1506), and Sneddon and Cornelissen (Curr. Opin. Chem. Biol., 2021, 63, 152-162) (each of which is incorporated by reference in its entirely).

In some embodiments, the chelator is attached to the nanoparticle core through a chemical moiety that contains a primary amine, secondary amine, an amide, a thioester, or a disulfide bond. Additional chemical moieties that can be used to covalently link a chelator to a nanoparticle core are known in the art.

A variety of different methods can be used to covalently link a chelator to a nanoparticle core. In some embodiments, the fluorophore is attached to the nanoparticle core through reaction of: an amine group (present in the chelator or on the nanoparticle core) with an active ester, carboxylate, isothiocyanate, or hydrazine (e.g., present in the chelator or on the nanoparticle core); through reaction of a carboxyl group (e.g., present in the chelator or on the nanoparticle core) in the presence of a carbodiimide; through reaction of a thiol (e.g., present in the chelator or on the nanoparticle core) in the presence of maleimide; through the reaction of a thiol (e.g., present in the chelator or on the nanoparticle core) in the presence of maleimide or acetyl bromide; or through the reaction of an azide (e.g., present in the chelator or on the nanoparticle core) in the presence of glutaraldehyde. Additional methods for attaching a chelator to a nanoparticle core are known in the art.

In some embodiments, the nanoparticle core does not include a chelator. In some embodiments, the nanoparticle can include a radiolabel that is associated (e.g., covalently-linked) directly with the nanoparticle core.

Radiolabels

In some embodiments, the nanoparticles described herein comprise a radiolabel. In some embodiments, the radiolabel can be associated with the nanoparticle core. For example, in some embodiments, the radiolabel can be covalently or non-covalently bonded to the nanoparticle core via a linker. In some embodiments, the radiolabel can be covalently or non-covalently bonded directly to the nanoparticle core. In some embodiments, the radiolabel can be associated with the nanoparticle core or a composition or moiety surrounding the nanoparticle core e.g., via van der Waals forces). In some embodiments, the radiolabel can be associated with the nanoparticle core without a chelator (e.g., wherein the nanoparticle core is chelator-free). In some embodiments, the radiolabel can be associated with the nanoparticle core with a chelator. In some embodiments, the radiolabel forms a stable complex with a chelator.

In some embodiments, the nanoparticle core comprises about 1 radiolabel atom to about 15 radiolabel atoms (e.g., about 1 radiolabel atom to about 11 radiolabel atoms, about 1 radiolabel atom to about 12 radiolabel atoms, about 1 radiolabel atom to about 13 radiolabel atoms, about 1 radiolabel atom to about 14 radiolabel atoms, about 1 radiolabel atom to about 15 radiolabel atoms, about 13 radiolabel atoms to about 14 radiolabel atoms, about 13 radiolabel atoms to about 15 radiolabel atoms) per nanoparticle core. In some embodiments, the nanoparticle core comprises about 14 radiolabel atoms associated e.g., via a chelator) with each nanoparticle core.

In some embodiments, the radiolabel has an emission energy (e.g., 0⁺ energy) ranging from about 550 kiloelectron volts (keV) to about 3500 keV (e.g., about 550 keV to about 580 keV, about 550 keV to about 640 keV, about 550 keV to about 660 keV, about 550 keV to about 770 keV, about 550 keV to about 910 keV, about 579 keV to about 1200 keV, about 579 keV to about 1900 keV, about 579 keV to about 3500 keV). In some embodiments, the radiolabel has an emission energy of about 656 keV. In some embodiments, the radiolabel has an emission energy comparable (e.g. ±25 key) to the emission energy of fluorine-18 (F-18). In some embodiments, the radiolabel has an emission energy of about 656 keV.

In some embodiments, the radiolabel has a half-life that enables an adequate assessment of the slow pharmacokinetics of macromolecules and/or blood-pool agents. In some embodiments, the radiolabel has a half-life ranging from about 10 minutes to about 80 hours (e.g., about 10 minutes to about 13 hours, about 70 minutes to about 13 hours, about 110 minutes to about 13 hours, about 13 hours to about 26 hours, about 13 hours to about 80 hours). In some embodiments, the radiolabel has a half-life of about 12.7 hours.

A variety of different radiolabels that can be associated (e.g., covalently-linked or non-covalently-linked) to the nanoparticle core are known in the art. The radiolabel may be an alpha emitter, a beta emitter or a gamma emitter. In certain embodiments the radiolabel may have dual energy properties. In further embodiments the radiolabel may be copper-64 (Cu-64), copper-67 (Cu-67), F-18, yttrium-90 (Y-90), scandium-44 (SC-44), cobalt-55 (co-55), niobium-90 (Nb-90), rhenium-186 (Re-186), rhenium-188 (Re-188), terbium-161 (Tb-161), lutetium-177 (Lu-177), bismuth-231 (Bi-213), lead-212 (Pb-212), actinium-225 (Ac-225), zirconium-89 (Zr), or any combination thereof.

### Methods of Treatment

In certain embodiments, the present disclosure relates to methods for slowing growth of tumors (primary and/or secondary tumors) in a subject in need thereof comprising administering a composition or pharmaceutical formulation comprising an effective amount of a RIG-1 agonist precursor comprising a single-stranded 5′ uncapped triphosphate antisense oligonucleotide having a sequence complementary to an endogenous miRNA. In certain embodiments, the method further comprises administering a single stranded oligonucleotide sequence complementary to the single-stranded 5′ uncapped triphosphate antisense oligonucleotide. In embodiments, the methods comprise use of core/shell nanoparticles to deliver the RIG-I agonist precursor and a radiolabel.

In certain other embodiments, the present disclosure relates to methods for generating a localized immune response comprising administering to the subject a therapeutically effective amount of a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment thereby generating a localized immune response. In some embodiments, the disclosure contemplates methods for treating a solid tumor in a subject, the method comprising administering to the subject a therapeutically effective amount of a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment thereby generating a localized immune response. In some embodiments, the disclosure contemplates methods for detecting, diagnosing, and/or monitoring treatment of a solid tumor in a subject, the method comprising: administering to the subject a nanoparticle comprising a nanoparticle core; a radiolabel; and a single-stranded uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment thereby generating a localized immune response. In some embodiments, the RNA is mRNA. In some embodiments, the RNA is miRNA. In some embodiments, the miRNA is selected from the group consisting of SEQ ID NOs: 1-13.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated (e.g., a solid tumor). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure (e.g., a solid tumor) is achieved by administering one or more nanoparticles of the present disclosure in an “effective amount.” An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of one or more nanoparticles, in combination with one or more additional active agents or other supportive therapy for treating or preventing a disease or condition (e.g., a solid tumor). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more nanoparticles of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the nanoparticle of the present disclosure with the additional active agent or therapy and/or the desired effect.

The methods described herein include methods for treating a solid tumor in a subject, the method comprising administering to the subject a therapeutically effective amount of a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment thereby generating a localized immune response. Without wishing to be bound by theory, the nanoparticle delivers the single-stranded 5′ uncapped triphosphate or biphosphate modified RNA oligonucleotide to the solid tumor, wherein the single-stranded 5′ uncapped triphosphate or biphosphate modified RNA oligonucleotide forms a duplex with the tumor specific RNA thereby eliciting a tumor specific immune response via the RIG-I signaling pathway. As such, provided herein is a method of treating a solid tumor by combining RIG-I mediated immune activation against tumor cells while, optionally, inhibiting a miRNA or mRNA (e.g., use of a modified RNA oligonucleotide which is complementary to endogenous miR21). In some embodiments, the endogenously expressed mRNA or miRNA is oncogenic. In some embodiments, the endogenously expressed mRNA or miRNA is tumor-specific. As used herein, “tumor-specific RNA” refers to RNA (e.g., miRNA or mRNA) which is highly expressed in tumor cells as compared to non-tumor cells.

In vivo, miRNAs often exert regulatory functions within RNA-induced silencing complexes (RISCs). The core subunit of RISC is a miRNA bound to AGO2 (a member of the Argonaute family of proteins). The miRNA within the RISC complex comprises double-stranded miRNA, with one RNA strand being the miRNA-guide which guides the complex to the target mRNA, and the other RNA strand being the passenger strand, which is removed from the complex and degraded. AGO2 uses the miRNA-guide to identify a complementary target transcript for repression.

In some embodiments, the single-stranded 5′ uncapped triphosphate or biphosphate modified RNA oligonucleotide forms a duplex with the endogenous tumor-specific RNA. In some embodiments, the endogenous tumor-specific RNA is selected from miRNA or mRNA. In some embodiments, the miRNA or mRNA is oncogenic. In some embodiments, the single-stranded 5′ uncapped triphosphate or biphosphate modified RNA oligonucleotide forms a duplex with the miRNA. In some embodiments, the duplex is not cleaved by AGO2. In some embodiments, the duplex is released by AGO2. In some embodiments, the duplex comprises between 0-5 mismatched base pairs.

In some embodiments, the duplex activates RIG-I. In some embodiments, the RIG-I activation is at least 5%, 10%, 15% or 20% greater than activation by a corresponding unmodified monophosphate RNA oligonucleotide. In some embodiments, the RIG-I activation elicits a tumor specific immune response. In some embodiments, the single-stranded 5′ uncapped triphosphate or biphosphate modified RNA oligonucleotide competes with endogenous mRNA to bind the miRNA.

The methods disclosed herein include the treatment of disorders associated with abnormal apoptotic or differentiative processes, e.g., cellular proliferative disorders or cellular differentiative disorders, e.g., solid tumors. In certain embodiments, the methods are directed to a dual method of treatment comprising the combination of tumor-specific immune activation and inhibition of miRNA or mRNA. The methods can also be used to reduce the risk of developing disorders associated with abnormal apoptotic or differentiative processes, by triggering an immune response that targets developing cancer cells. In some embodiments, the disorder is a solid tumor, e.g., breast, prostate, pancreatic, brain, hepatic, lung, kidney, skin, or colon cancer. Generally, the methods include administering a therapeutically effective amount of a treatment as described herein, to a subject who is in need of, or who has been determined to be in need of, such treatment. In some embodiments, the methods include administering a therapeutically effective amount of a treatment comprising a modified RNA oligonucleotide, e.g., linked to a nanoparticle. In some embodiments, the nanoparticle is a magnetic nanoparticle.

As used in this context, to “treat” means to ameliorate at least one symptom of the disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes (e.g., cancer) will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting bladder, bone, lung, kidney, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Other types of cancers include, but are not limited to biliary tract cancer, brain cancer, breast cancer, cervical cancer, choriocarcinoma, colon cancer, endometrial cancer, esophageal cancer, gastric cancer, glioblastoma, intraepithelial neoplasm, liver cancer, lung cancer, melanoma, neuroblastoma, oral cancer, ovarian cancer, pancreatic cancer, prostate cancer, rectal cancer, sarcoma, skin cancer, stomach cancer, testicular cancer, thyroid cancer, uterine cancer, and renal cancer. In certain embodiments, the cancer is selected from malignant melanoma, squamous cell carcinoma, renal cell carcinoma, prostate carcinoma, bladder cell carcinoma, breast carcinoma, ovarian carcinoma, non-small cell lung cancer, small cell lung cancer, hepatocellular carcinoma, basaliom, colon carcinoma, cervical dysplasia, and Kaposi's sarcoma (AIDS-related and non-AIDS related).

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Treatment and Detection of Cancer

In some embodiments of any of the methods described herein, the nanoparticle is administered to a subject that has been diagnosed as having a cancer (e.g., having a primary solid tumor cancer or a metastatic solid tumor cancer). In some embodiments, the subject has breast cancer (e.g., a metastatic breast cancer). In some non-limiting embodiments, the subject is a man or a woman, an adult, an adolescent, or a child. In some embodiments, the subject has one or more symptoms of a cancer or metastatic cancer (e.g., a metastatic cancer in a lymph node). In some embodiments, the subject has severe or an advanced stage of cancer (e.g., a primary or metastatic cancer). In some embodiments, the subject has a metastatic tumor present in at least one lymph node. In some embodiments, the subject has undergone lymphectomy and/or mastectomy.

Metastatic cancer is a cancer that originates from a cancer cell from a primary tumor that has migrated to a different tissue in the subject. In some embodiments, the cancer cell from the primary tumor can migrate to a different tissue in the subject by traveling through the blood stream or the lymphatic system of the subject. In some embodiments, the metastatic cancer is a metastatic cancer present in a lymph node in a subject.

The symptoms of metastatic cancer experienced by a subject depend on the site of metastatic tumor formation. Non-limiting symptoms of metastatic cancer in the brain of a subject include: headaches, dizziness, and blurred vision. Non-limiting symptoms of metastatic cancer in the liver of a subject include: weight loss, fever, nausea, loss of appetite, abdominal pain, fluid in the abdomen (ascites), jaundice, and swelling of the legs. Non-limiting symptoms of metastatic cancer in the bone of a subject include: pain and bone breakage following minor or no injury. Non-limiting symptoms of metastatic cancer in the lung of a subject include: non-productive cough, cough producing bloody sputum, chest pain, and shortness of breath.

A metastatic cancer can be diagnosed in a subject by a health care professional (e.g., a physician, a physician's assistant, a nurse, or a laboratory technician) using methods known in the art. For example, a metastatic cancer can be diagnosed in a subject, in part, by the observation or detection of at least one symptom of a metastatic cancer in a subject (e.g., any of those symptoms listed above). A metastatic cancer can also be diagnosed in a subject using a variety of imaging techniques (e.g., alone or in combination with the observance of one or more symptoms of a metastatic cancer in a subject). For example, the presence of a metastatic cancer (e.g., a metastatic cancer in a lymph node) can be detected in a subject using computer tomography, magnetic resonance imaging, positron emission tomography, and X-ray. A metastatic cancer (e.g., a metastatic cancer in a lymph node) can also be diagnosed by performing a biopsy of tissue from the subject (e.g., a biopsy of a lymph node from the subject).

A metastatic tumor can form in a variety of different tissues in a subject, including, but not limited to: brain, lung, liver, bone, peritoneum, adrenal gland, skin, and muscle. The primary tumor can be of any cancer type, including but not limited to: breast, colon, kidney, lung, skin, ovarian, pancreatic, rectal, stomach, thyroid, or uterine cancer.

Any one or more of the nanoparticles described herein can be administered to a subject having a cancer (e.g., metastatic cancer), The one or more nanoparticles can be administered to a subject in a health care facility (e.g., in a hospital or a clinic) or in an assisted care facility. In some embodiments, the subject has been previously diagnosed as having a cancer (e.g., a primary cancer). In some embodiments, the subject has been previously diagnosed as having a metastatic cancer (e.g., a metastatic cancer in the lymph node). In some embodiments, the subject has already received therapeutic treatment for the primary cancer. In some embodiments, the primary tumor of the subject has been surgically removed prior to treatment with one of the nanoparticles described herein. In some embodiments, at least one lymph node has been removed from the subject prior to treatment with one of the nanoparticles described herein. In some embodiments, the subject may be in a period of cancer remission. In some embodiments, the subject is administered an additional supportive or adjunctive therapy. In some embodiments, the additional supportive or adjunctive therapy is a radiotherapy, cryotherapy, or ultrasound therapy. In some embodiments, the additional supportive or adjunctive therapy is surgery

In some embodiments, the administering of at least one nanoparticle results in a decrease (e.g., a significant or observable decrease) in the size of a metastatic tumor present in a lymph node, a stabilization of the size (e.g., no significant or observable change in size) of a metastatic tumor present in a lymph node, or a decrease (e.g., a detectable or observable decrease) in the rate of the growth of a metastatic tumor present in a lymph node in a subject. A health care professional can monitor the size and/or changes in the size of a metastatic tumor present in a lymph node in a subject using a variety of different imaging techniques, including but not limited to: computer tomography, magnetic resonance imaging, positron emission tomography, and X-ray. For example, the size of a metastatic tumor present in a lymph node of a subject can be determined before and after therapy in order to determine whether there has been a decrease or stabilization in the size of the metastatic tumor in the subject in response to therapy. The rate of growth of a metastatic tumor in the lymph node of a subject can be compared to the rate of growth of a metastatic tumor in another subject or population of subjects not receiving treatment or receiving a different treatment. A decrease in the rate of growth of a metastatic tumor in the lymph node of a subject can also be determined by comparing the rate of growth of a metastatic tumor in a lymph node both prior to and following a therapeutic treatment (e.g., treatment with any of the nanoparticles described herein). In some embodiments, the visualization of a metastatic tumor (e.g., a metastatic tumor in a lymph node) can be performed using imaging techniques that utilize a labeled probe or molecule that binds specifically to the cancer cells in the metastatic tumor (e.g., a labeled antibody that selectively binds to an epitope present on the surface of the primary cancer cell). In some embodiments, the visualization of a metastatic tumor (e.g., a metastatic tumor in a lymph node) can be performed using imaging techniques that utilize the radiolabel (e.g., copper-64) attached to the nanoparticle.

In some embodiments, the administering of at least one nanoparticle to the subject results in a decrease in the risk of developing an additional metastatic tumor in a subject already having at least one metastatic tumor (e.g., a subject already having a metastatic tumor in a lymph node) (e.g., as compared to the rate of developing an additional metastatic tumor in a subject already having a similar metastatic tumor but not receiving treatment or receiving an alternative treatment). A decrease in the risk of developing an additional metastatic tumor in a subject already having at least one metastatic tumor can also be compared to the risk of developing an additional metastatic tumor in a population of subjects receiving no therapy or an alternative form of cancer therapy.

In some embodiments, administering a nanoparticle to the subject decreases the risk of developing a metastatic cancer (e.g., a metastatic cancer in a lymph node) in a subject having (e.g., diagnosed as having) a primary cancer (e.g., a primary breast cancer) (e.g., as compared to the rate of developing a metastatic cancer in a subject having a similar primary cancer but not receiving treatment or receiving an alternative treatment). A decrease in the risk of developing a metastatic tumor in a subject having a primary cancer can also be compared to the rate of metastatic cancer formation in a population of subjects receiving no therapy or an alternative form of cancer therapy.

A health care professional can also assess the effectiveness of therapeutic treatment of a metastatic cancer (e.g., a metastatic cancer in a lymph node of a subject) by observing a decrease in the number of symptoms of metastatic cancer in the subject or by observing a decrease in the severity, frequency, and/or duration of one or more symptoms of a metastatic cancer in a subject. A variety of symptoms of a metastatic cancer are known in the art and are described herein. Non-limiting examples of symptoms of metastatic cancer in a lymph node include: pain in a lymph node, swelling in a lymph node, appetite loss, and weight loss.

In some embodiments, treatment with a nanoparticle disclosed herein increases (e.g., a significant increase) the chance of survival of a primary cancer or a metastatic cancer in a subject (e.g., as compared to a population of subjects having a similar primary cancer or a similar metastatic cancer but receiving a different therapeutic treatment or no therapeutic treatment). In some embodiments, treatment with a nanoparticle disclosed herein improves the prognosis for a subject having a primary cancer or a metastatic cancer (e.g., as compared to a population of subjects having a similar primary cancer or a similar metastatic cancer but receiving a different therapeutic treatment or no therapeutic treatment).

Methods of Decreasing Cancer Cell Invasion or Metastasis

Also provided are methods of decreasing (e.g., a significant or observable decrease) cancer cell invasion or metastasis in a subject that include administering at least one nanoparticle described herein to the subject in an amount sufficient to decrease cancer cell invasion or metastasis in a subject.

In some embodiments of these methods, the cancer cell metastasis is from a primary tumor e.g., any of the primary tumors described herein) to a secondary tissue (e.g., a lymph node) in a subject. In some embodiments of these methods, the cancer cell metastasis is from a lymph node to a secondary tissue e.g., any of the secondary tissues described herein) in the subject.

In some embodiments, the cancer cell invasion is the migration of a cancer cell into a tissue proximal to the primary tumor. In some embodiments, the cancer cell invasion is the migration of a cancer cell from a primary tumor into the lymphatic system. In some embodiments, the cancer cell invasion is the migration of a metastatic cancer cell present in the lymph node into the lymphatic system or the migration of a metastatic cancer cell present in a secondary tissue to an adjacent tissue in the subject.

Cancer cell invasion in a subject can be assessed or monitored by visualization using any of the imaging techniques described herein. For example, one or more tissues of a subject having a cancer or metastatic cancer can be visualized at two or more time points (e.g., at a time point shortly after diagnosis with a cancer and at later time point). In some embodiments, a decrease in cancer cell invasion in a subject can be detected by observing a decrease in the spread of a primary tumor through a specific tissue in the subject (when the spread of the primary tumor is assessed through the imaging techniques known in the art or described herein). In some embodiments, a decrease in cancer cell invasion can be detected by a reduction in the number of circulating primary cancer cells or circulating metastatic cancer cells in the blood or lymph of a subject.

Cancer cell metastasis can be detected using any of the methods described herein or known in the art. For example, successful reduction of cancer cell metastasis can be observed as a decrease in the rate of development of an additional metastatic tumor in a subject already having at least one metastatic tumor (e.g., a subject already having a metastatic tumor in a lymph node) (e.g., as compared to the rate of development of an additional metastatic tumor in a subject or a population of subjects already having a similar metastatic tumor but not receiving treatment or receiving an alternative treatment). Successful reduction of cancer cell metastasis can also be observed as a decrease in the risk of developing at least one metastatic cancer e.g., a metastatic cancer in a lymph node) in a subject having (e.g., diagnosed as having) a primary cancer (e.g., a primary breast cancer) (e.g., as compared to the risk of developing a metastatic cancer in a subject or a population of subjects having a similar primary cancer but not receiving treatment or receiving an alternative treatment).

Also provided herein are methods of detecting, diagnosing, and/or monitoring a cancer (e.g., metastatic cancer) in a subject that include administering any of the nanoparticles disclosed herein to the subject having the cancer (e.g., metastatic cancer) and imaging the nanoparticle.

In some embodiments of these methods, the nanoparticle is administered in an amount sufficient to image the nanoparticle in the subject. In some embodiments, the nanoparticle amount sufficient to detect, diagnose, and/or monitor a cancer metastatic cancer) in a subject is less than about 0.020 mg/kg (e.g., about 0.001 mg/kg to about 0.005 mg/kg, about 0.005 to about 0.010 mg/kg, about 0.010 mg/kg to about 0.015 mg/kg, about 0.011 mg/kg to about 0.015 mg/kg, about 0,012 mg/kg to about 0.015 mg/kg, about 0.013 mg/kg to about 0.015 mg/kg, about 0.012 mg/kg to about 0.016 mg/kg, about 0.013 mg/kg to about 0.017 mg/kg, about 0.013 mg/kg to about 0.018 mg/kg, or about 0.015 mg/kg to about 0.020 mg/kg). In some embodiments, the nanoparticle amount sufficient to detect, diagnose, and/or monitor a cancer (e.g., metastatic cancer) in a subject is less than about 0.001 mg/kg. In some embodiments, the nanoparticle amount sufficient to detect, diagnose, and/or monitor a cancer (e.g., metastatic cancer) in a subject is less than about 0.014 mg/kg. In some embodiments, the nanoparticle amount sufficient to detect, diagnose, and/or monitor a cancer (e.g., metastatic cancer) in a subject does not induce a drug side effect in the subject.

In some embodiments, the imaging is carried out using a non-invasive imaging technique. In some embodiments, the imaging is carried out using a minimally invasive imaging technique. As used herein, the term “minimally invasive imaging technique” comprises imaging techniques employing the use of an internal probe or injection of the composition provided herein via syringe. Example imaging techniques include, but are not limited to, magnetic resonance imaging (MRI), tomographic imaging, positron emission tomography (PET) imaging, single-photon emission computed tomography (SPECT) imaging, computed tomography (CT) imaging, PET with CT imaging, PET-MRI imaging, or any combination thereof. In some embodiments, the imaging is carried out by PET-MRI. In some embodiments, the nanoparticles of the disclosure can accumulate in cancer (e.g., metastatic cancer) tissue and thus, can be effective at highlighting cancer when imaged e.g., via PET or PET-MRI). In some embodiments, the subject is imaged to determine a location or number of tumor cells in the subject. In some embodiments, the subject is imaged to determine the location of the nanoparticles in the subject.

In some embodiments, the methods provided herein further comprise waiting a time sufficient to allow the nanoparticle to accumulate at a cell or tissue site (e.g., a cell or tissue site in a subject) associated with the cancer, prior to imaging. In some embodiments, the methods provided herein further comprise waiting a time sufficient to allow the nanoparticle to accumulate at a cell or tissue site (e.g., a cell or tissue site in a subject) associated with the cancer, prior to administering the dose of radiation to the subject. In some embodiments, the methods provided herein further comprise waiting a time sufficient to allow the nanoparticle to accumulate at a cell or tissue site (e.g., a cell or tissue site in a subject) associated with the cancer, prior to administering the dose of radiation to the subject and/or imaging the subject.

In some embodiments, the time sufficient to allow the nanoparticle to accumulate at a cell or tissue site is from about 30 seconds to about 24 hours, for example, about 30 seconds to about 24 hours, about 30 seconds to about 12 hours, about 30 seconds to about 6 hours, about 30 seconds to about 2 hours, about 30 seconds to about 1 hour, about 30 seconds to about 30 minutes, about 30 seconds to about 10 minutes, about 10 minutes to about 24 hours, about 10 minutes to about 12 hours, about 10 minutes to about 6 hours, about 10 minutes to about 2 hours, about 10 minutes to about 1 hour, about 10 minutes to about 30 minutes, about 30 minutes to about 24 hours, about 30 minutes to about 12 hours, about 30 minutes to about 6 hours, about 30 minutes to about 2 hours, about 30 minutes to about 1 hour, about 1 hour to about 24 hours, about 1 hour to about 12 hours, about 1 hour to about 6 hours, about 1 hour to about 2 hours, about 2 hours to about 24 hours, about 2 hours to about 12 hours, about 2 hours to about 6 hours, about 6 hours to about 24 hours, about 6 hours to about 12 hours, or about 12 hours to about 24 hours.

In some embodiments, the subject is administered one or more doses of radiation (e.g., ionizing radiation). In some embodiments, the radiation damages cells by inducing ionization and DNA damage. In some embodiments, the radiation may directly or indirectly trigger alterations in the expression levels of a miRNA (e.g., miR10b). In some embodiments, radiation of the nanoparticle may enhance the radiation damage effects to a localized area. In some embodiments, radiation of the nanoparticle may radiosensitize the cancer. In some embodiments, the radiation is external beam radiation therapy (EBRT).

RIG-I Receptor Activated Immune Response

As described previously, RIG-I is a cytosolic nucleic acid sensing Pattern Recognition Receptor (PRR) of the innate immune system. It is essential for recognizing RNA structures (like viruses) with a 5′ triphosphate signature. RIG-I activation can be programmed as an immune response against cancer. Importantly, tumor cell death through RIG-I has been shown to build immunological memory, meaning that once the body's immune system has been activated, the body becomes immune, and tumors are rejected as “foreign.”

In some embodiments, the disclosure contemplates methods for generating a localized immune response comprising administering to the subject a therapeutically effective amount of a nanoparticle comprising a nanoparticle core; a radiolabel; and a single-stranded 5′ uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment thereby generating a localized immune response. Without wishing to be bound by theory, the nanoparticle delivers the single-stranded 5′ uncapped triphosphate or biphosphate modified RNA oligonucleotide to the cancer cell. The immune system is selectively activated in the cancer cells when the single-stranded 5′ uncapped triphosphate or biphosphate modified RNA oligonucleotide forms a duplex with the tumor specific RNA thereby eliciting a tumor specific immune response via the RIG-I signaling pathway. In some embodiments, administration of the modified RNA oligonucleotide induces an anti-viral response, in particular, a type I IFN response. In some embodiments, the type I IFN response is an IFN-α response. In some embodiments, the RIG-I activation elicits a tumor specific immune response (e.g., a response against a tumor cell which highly expresses the tumor specific RNA). In some embodiments, the tumor specific immune response comprises release of type I IFNs, DAMPs (danger-associated molecular pattern), and/or tumor antigens. In some embodiments, the method induces immunological memory against said tumor cells. In some embodiments, the method increases the localized immune response to the cancer. In some embodiments, the method increases the localized immune response by at least 1 fold. In some embodiments, the method increases the localized immune response by at least 2 fold (e.g., at least 3 fold, 4 fold, 5 fold, 10 fold, 50 fold, or 100 fold). In some embodiments, the method increases the localized immune response by at least 3 fold. In some embodiments, the method increases the localized immune response by at least 4 fold. In some embodiments, the method increases the localized immune response by at least 5 fold. In some embodiments, the method increases the localized immune response by at least 6 fold. In some embodiments, the method increases the localized immune response by at least 7 fold. In some embodiments, the method increases the localized immune response by at least 8 fold. In some embodiments, the method increases the localized immune response by at least 9 fold. In some embodiments, the method increases the localized immune response by at least 10 fold. In some embodiments, the method increases the localized immune response by at least 15 fold. In some embodiments, the method increases the localized immune response by at least 20 fold. In some embodiments, the method increases the localized immune response by at least 30 fold. In some embodiments, the method increases the localized immune response by at least 40 fold. In some embodiments, the method increases the localized immune response by at least 50 fold. In some embodiments, the method increases the localized immune response by at least 60 fold. In some embodiments, the method increases the localized immune response by at least 70 fold. In some embodiments, the method increases the localized immune response by at least 80 fold. In some embodiments, the method increases the localized immune response by at least 90 fold. In some embodiments, the method increases the localized immune response by at least 100 fold.

In some embodiments, the administration of the nanoparticle comprising the modified RNA oligonucleotide induces apoptosis of a tumor cell. In some embodiments, the administration of the nanoparticle (a) induces an anti-viral response, in particular, a type I IFN response, and (b) downregulates a tumor-specific RNA (e.g., miRNA21) in a vertebrate animal, in particular, a mammal. The present application further provides the use of at least one nanoparticle for the preparation of a pharmaceutical composition for inducing apoptosis of a tumor cell in a vertebrate animal, in particular, a mammal.

Described herein are methods and/or compositions for eliciting a tumor specific immune response through the administration of nanoparticles thereby activating the body's immune system to effect the desired treatment response (e.g., treating and/or creating an anti-tumor immunological memory in an animal). Without wishing to be bound by theory, as shown in FIG. 1, the RIG-I pathway is selectively activated in cancer cells by in situ generation of 5′ppp-double strand RNA following introduction of 5′ppp single strand RNA oligonucleotide complementary to a miRNA or mRNA expressed specifically in these cells; the same or similar is expected from 5′pp-single strand RNA. Consequently, the antitumor immunity potential of the tumor microenvironment (TME) can be uncovered via the activation of RIG-I signaling pathway, in conjunction with concurrent activation of certain tumor suppressor gene(s), by simply using a single-stranded RNA. In some embodiments, the modified RNA oligonucleotide in FIG. 1 is delivered to the cancer using a radiolabeled nanoparticle.

### 6. Pharmaceutical Compositions & Modes of Administration

In embodiments provided herein, pharmaceutical compositions comprise an effective amount of a RIG-1 agonist precursor comprising a single-stranded 5′ uncapped triphosphate antisense oligonucleotide having a sequence complementary to an endogenous miRNA. In certain embodiments, the pharmaceutical compositions comprise a nanoparticle comprising a core-shell structure, wherein the shell comprises a RIG-1 agonist precursor comprising a single-stranded 5′ uncapped triphosphate antisense oligonucleotide having a sequence complementary to an endogenous miRNA. In certain embodiments, the nanoparticle comprises a radiolabel.

In certain embodiments, in any of the methods described herein, the nanoparticle comprising a nanoparticle core, a radiolabel, and a modified RNA oligonucleotide can be administered by a health care professional (e.g., a physician, a physician's assistant, a nurse, or a laboratory or clinic worker), the subject (i.e., self-administration), or a friend or family member of the subject. The administering can be performed in a clinical setting (e.g., at a clinic or a hospital), in an assisted living facility, or at a pharmacy.

In some embodiments, at least two (e.g., at least two, three, or four) of any of the types of nanoparticles described herein can be present in a pharmaceutical composition in any combination. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). In some embodiments, the compositions provided herein can include a pharmaceutically acceptable diluent (e.g., a sterile diluent). In some embodiments, the pharmaceutically acceptable diluent can be sterile water, sterile saline, a fixed oil, polyethylene glycol, glycerine, propylene glycol or other synthetic solvents, antibacterial or antifungal agents such as benzyl alcohol or methyl parabens, chlorobutanol, phenol, ascorbic acid, thimerosal, and the like, antioxidants such as ascorbic acid or sodium bisulfate, chelating agents such as ethylenediaminetetraacetic acid, buffers such as acetates, citrates, or phosphates, and isotonic agents such as sugars (e.g., dextrose), polyalcohols (e.g., mannitol or sorbitol), or salts (e.g., sodium chloride), or any combination thereof.

In some embodiments, the pharmaceutical compositions provided herein can include a pharmaceutically acceptable carrier. Liposomal suspensions can also be used as pharmaceutically acceptable carriers (see, e.g., U.S. Pat. No. 4,522,811).

Preparations of the compositions can be formulated and enclosed in ampules, disposable syringes, or multiple dose vials. Where required (as in, for example, injectable formulations), proper fluidity can be maintained by, for example, the use of a coating such as lecithin, or a surfactant. Absorption of the nanoparticles can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the nanoparticles described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage). In some embodiments, the compositions containing one or more of any of the nanoparticles described herein can be directly administered (injected) into a lymph node in a subject. In some embodiments, the compositions containing one or more of any of the nanoparticles described herein can be formulated into a dosage form that is an injectable, a tablet, a lyophilized powder, a suspension, or any combination thereof. In some embodiments, the compositions containing one or more of any of the nanoparticles described herein may be formulated for instant release, controlled release, timed-release, sustained release, extended release, or continuous release.

The pharmaceutical composition may be administered by any route known in the art, including, but not limited to, topical, enteral and parenteral routes, provided that it is compatible with the intended application. Topical administration includes, but is not limited to, epicutaneous, inhalational, intranasal, vaginal administration, enema, eye drops, and ear drops. Enteral administration includes, but is not limited to, oral, rectal administration and administration through feeding tubes. Parenteral administration includes, but is not limited to, intravenous, intraarterial, intramuscular, intracardiac, subcutaneous, intraosseous, intradermal, intrathecal, intraperitoneal, transdermal, transmucosal, and inhalational administration. The pharmaceutical composition may be use for prophylactic and/or therapeutic purposes.

In some embodiments, the subject is administered at least one (e.g., at least 2, 3, 4, 5, 6, 7, 8, 9, 10, 15, 20, 25, or 30) dose(s) of a composition comprising at least one (e.g., one, two, three, or four) of any of the nanoparticles described herein. In some embodiments of any of the methods described herein, the subject is administered at least one (e.g., at least 2, 3, 4, 5, 6, 7, 8, 9, 10, 15, 20, 25, or 30) dose(s) of a nanoparticle comprising at least one (e.g., one, two, three, or four) of any of the modified RNA oligonucleotides described herein.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) nanoparticles (e.g., any of the nanoparticles described herein) will be an amount that results in one of the following: (1) a decrease in cancer cell invasion or metastasis in a subject having cancer (e.g., breast cancer); (2) treatment of a metastatic cancer in a lymph node in a subject; (3) a decrease or stabilization of a metastatic tumor size in a lymph node in a subject; (4); a decrease in the rate of metastatic tumor growth in a lymph node in a subject; (5) a decrease in the severity, frequency, and/or duration of one or more symptoms of a metastatic cancer in a lymph node in a subject in a subject (e.g., a human); or (6) a decrease in the number of symptoms of a metastatic cancer in a lymph node in a subject e.g., as compared to a control subject having the same disease but not receiving treatment or a different treatment, or the same subject prior to treatment).

The effectiveness and dosing of any of the nanoparticles described herein can be determined by a health care professional using methods known in the art, as well as by the observation of one or more symptoms of a metastatic cancer in a lymph node in a subject (e.g., a human). Certain factors may influence the dosage and timing required to effectively treat a subject e.g., the severity of the disease or disorder, previous treatments, the general health and/or age of the subject, and the presence of other diseases).

Exemplary doses include milligram or microgram amounts of any of the nanoparticles described herein per kilogram of the subject's weight. For example, in some embodiments, the nanoparticles can be administered to a subject at a dose that is less than about 0.020 mg/kg (e.g., about 0.001 mg/kg to about 0.005 mg/kg, about 0.005 to about 0.010 mg/kg, about 0.010 mg/kg to about 0.015 mg/kg, about 0.011 mg/kg to about 0.015 mg/kg, about 0.012 mg/kg to about 0.015 mg/kg, about 0.013 mg/kg to about 0.015 mg/kg, about 0.012 mg/kg to about 0.016 mg/kg, about 0.013 mg/kg to about 0.017 mg/kg, about 0.013 mg/kg to about 0.018 mg/kg, or about 0.015 mg/kg to about 0,020 mg/kg). In some embodiments, the nanoparticles can be administered to a subject at a dose that is less than about 0.014 mg/kg. In some embodiments, the nanoparticles can be administered to a subject at a dose that is less than about 0.014 mg/kg for imaging, detecting, diagnosing, and/or monitoring a metastatic cancer tissue in a subject.

In some embodiments, the nanoparticles can be administered to a subject at a dose ranging from about 1 mg/kg to about 10 mg/kg (e.g., about 1 mg/kg to about 5 mg/kg, about 1 to about 7 mg/kg, about 2 mg/kg to about 7 mg/kg, about 2 mg/kg to about 8 mg/kg, about 2 mg/kg to about 9 mg/kg, about 2 mg/kg to about 10 mg/kg, about 3 mg/kg to about 5 mg/kg, about 3 mg/kg to about 6 mg/kg, about 3 mg/kg to about 7 mg/kg, about 3 mg/kg to about 8 mg/kg, about 3 mg/kg to about 9 mg/kg, about 3 mg/kg to about 10 mg/kg, about 4 mg/kg to about 5 mg/kg, about 4 mg/kg to about 6 mg/kg, about 4 mg/kg to about 7 mg/kg, about 4 mg/kg to about 8 mg/kg, about 4 mg/kg to about 9 mg/kg, about 4 mg/kg to about 10 mg/kg, about 5 mg/kg to about 6 mg/kg, about 5 mg/kg to about 7 mg/kg, about 5 mg/kg to about 8 mg/kg, about 5 mg/kg to about 9 mg/kg, about 5 mg/kg to about 10 mg/kg, about 6 mg/kg to about 7 mg/kg, about 6 mg/kg to about 8 mg/kg, about 6 mg/kg to about 9 mg/kg, about 6 mg/kg to about 10 mg/kg, about 7 mg/kg to about 8 mg/kg, about 7 mg/kg to about 9 mg/kg, or about 7 mg/kg to about 10 mg/kg). In some embodiments, the nanoparticles can be administered to a subject at a dose of about 5 mg/kg to about 7 mg/kg. In some embodiments, the nanoparticles can be administered to a subject at a dose that is less than about 5 mg/kg to about 7 mg/kg for treating a metastatic cancer and/or decreasing cell invasion or metastasis in a subject.

While these doses cover certain ranges, one of ordinary skill in the art will understand that therapeutic agents, including the nanoparticles described herein, vary in their potency, and effective amounts can be determined by methods known in the art. Typically, relatively low doses are administered at first, and the attending health care professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the nanoparticles in vivo.

In some embodiments, the nanoparticles are administered to a subject once a day. In some embodiments, the nanoparticles are administered to a subject twice a day. In some embodiments, the nanoparticles are administered to a subject once a week. In some embodiments, the nanoparticles are administered to a subject twice a week. In some embodiments, the nanoparticles are administered to a subject three times a week. In some embodiments, the nanoparticles are administered to a subject every two weeks. In some embodiments, the nanoparticles are administered to a subject every three weeks. In some embodiments, the nanoparticles are administered to a subject every four weeks. In some embodiments, the nanoparticles are administered to a subject every month.

The pharmaceutical compositions can be included in a kit, container, pack, or dispenser together with instructions for administration.

In some embodiments, the pharmaceutical composition further comprises an agent which facilitates the delivery of the nanoparticle into a cell, in particular, into the cytosol of the cell. In some embodiments, the delivery agent is a micelle, lipid nanoparticle (LNP), spherical nucleic acid (SNA), extracellular vesicle, synthetic vesicle, exosome, lipidoid, liposome, or lipoplex.

In some embodiments, the pharmaceutical composition may further comprise another agent such as an agent that stabilizes the modified RNA oligonucleotide of the nanoparticle. Examples of a stabilizing agent include a protein that complexes with the modified RNA oligonucleotide to form an iRNP, chelators such as EDTA, salts, and RNase inhibitors.

In some embodiments, the pharmaceutical composition, in particular, the pharmaceutical composition comprising a nanoparticle as described herein, further comprises one or more pharmaceutically active therapeutic agent(s). Examples of a pharmaceutically active agent include immunostimulatory agents, anti-viral agents, antibiotics, anti-fungal agents, anti-parasitic agents, anti-tumor agents, cytokines, chemokines, growth factors, anti-angiogenic factors, chemotherapeutic agents, antibodies and gene silencing agents. Preferably, the pharmaceutically active agent is selected from the group consisting of an immunostimulatory agent, an anti-viral agent and an anti-tumor agent. The more than one pharmaceutically active agents may be of the same or different category.

In some embodiments, the pharmaceutical composition, in particular, the pharmaceutical composition comprising a nanoparticle as described herein, further comprises an antigen, an anti-viral vaccine, an anti-bacterial vaccine, and/or an anti-tumor vaccine, wherein the vaccine can be prophylactic and/or therapeutic.

In some embodiments, the pharmaceutical composition, in particular, the pharmaceutical composition comprising a nanoparticle as described herein, further comprises retinoid acid, IFN-α and/or IFN-β. Without being bound by any theory, retinoid acid, IFN-α and/or IFN-β are capable of sensitizing cells for IFN-α production, possibly through the upregulation of RIG-I expression.

In some embodiments, the pharmaceutical composition, in particular, the pharmaceutical composition comprising a nanoparticle as described herein, further comprises at least one additional therapeutic agent. In some embodiments, the at least one additional therapeutic agent can be a chemotherapeutic agent (e.g., cyclophosphamide, mechlorethamine, chlorambucil, melphalan, daunorubicin, doxorubicin, epirubicin, idarubicin, mitoxantrone, valrubicin, paclitaxel, docetaxel, etoposide, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, bortezomib, carfilzomib, salinosporamide A, all-trans retinoic acid, vinblastine, vincristine, vindesine, and vinorelbine) and/or an analgesic (e.g., acetaminophen, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, phenylbutazone, piroxicam, sulindac, tolmetin, celecoxib, buprenorphine, butorphanol, codeine, hydrocodone, hydromorphone, levorphanol, meperidine, methadone, morphine, nalbuphine, oxycodone, oxymorphone, pentazocine, propoxyphene, and tramadol).

In some embodiments, the at least one additional therapeutic agent is an immunogenic cell death inducer (ICDi) (e.g., Daunorubicin, Docetaxel, Doxorubicin, Mitoxanthrone, Oxaliplatin, and Paclitaxel). In some embodiments, the at least one additional therapeutic agent is a siRNA therapy. In some embodiments, the siRNA therapy targets a gene associated with cancer (e.g., PD-L1, CTLA-4, TGF-β, and/or VEGF).

In some embodiments, the at least one additional therapeutic agent is a targeted therapy. Targeted therapies are a cornerstone of what is also referred to as precision medicine, a form of medicine that uses information about a person's genes and proteins to prevent, diagnose, and treat disease. Such therapeutics are sometimes called “molecularly targeted drugs,” or similar names. The process of discovering them is often referred to as “rational drug design.” This concept can also be referred to as “personalized medicine.”

Molecularly targeted drugs interact with a particular target molecule, or structurally related set of target molecules, in a pathway; thus modulating the endpoint effect of that pathway, such as a disease-related process; and, thus, yielding a therapeutic benefit.

Molecularly targeted drugs may be small molecules or biologics, usually antibodies. They may be useful alone or in combinations with other therapeutic agents and methods.

Because they target a particular molecule, or related set of molecules, and are usually designed to minimize their interactions with other molecules, targeted therapeutics may have fewer adverse side effects. Targeted cancer drugs block the growth and spread of cancer by interacting with specific molecules or sets of structurally related molecules (altogether, “molecular targets”) that are involved, broadly speaking, in the growth, progression, lack of suppression or elimination, or spread of cancer. Such molecular targets may include proteins or genes involved in one or more cellular functions including, for example and without limitation, signal transduction, gene expression modulation, apoptosis induction or suppression, angiogenesis inhibition, or immune system modulation.

Targeted therapy monoclonal antibodies (mAbs) and targeted small molecules are being used as treatments for cancer. They are used either as a monotherapy or in combination with other conventional therapeutic modalities, particularly if the disease under treatment is refractory to therapy using solely conventional techniques. In some embodiments, the at least one additional therapeutic agent is a molecularly targeted therapy. In some embodiments, the molecularly targeted therapy is selected from the group consisting of trastuzumab, gilotrif, proleukin, alectinib, campath, atezolizumab, avelumab, axitinib, belimumab, belinostat, bevacizumab, velcade, canakinumab, ceritinib, cetuximab, crizotinib, dabrafenib, daratumumab, dasatinib, denosumab, elotuzumab, enasidenib, erlotinib, gefitinib, ibrutinib, zydelig, imatinib, lenvatinib, midostaurin, necitumumab, niraparib, obinutuzumab, osimertinib, panitumumab, regorafenib, rituximab, ruxolitinib, sorafenib, tocilizumab, and trastuzumab.

In some embodiments, the at least one additional therapeutic agent is an immunotherapy. The term “immunotherapy,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non cytokine adjuvants. Alternatively, the immunotherapeutic treatment may consist of administering the subject with an amount of immune cells (T cells, NK, cells, dendritic cells, B cells, etc.).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

In some embodiments, the immunotherapy is selected from the group consisting of pembrolizumab (Keytruda®), nivolumab (Opdivo®), atezolizumab (Tecentriq®), ipilimumab (Yervoy®), avelumab (Bavencio®) and durvalumab (Imfinzi®). In some embodiments, the subject has undergone or is undergoing an anti-PD-1, anti-PD-L1, or anti-CTLA4 therapy. Alternatively, any of the methods may further comprise administering to the subject an effective amount of an anti-PD-1, anti-PD-L1, or anti-CTLA4 therapy. In some examples, the anti-PD-1, anti-PD-L1, or anti-CTLA4 therapy may comprise an anti-PD-1, anti-PD-L1, or anti-CTLA4 antibody, respectively. Exemplary anti-PD-1 antibodies include pembrolizumab, nivolumab, and AMP-224, or an antigen-binding fragment thereof. Exemplary anti-CTLA-4 antibodies include ipilimumab, and tremelimumab, or an antigen-binding fragment thereof. Exemplary anti-PD-L1 antibodies include durvalumab, atezolizumab, and avelumab, or an antigen-binding fragment thereof.

In some embodiments, at least one additional therapeutic agent and at least one nanoparticle are administered in the same composition (e.g., the same pharmaceutical composition). In some embodiments, the at least one additional therapeutic agent and the at least one nanoparticle are administered to the subject using different routes of administration (e.g., at least one additional therapeutic agent delivered by oral administration and at least one nanoparticle delivered by intravenous administration). By “at least one”, it is meant that one or more nanoparticles of the same or different nanoparticle(s) can be used together.

In some embodiments, the at least one additional therapeutic agent can be administered to the subject prior to administering the at least one nanoparticle (e.g., any of the nanoparticles described herein). In some embodiments, the one or more additional therapeutic agents can be administered to the subject after administering the at least one nanoparticle. In some embodiments, the one or more additional therapeutic agents and the at least one nanoparticle are administered to the subject such that there is an overlap in the bioactive period of the one or more additional therapeutic agents and the at least one nanoparticle in the subject.

In some embodiments, the subject can be administered the at least one nanoparticle or pharmaceutical composition (e.g., any of the nanoparticles or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or 10 years). A skilled medical professional may determine the length of the treatment period using any of the methods described herein for diagnosing or following the effectiveness of treatment (e.g., using the methods above and those known in the art). As described herein, a skilled medical professional can also change the identity and number (e.g., increase or decrease) of nanoparticles (and/or one or more additional therapeutic agents) administered to the subject and can also adjust (e.g., increase or decrease) the dosage or frequency of administration of at least one nanoparticle (and/or one or more additional therapeutic agents) to the subject based on an assessment of the effectiveness of the treatment (e.g., using any of the methods described herein and known in the art). A skilled medical professional can further determine when to discontinue treatment (e.g., for example, when the subject's symptoms are significantly decreased).

### 7. Methods of Preparing the Nanoparticles

Also provided herein are methods of preparing any of the nanoparticles of the disclosure that include preparing the nanoparticle core e.g., via any one of the methods described elsewhere herein), covalently linking the nucleic acid molecule to the nanoparticle core (NC); covalently linking the chelator to the nanoparticle core (e.g., a magnetic nanoparticle core) by reacting the nanoparticle core with the chelator at a ratio of about 40:1 equivalents (eq.) (i.e., 40 equivalents of the chelator to 1 equivalent of the nanoparticle core), adding a solution of ⁶⁴CuCh to the nanoparticle core, and purifying a mixture of the solution of ⁶⁴CuCh and the nanoparticle core to yield the nanoparticle.

In some embodiments, the methods include reacting the nanoparticle core with the chelator at a ratio ranging from about 5:1 chelator eq. :NC to about 60:1 chelator eq.:NC (e.g., about 5:1 chelator eq.:NC to about 10:1 chelator eq.:NC, about 5:1 chelator eq.:NC to about 12:1 chelator eq.:NC, about 5:1 chelator eq.:NC to about 15:1 chelator eq.:NC, about 1 chelator eq.:NC to about 20:1 chelator eq.:NC, about 5:1 chelator eq.:NC to about 25:1 chelator eq.:NC, about 5:1 chelator eq.:NC to about 30:1 chelator eq.:NC, about 5:1 chelator eq.:NC to about 35:1 chelator eq.:NC, about 5:1 chelator eq.:NC to about 40:1 chelator eq.:NC, about 5:1 chelator eq. :NC to about 45:1 chelator eq.:NC, about 5:1 chelator eq.:NC to about 50:1 chelator eq.:NC, about 5:1 chelator eq.:NC to about 55:1 chelator eq.:NC, or about 5:1 chelator eq.:NC to about 60:1 chelator eq.:NC, about 10:1 chelator eq.:NC to about 12:1 chelator eq.:NC, about 10:1 chelator eq.:NC to about 15:1 chelator eq.:NC, about 10:1 chelator eq.:NC to about 20:1 chelator eq.:NC, about 10:1 chelator eq.:NC to about 25:1 chelator eq.:NC, about 10:1 chelator eq.:NC to about 30:1 chelator eq.:NC, about 10:1 chelator eq.:NC to about 35:1 chelator eq.:NC, about 10:1 chelator eq.:NC to about 40:1 chelator eq.:NC, about 10:1 chelator eq.:NC to about 45:1 chelator eq.:NC, about 10:1 chelator eq.:NC to about 50:1 chelator eq.:NC, about 10:1 chelator eq.:NC to about 55:1 chelator eq.:NC, or about 10:1 chelator eq.:NC to about 60:1 chelator eq.:NC, about 15:1 chelator eq.:NC to about 20:1 chelator eq.:NC, about 15:1 chelator eq.:NC to about 25:1 chelator eq.:NC, about 15:1 chelator eq.:NC to about 30:1 chelator eq.:NC, about 15:1 chelator eq.:NC to about 35:1 chelator eq.:NC, about 15:1 chelator eq.:NC to about 40:1 chelator eq.:NC, about 15:1 chelator eq.:NC to about 45:1 chelator eq.:NC, about 15:1 chelator eq.:NC to about 50:1 chelator eq.:NC, about 15:1 chelator eq.:NC to about 55:1 chelator eq.:NC, or about 15:1 chelator eq.:NC to about 60:1 chelator eq.:NC, about 20:1 chelator eq.:NC to about 45:1 chelator eq.:NC, about 25:1 chelator eq.:NC to about 45:1 chelator eq.:NC, about 30:1 chelator eq.:NC to about 45:1 chelator eq.:NC, about 35:1 chelator eq.:NC to about 45:1 chelator eq.:NC, about 40:1 chelator eq.:NC to about 45:1 chelator eq.:NC, about 35:1 chelator eq.:NC to about 50:1 chelator eq.:NC, about 38:1 chelator eq.:NC to about 40:1 chelator eq.:NC, about 38:1 chelator eq.:NC to about 42:1 chelator eq.:NC, about 38:1 chelator eq.:NC to about 45:1 chelator eq.:NC, about 40:1 chelator eq.:NC to about 50:1 chelator eq.:NC, about 40:1 chelator eq.:NC to about 55:1 chelator eq.:NC_ or about 40:1 chelator eq.:NC to about 60; 1 chelator eq.:NC).

In some embodiments, covalently linking the chelator to the nanoparticle core is performed at a temperature of about 0° C. to about 8° C. (e.g., about 0° C. to about 4° C., about 1° C. to about 4° C., about 2° C. to about 4° C., about 3° C. to about 4° C., about 2° C. to about 6° C., about 3° C. to about 7° C., about 4° C. to about 5° C., about 4° C. to about 6° C., about 4° C. to about 7° C., or about 4° C. to about 8° C.). In some embodiments, covalently linking the chelator to the nanoparticle core is performed at a temperature of about 4° C.

In some embodiments, these methods further include heating the mixture of the solution of ⁶⁴CuCh and the nanoparticle core at a temperature of about 40° C. to about 65° C. (e.g., about 40° C. to about 65° C., about 45° C. to about 65° C., about 50° C. to about 65° C., about 55° C. to about 65° C., about 56° C. to about 65° C. about 57° C. to about 65° C., about 58° C. to about 65° C., about 59° C. to about 65° C., about 58° C. to about 62° C., about 59° C. to about 61° C., about 58° C. to about 63° C., about 59° C. to about 63° C., about 59° C. to about 60° C., about 60° C. to about 61° C., or about 60° C. to about 62° C.). In some embodiments, these methods further include heating the mixture of the solution of ⁶⁴CuCh and the nanoparticle core at a temperature of about 60° C.

In some embodiments, these methods further include heating the mixture of the solution of ⁶⁴CuCh and the nanoparticle core for about 10 min. to about 30 min. (e.g., about 10 min. to about 20 min., about 11 min. to about 21 min., about 12 min. to about 22 min., about 13 min. to about 23 min., about 14 min. to about 24 min., about 15 min. to about 25 min., about 16 min. to about 26 min., about 13 min. to about 20 min., about 15 min. to about 20 min., about 15 min. to about 2.2 min., about 18 min. to about 20 min., about 18 min. to about 22 min., about 19 min. to about 21 min., about 19 min. to about 23 min., about 20 min. to about 25 min., or about 20 min. to about 30 min.), in some embodiments, these methods further include heating the mixture of the solution of ⁶⁴CuCh and the nanoparticle core for about 20 min.

In some embodiments, these methods do not subject the nanoparticles to temperatures exceeding about 65° C. In some embodiments, these methods do not subject the nanoparticles to temperatures exceeding about 60° C. In some embodiments, the methods disclosed herein include the use of a chelator to associate the radiolabel with the nanoparticle core to advantageously avoid any harsh conditions (e.g., temperatures exceeding about 60° C. for longer than about 20 minutes) that may potentially damage the nucleic acid molecule.

The abbreviations used herein, including the Examples, are shown below:RIGA: RIG-I agonist

miR/miRNA: small, evolutionary conserved, single-stranded, non-coding RNA molecules that bind target mRNA to prevent protein production by one of two distinct mechanisms.

anti-miR/anti-miRNA: anti-miRNA oligonucleotides (AMOs) that bind miRNAs through complementary sequences and inhibit miRNA functions in living cells.

miR-21 mimic: synthetic miRNA-21, also used exchangeable with miR-21/miRNA-21.

RIGA-miR-21: 5′-ppp-anti-miRNA-21

anti-miR-21: anti-miRNA-21

TTX-RIGA-miR-21: TTX-5′-ppp-anti-miR-21

TTX-miR-21: TTX-anti-miR-21

ppp-dsRNA/5′-ppp-dsRNA: 5′-ppp-double—stranded RNA, a commercial RIG-I agonist.

### EXAMPLES

The invention now being generally described, it will be more readily understood by reference to the following examples, which are included merely for purposes of illustration of certain embodiments of the present invention, and are not intended to limit the invention.

### Example 1—Materials and Methods

Oligonucleotides

The miRNA-21 mimic, 5′-UAGCUUAUCAGACUGAUGUUGA-3′, (SEQ ID NO: 19) and its 100% complement sequence, [+/−(ppp)]-5′-UCAACAUCAGUCUGAUAAGCUA-3′-(s-s) (SEQ ID NO: 6) were synthesized by Eurogentec North America (Fremont, CA). 5′-Triphosphate modification (ppp) is required for RIG-I agonism and 3′ disulfide bond (s-s) modification allows the oligos to be covalently conjugated to the nanoparticles. A 19-mer 5′ppp-dsRNA positive control for RIG-I activation was obtained from InvivoGen (Catalog No. tlrl-3prna, San Diego, CA).

Analytical Methods

Dynamic light Scattering (DLS) and Electrophoretic Light Scattering (ELS). DLS and ELS measurements were performed on Zetasizer Lab (Malvern Panalytical, UK) and accessories with samples being diluted to 0.1 mg Fe/m1, in dH₂O.

Colorimetric Determination of Iron using 0-Phenanthroline (OPT Iron Assay). The iron assay was used for the determination of nanoparticle concentration expressed as iron concentration. Ten (10) microliters of test sample were incubated with 90 μL of 6 N HCl at RT for 10 min. Twenty (20) μL of the digestion mixture was mixed with 20 μL of 0.4% ascorbic acid (w/v), incubated at RT for 10 min, followed by addition of 130 μL 1 M NaOAc and 200 μL 0.8% o-phenanthroline (w/v) in 50% ethanol (v/v). After 5-10 min at RT, the samples were diluted with 6304 of dH₂O, transferred (3×250 μL) into a microplate and read at 510 nm using the microplate reader.

Quantification of Amine Groups on Nanoparticles. The surface amine groups on the nanoparticles were determined indirectly by functionalization of TTX-NH₂ (2 mg Fe) with excess amount of heterobifunctional linker (2 mg SPDP/mg Fe), succinimidyl 3-(2-pyridyldithio)propionate (SPDP), in 33% DMSO for 1 h at RT. After purification by ultracentrifugation, the resulting particle (TTX-PDP) was diluted to 1.0 mg Fe/mL in PBS and equal volumes were distributed to 2 microtubes, which was treated with 1.5% TCEP (pH 7) (1:5) (T—Test) or PBS (C—Control). After 30 min at RT, the mixtures were transferred to separate Amicon Ultra-0.5 mL filters and centrifuged for 10 min at 14,000×g, 4° C. Equal volumes of filtrates were treated with PBS (T) or 3% TCEP (C) at a ratio of 1:5 for 10 min. Then the reaction mixtures were measured at 343 nm on NanoDrop spectrophotometer to quantify released 2-MP. The differential reading between T and C reflected the contribution of PDP linkers and was used to calculate the number of PDP linkers on the NPs.

Determination of Oligos in Nanodrugs. The nanodrug sample was diluted to 1.0 mg Fe/mL and distributed into 2×1.5 mL microtubes. Exactly 0.1 vol PBS was added to Tube A, 0.1 vol. of 3% TCEP·HCl to Tube B. After 30 min at RT, 1 vol. of 1 M NH₄HCO₃ was added to Tube B. The final samples were processed by ultrafiltration and the resulting filtrates were analyzed using NanoDrop Spectrophotometer with the wavelength specified at 260 nm and 343 nm.

Synthesis of TTX-RIGA-miR-21 (“RIG-I Agonist Precursor”) and TTX-miR-21

Dextran-coated iron oxide nanoparticles (TTX) were synthesized essentially following a published protocol. Briefly, dextran T10 solution (9.0 g in 30 mL dH₂O) was premixed with FeCl₃·6H₂O solution (0.65 g in 1.0 mL dH₂O), filtered (0.22 μm), cooled in an ice bath and flushed with N₂ for 30 min while stirring, followed by addition of FeCl₂·4H₂O solution (0.40 g in 1.0 mL dH₂O). The particle formation was initiated by the addition of ice-cooled 28% (vol/vol) ammonia (14 mL). The particles were then heated and maintained at 70-85° C. for 90 min, followed by cooling to RT and purification by TFF ultrafiltration against dH₂O to ˜10 mg Fe/mL. The particles were treated with epichlorohydrin (16 mL, RT, 8 h) to stabilize the dextran layer via cross-linking and install epoxide anchors, followed by amination with NH 4 OH (35 mL, 48-60 h). After extensive ultrafiltration and buffer exchanged into PBS using TFF, the aminated NPs (TTX-NH 2) were sterile filtered and stored at 4° C. Functionalization of the particles was completed by incubating TTX-NH₂ with heterobifunctional SPDP (0.3 mg SPDP/mg Fe) in the presence of 33% DMSO (v/v) at 4° C. on a rocker platform for 16 h, followed by TFF to PBS (TTX-PDP). Activation of the protected thiol-modified oligos was performed by reduction with TCEP and purification by ethanol/ammonium acetate precipitation. Oligo conjugation was routinely performed at ≤5 mg Fe/mL by mixing equal volumes of TTX-PDP solution and the deprotected thiol-oligo solution at a desired molar ratio, followed by incubation at 2-8° C. on a rotator for 16-24 h, and purification by spin filtration. The final product was designated as TTX-RIGA-miR-21 for 5′-ppp-anti-miRNA-21 and TTX-miR21 for anti-miRNA-21.

Cell Culture

The RIG-I reporter cell line HEK-Lucia™ RIG-I cells (Catalog Code, hkl-hrigi, InvivoGen) and the control cell line HEK-Lucia™ Null cells (Catalog Code, hkl-null, InvivoGen), and mouse skin melanoma cells (B16-F10) were cultured in Dulbecco's Modified Eagles Medium (DMEM, Gibco). All culture media contained 10% fetal bovine serum (serum, Gibco), 100 U/mL of penicillin, and 100 μg/mL of streptomycin. Cells were incubated at 37° C. in 5% CO₂, 5% humidity, and passaged at 2×10⁴ cells/mL when near-confluent monolayers were achieved. Cells were free from Mycoplasma contamination. To generate B16-F10 miRNA-21 transfectants, mature miRNA-21 mimic (0, 0.3, 3, 30, 300, and 1000 ng/mL) was transfected into the cells using LyoVec cationic lipid-based transfection agent (Catalog Code, lyec-12, InvivoGen). Briefly, miRNA-21 mimic was mixed with 100 μL of LyoVec™. The mixture was incubated at 15-25° C. for 15 min to 1 h to allow the formation of the complex prior to use for transfection of cells. Ten μL of the LyoVec/miRNA-21 mimic complex was added to 200 μL of culture media.

Isolation of miRNA and Quantitative Real-Time PCR

After treatment, miRNA was purified from cells using miRNeasy Mini Kit (Catalog No. 217004, Qiagen) according to the protocol recommended by the manufacturer. Then, complementary DNA (cDNA) was synthesized using miRCURY LNA RT Kit (Catalog No. 339340, Qiagen). For each miRNA target, cDNA was diluted prior to use at the ratio of 1:10 with nuclease free water.

The expression analysis of miR-21 was executed by using the miRCURY LNA SYBR Green PCR Kit (Catalog No. 339345, Qiagen) and primers from miRCURY LNA miRNA PCR Assays (Catalog No. 339306, Qiagen) for hsa-miR-21-5p (GeneGlobe ID YP00204230). Reactions were run on StepOnePlus Real-Time PCR System (Applied Biosystems) using the following cycling program: 2 min at 95° C. and 2-step cycling (40 cycles) of denaturation (10 s at 95° C.), and combined annealing/extension (60 s at 56° C.).

The calculation of relative expression was performed using the 2^{-ΔΔct} method. U6 SNRNA (GeneGlobe ID YP02119464) was used as a reference.

Western Blotting

Cells (3×10⁵) were transfected with 1 μg/mL, 5′-ppp-dsRNA positive control (InvivoGen), RIGA-miR-21 (4 μg/mL), or anti-miR-21 (4 μg/mL) with or without miR-21 mimic (1 μg/mL), using LyoVec transfection agent (Catalog Code: lyec-12, InvivoGen). After 48 h incubation, the media were aspirated, and the cells were washed twice in ice cold 1×PBS and lysed directly in the plate in IP-lysis buffer (Catalog No. 87787, Pierce/Thermo Scientific) containing Halt Protease and phosphatase inhibitor cocktails (100×) (Catalog No. 78440, Thermo Scientific) for 15 min on ice. Lysates were then centrifuged at 14,000×g at 4° C. for 10 min. The supernatant was collected, and protein concentration was determined using the Quick Start Bradford Protein assay kit (Catalog No. 5000202, Bio-Rad). Equal amounts of the proteins (50 μg) were electrophoresed (125 volts, 1 h) on a 4-20% Mini protean TGX stain-free protein gel (Bio-Rad) and transferred onto nitrocellulose membranes (125 volts, 1 h). Membranes were blocked for 1 h at room temperature with Blocker Blotto in TBS (Catalog No. 37530, Thermo Scientific) and incubated at 4° C. overnight with primary antibodies in blocking buffer (1:1000 dilution), rabbit monoclonals to RIG-I (Catalog No. 3743), phospho-p65 (Catalog No. 3033), p65 (Catalog No. 8242) and β-actin (Catalog No. 5125) from Cell Signaling Technology, where β-actin served as the reference protein. The membranes were incubated further with anti-rabbit IgG (secondary antiserum), horseradish peroxidase-conjugated secondary antibodies (1:2000 dilution; Catalog No. 7074, Cell Signaling Technology) for 2 h at 37° C. Detection of immunoreactive bands was carried out using Pierce ECL plus Western blotting substrate (Catalog No. 32132, Thermo Scientific) and the IBRIGHT CL750 imaging system (Thermo Scientific) according to the manufacturer's instructions.

RIG-I Activation Assay

HEK-Lucia™ RIG-I or HEK-Lucia™ Null cells (1, 2, 2.5 or 5×10⁴ cells/well) were seeded in 96-well plates at 70-85% confluence. Then 5′-ppp-dsRNA positive control (1 μg/mL, InvivoGen) or RIGA-miRNA-21 (1, 2 or 4 μg/mL) were transfected into the cells using LyoVec™ (Catalog Code lyec-12, InvivoGen) following the manufacturer's instructions. The transfection was performed immediately after cell seeding. Cells were incubated for 48 h at 37° C. and 5% CO₂. Next, 20 μL of culture media was transferred to a 96-well clear-bottom black plate and 50 μL of QUANTI-Luc™ assay solution (Catalog Code rep-qlc, Invivogen) was added directly to each well. Luminescence was immediately measured using a Spectramax M3 microplate reader (Molecular Devices) set at a 0.1 second of exposure.

Cell Viability

Cell viability was determined using the CellTiter-Glo Luminescent Cell Viability Assay Kit (Catalog No. G7570, Promega) according to the manufacturer's instructions. Briefly, B16-F10 cells were plated in a 96-well clear bottom black plate (Corning, Tewksbury, MA) at a density of 2×10⁴ cells/well in culture media (DMEM supplemented with 10% FBS and penicillin/streptomycin). The 5′-ppp-dsRNA positive control (1 μg/mL, InvivoGen), RIGA-miR-21 (1, 2 or 4 μg/mL) or anti-miR-21 (1, 2 or 4 μg/mL) were transfected into the cells using LyoVec™ (Catalog Code lyec-12, InvivoGen) following the manufacturer's instructions. After 48 h incubation, 1004 of CellTiter-Glo (Catalog No. G9242, Promega) was added into each well (containing 1004 of culture media) and incubated for 10 min at room temperature to stabilize the luminescence signal. Luminescence signal was measured using a SpectraMax M3 microplate reader (Molecular Devices).

Caspase 3/7 Activation

Caspase 3/7 activity was determined using the Caspase-Glo 3/7 Assay Kit (Catalog No. G8091, Promega) according to the manufacturer's instructions. Briefly, B16-F10 cells (1 or 2×10⁴ cells/well) were seeded in 96-well plates (Corning, Tewksbury, MA) and treated with 5′-ppp-dsRNA positive control (1 μg/mL, InvivoGen), RIGA-miRNA-21 (1, 2 or 4 μg/ml) or anti-miR-21 (1, 2 or 4 μg/ml) for 48 h. Plates were allowed to equilibrate to room temperature. One hundred microliters of Caspase-Glo® 3/7 Reagent was added to each well. Plates were incubated at room temperature for 1 h. Luminescence was measured using a SpectraMax M3 microplate reader.

IFN-γ-Inducible Protein 10 (IP-10/CXCL-10) Immunoassay

B16-F10 cells were treated with RIGA-miRNA-21 and respective controls for 48 h. IP-10/CXCL10 release from mouse cells was assayed using the Quantikine Mouse IP-10 ELISA assay (Catalog No. DY466-05, R&D Systems) using cell-free supernatants of the stimulated cells according to the manufacturer's instructions. The absorbance was measured at 405 nm, and the concentration of IP-10 in the samples was determined by comparison to the standards.

Statistical Analysis

Data were expressed as mean±sem. Statistical comparisons were drawn using a two-tailed t-test or ANOVA, where indicated. Dose response was analyzed using non-linear regression. All statistical tests were performed using GraphPad Prism software. A P value of less than 0.05 was considered statistically significant.

### Example 2—Design of the Template-Specific RIG-I Agonist Precursor, 5′-Ppp-Anti-miRNA-21 (RIGA-miR-21)

In the present study, we chose miRNA-21 as the target for template-directed assembly of the RIG-I agonist, based on its demonstrated abundance in tumor cells (Bautista-Sanchez et al., 2020). However, the underlying concept is modular and can be applied to other miRNA targets that are enriched or otherwise highly expressed in tumor cells. The designed RIGA-miR-21 construct is 100% complementary to endogenous miRNA-21, modified with a 5′-ppp, and lacks any internal modifications (FIG. 1a). FIGS. 1b and 1c illustrate a working model for the proposed action of the RIG-I agonist precursor RIGA-miR-21. Upon cellular entry, RIGA-miR-21 hybridizes with endogenous miRNA-21, forming a 5′-ppp-dsRNA complex (“RIG-I agonist”), i.e., the standard RIG-I agonist (Yoo et al., 2014; Lima et al., 2018). This hybridization event likely results in the release of miRNA from the RNA-induced silencing complex (RISC), as previously observed (De et al., 2013). The 5′-ppp-dsRNA complex is then recognized by RIG-I in the cytosol, leading to its activation (FIG. 1c). Consistent with the known mechanism of immune modulation through RIG-I agonism (Thoresen et al., 2021), RIG-I signaling can stimulate the rapid production of type I interferons (IFNs) and induce direct cancer cell death, triggering the release of IFNs, pro-inflammatory cytokines, and tumor antigens (TAs), consequently promoting cell-mediated immunity (FIG. 1d). This remodeling of the tumor microenvironment (TME) energizes both innate immune cells and the adaptive immune system. Leukocytes such as natural killer (NK) cells and macrophages enhance their cytolytic activity in response to an IFN-rich environment. Importantly, the increase in IFNs and TAs can also activate the adaptive immune response, leading to the maturation and activation of macrophages and dendritic cells (DCs), and improved antigen presentation to T-lymphocytes in tumor-draining lymph nodes. Naïve T cells are subsequently activated, proliferated, differentiated, and recruited to the TME, where they exert an effective anti-tumor response through direct cytolytic activity mediated by perforin and granzymes, as well as indirect cytolytic activity through the secretion of cytokines such as IFN-γ and TNFα. Following cancer cell death, most tumor-specific effector CD8+ T cells undergo apoptosis, but some may persist and develop into long-lived protective memory CD8+ T cells (FIG. 1d).

### Example 3—the Template-Specific RIG-I Agonist Precursor, RIGA-miR-21, Effectively Agonizes RIG-I in a Template-Dependent Manner in the Reporter Cell Line

This study aimed to investigate the ability of RIGA-miR-21 to induce RIG-I activation in the human RIG-I luciferase reporter cell line, HEK-Lucia™ RIG-I. This commercially available cell line exhibits stable expression of high levels of human RIG-I and secreted Lucia luciferase reporter. The reporter gene is regulated by an IFN-inducible ISG54 promoter that is enhanced by the multimeric IFN-stimulated response element (ISRE). By monitoring the activity of the Lucia luciferase, HEK-Lucia™ RIG-I and HEK-Lucia™ Null control cells can be utilized to investigate the activation of RIG-I signaling.

We first confirmed the presence of miRNA-21 in both cell lines by RT-PCR (FIG. 2a), and the high expression of RIG-I in HEK-Lucia RIG-I reporter cells using Western blotting (FIG. 2b) We also validated the differential sensitivity of the HEK-Lucia™ RIG-I and HEK-Lucia™ Null control cells to a commercially available traditional RIG-I agonist, consisting of a 5′ triphosphate double-stranded RNA 19-mer (5′-ppp-dsRNA or ppp-dsRNA). There was a highly significant enhancement of luciferase activity in the RIG-I overexpressing cells, as compared to the null cells (FIG. 2c).

We next evaluated the capacity of our template-specific RIG-I agonist precursor, RIGA-miR-21, to activate RIG-I. HEK-Lucia™ RIG-I and HEK-Lucia™ Null control cells were treated with RIGA-miR-21. We observed significant RIG-I activation in the HEK-Lucia™ RIG-I but not the HEK-Lucia™ Null cells at all three dose levels of RIGA-miR-21tested (FIG. 2a), despite the equivalent expression of miR-21 in both cell lines (FIG. 2c). Given the strict requirement for the formation of an RNA duplex for RIG-I activation [Schmidt, A., Schwerd, T., Hamm, W., Hellmuth, J. C., Cui, S., Wenzel, M., Hoffmann, F. S., Michallet, M. C., Besch, R., Hopfner, K. P., Endres, S., and Rothenfusser, S. (2009). 5′-triphosphate RNA requires base-paired structures to activate antiviral signaling via RIG-I. Proc Natl Acad Sci USA 106, 12067-12072; Takahasi, K., Yoneyama, M., Nishihori, T., Hirai, R., Kumeta, H., Narita, R., Gale, M., Jr., Inagaki, F., and Fujita, T. (2008). Non-self RNA-sensing mechanism of RIG-I helicase and activation of antiviral immune responses. Mol Cell 29, 428-440; Marq, J. B., Kolakofsky, D., and Garcin, D. (2010). Unpaired 5′ ppp-nucleotides, as found in arenavirus double-stranded RNA panhandles, are not recognized by RIG-I. J Biol Chem 285, 18208-18216], these results provided support for a template-directed mechanism of RIG-I agonism.

To further investigate the template-dependence of the observed RIG-I activation when using our RIGA-miR-21, we repeated the reporter assay using HEK-Lucia™ RIG-I cells transiently transfected with increasing concentrations of a mature miRNA-21 mimic (miR-21 mimic or miR-21). Transfection with the mimic was carried out prior to addition of RIGA-miR-21 to avoid annealing between them prior to entry into the cell. We observed a highly significant induction of RIG-I signaling by our RIGA-miR-21 in cells transfected with 30 and 300 ng/mL of the mimic (FIG. 2e), even in cultures of as few as cells. The levels of activation with RIGA-miR-21 were similar to those observed with the commercially available ppp-dsRNA positive control (FIG. 2e). The 5′-ppp-deficient anti-miR-21 (anti-miR-21) failed to cause detectable RIG-I activation (FIG. 2e).

### Example 4—RIGA-miR-21 Agonizes RIG-I in a Template-Dependent Manner and Induce Apoptosis in Melanoma Cells

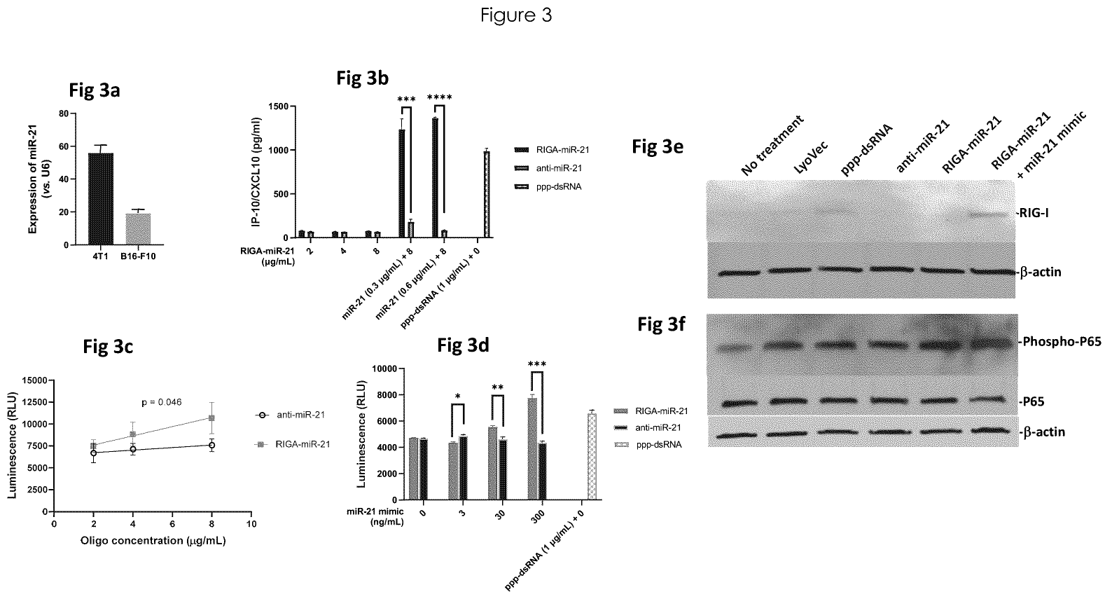

After successfully establishing the template-dependence of RIG-I activation by RIGA-miR-21 in RIG-I overexpressing HEK-Lucia reporter cells, we proceeded to investigate whether our template-specific RIG-I agonist precursor could induce activation of pro-apoptotic signaling in the B16-F10 melanoma cell line. B16-F10 melanoma cells are characterized by moderate expression levels of miR-21 (FIG. 3a) and have previously been employed to study intrinsic RIG-I signaling with cell death as an endpoint [Bek, S., Stritzke, F., Wintges, A., Nedelko, T., Bohmer, D. F. R., Fischer, J. C., Haas, T., Poeck, H., and Heidegger, S. (2019). Targeting intrinsic RIG-I signaling turns melanoma cells into type I interferon-releasing cellular antitumor vaccines. Oncoimmunology 8, e1570779].

IP-10/CXCL-10 is a member of the CXC chemokine family and induced by IFNs, especially IFN-γ. It plays a crucial role in the recruitment of activated T cells. To verify its expression associated with RIG-I activation, we assessed the IP-10 protein in the culture supernatant of B16-F10 cells after 48 h of treatment with increasing concentrations of RIGA-miR-21 and corresponding controls. FIG. 3b demonstrates a substantial induction of IP-10 in cells transfected with mature miR-21 mimic (300 or 600 ng/mL) and subsequently treated with RIGA-miR-21. In contrast, this effect was not observed in the control treatments or in the absence of the miR-21 mimic.

To align with the known mechanism of apoptosis induction via tumor-cell intrinsic RIG-I signaling, we initially assessed caspase-3/7 activation in B16-F10 melanoma cells treated with RIGA-miR-21 or its 5′-ppp-deficient equivalent (i.e., anti-miR-21). The results revealed a dose-dependent caspase-3/7 activation, with a more pronounced effect observed in the presence of a 5′-ppp (FIG. 3c, 30,000 cells). Subsequently, we transiently transfected the cells with miR-21 mimic (endogenous miR-21 sequence that is complementary to the agonist precursor sequence) prior to treatments with the constructs and evaluated caspase-3/7 activation. The data showed a miR-21 dose-dependent increase in caspase-3/7 activation, which was significantly higher in cells treated with RIGA-miR-21 compared to its 5′-ppp-deficient counterpart (FIG. 3d, 10,000 cells). These findings demonstrated that activation of RIG-I using the template-directed approach was capable of initiating caspase-3/7 signaling and priming the cells for apoptosis.

In our subsequent analysis, we investigated the expression levels of RIG-I to determine whether, in addition to RIG-I activation, there was evidence of RIG-I upregulation in B16-F10 cells treated with RIGA-miR-21. Low basal levels of RIG-I were detected in B16-F10 cells, which were upregulated in the cells treated with both RIGA-miR-21 and miR-21 or ppp-dsRNA control, but not miR-21 or RIGA-miR-21 alone (FIG. 3e).

We then assessed the phosphorylation status of NF-κB, a key regulator of the inflammatory process in the nuclear factor kappa B (NF-κB) pathway. One of the mechanisms of immune activation by RIG-I agonism involves the activation of NF-κB to regulate the expression of proinflammatory and proapoptotic genes [Ramos, H. J., and Gale, M., Jr. (2011). RIG-I like receptors and their signaling crosstalk in the regulation of antiviral immunity. Curr Opin Virol 1, 167-176]. In our studies, we analyzed the phosphorylation of the NF-κB subunit p65 at 5536 to measure NF-κB transactivation. We observed strong phospho-P65 reactivity in lysates from B16-F10 cells treated with RIGA-miR-21, which was further amplified if the cells were also transfected with a miR-21 mimic (FIG. 3f). This effect was not associated with increased expression of p65, indicating that it specifically reflected target phosphorylation. This observation provided further support for effective template-dependent immune stimulation by RIGA-miR-21. The impact by 5′-ppp-dsRNA control or miR-21 mimic confounded by the observation that LyoVec alone could cause significant p65 phosphorylation.

In summary, RIG-I activation was observed in BF16 cells as indicated by low, but detectable levels of Caspase-3/7 activation, NF-κB phosphorylation and reduced cell viability although not reflected by IP-10 and RIG-I, which also rely on the supplementation of miR-21 mimic.

### Example 5—Nanoparticle Formulation of RIGA-miR-21 Targeted for Systemic Delivery

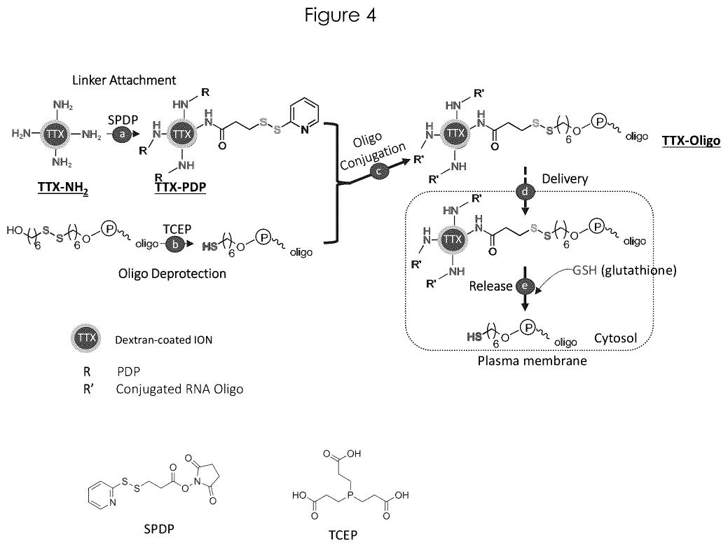

With the demonstrated efficacy of RIGA-miR-21 in activating RIG-I signaling in melanoma cells, particularly in combination with miR-21 mimic, our next objective was to develop a nanoparticle formulation suitable for systemic delivery. Aminated dextran-coated iron oxide nanoparticles were synthesized prior to this study through co-precipitation of iron oxide from Fe(II) and Fe(III) chloride solutions under alkali conditions in the presence of dextran. The dextran shell was stabilized via cross-linking and amination using ammonia. To enable the conjugation of oligos, a disulfide linker was incorporated into the aminated iron oxide nanoparticles (TTX-NH 2) leading to the formation of TTX-PDP. The thiol-modified oligos used in the conjugation process are typically delivered in their oxidized form and can be easily activated through reduction using TCEP. The deprotected oligos were subsequently conjugated to the nanoparticles through thiol-disulfide exchange reactions to produce the final product (TTX-oligo). This design allows the payload to be delivered via the nanoparticles into the cells and released in the cytosol upon exposure to the cytosolic glutathione (GSH) (FIG. 4).

### Example 6—the Nanoparticle Formulation of RIGA-miR-21 can be Successfully Delivered and Effectively Agonizes RIG-I in the Reporter Cell Line

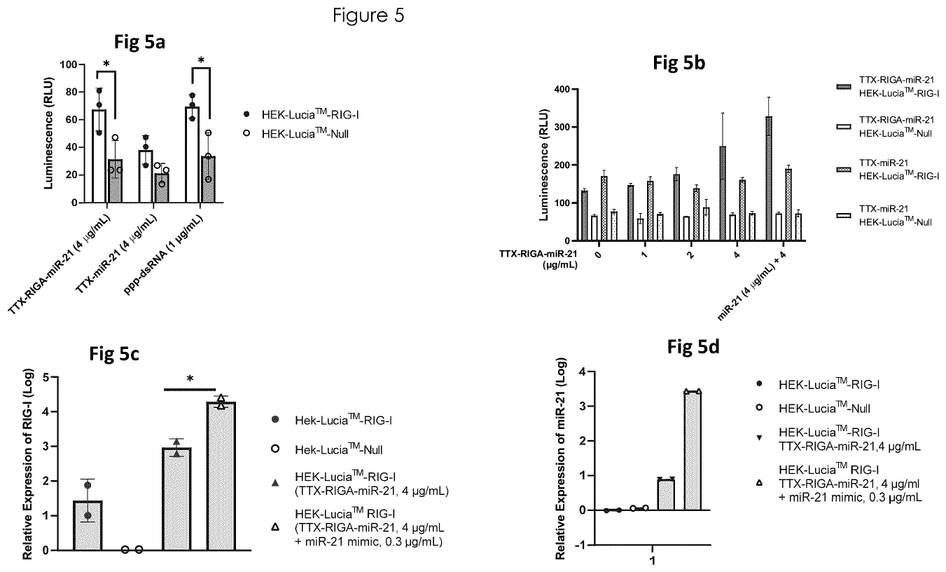

In line with the previous studies utilizing RIGA-miR-21, we proceeded to evaluate the efficacy of TTX-RIGA-miR-21 in reporter cell lines. HEK-Lucia™ RIG-I and HEK-Lucia™ Null control cells were treated with TTX-RIGA-miR-21 or controls, TTX-miR-21 or 5′-ppp-dsRNA (transfected with LyoVec). RIG-I activation was observed in HEK-Lucia™ RIG-I cells treated with TTX-RIGA-miR-21 (4 μg/mL) or the ppp-dsRNA control. However, the ppp-deficient TTX-miR-21 failed to induce detectable RIG-I activation (FIG. 5a) A subsequent dosage study ranging from 1 to 4 μg/mL of TTX-RIGA-miR-21 was conducted (FIG. 5b). Basal luciferase expression was observed in HEK-Lucia™ RIG-I compared to HEK-Lucia™ Null, which did not exhibit responsiveness to lower concentrations of TTX-RIGA-miR-21 treatments (1 or 2 μg/mL). Consistent with the findings in FIG. 5a, the luciferase activity levels were significantly elevated with TTX-RIGA-miR-21 at 4 μg/mL, and further augmented when the cells were pre-treated with miR-21 mimic.

To investigate the expression of RIG-I, we performed reverse transcription polymerase chain reaction (RT-PCR) analysis. The mRNA levels of RIG-I were evaluated in both HEK-Lucia™ RIG-I and HEK-Lucia™ Null control cells. Consistent with the Western blot results (FIG. 2b), we confirmed the presence of basal level RIG-I expression in HEK-Lucia™ RIG-I cells. This expression level was slightly higher compared to the HEK-Lucia™ Null control cells (FIG. 5c). Treatment with TTX-RIGA-miR-21 led to significant upregulation the mRNA levels of RIG-I in HEK-Lucia™ RIG-I, which were further enhanced if the cells were pre-treated with miR-21 mimic.

We also assessed the expression levels of miR-21 in HEK-Lucia™ RIG-I cells using RT-PCR analysis in response to TTX-RIGA-miR-21 treatment. We confirmed the basal level expression of miR-21 observed earlier in HEK-Lucia™ RIG-I cells (FIG. 2a), which, however, were significantly upregulated upon treatment with TTX-RIGA-miR-21 (4 μg/mL) in HEK-Lucia™ RIG-I cells. When exogenous miR-21 mimic was supplied in addition to TTX-RIGA-miR-21, there was a further increase in miR-21 expression levels (FIG. 5d), which likely summed up the contributions of upregulated miR-21 and exogenous miR-21 mimic.

In summary, like RIGA-miR-21 transfected with LyoVec, TTX-RIGA-miR-21 was able to induce template-dependent RIG-I activation in HEK293 reporter cells, which was indirectly supported by the positive feed-back upregulation of both RIG-I and the miR-21 template. The results demonstrated that RIGA-miR-21 can be successfully delivered into the cells via the TTX platform.

### Example 7—TTX-RIGA-miR-21 Effectively Agonizes RIG-I and Induces Apoptosis in Melanoma Cells

Building upon the successful delivery of TTX-RIGA-miR-21 and its activation of RIG-I signaling in HEK-Lucia reporter cells, we extended our investigations to the B16-F10 melanoma cell line, which has been shown to express moderate levels of miR-21 (FIG. 3a). This cell line provides a relevant model to assess the efficacy of TTX-RIGA-miR-21 in a melanoma context and evaluate its potential as a therapeutic approach for targeting miR-21 in cancer cells.

IFN-β, IP-10, and RIG-I

The induction of type I interferon (IFN) is a crucial response of the innate immune system upon sensing nucleic acids. In our initial study using RT-PCR, we observed no detectable change in IFN-r3 mRNA expression in cells treated with TTX-RIGA-miR-21 (1-4 μg/mL), but significant elevation of IFN-r3 expression in the cells that received dual treatment of TTX-RIGA-miR-21 and miR-21 mimic, or 5′-ppp-dsRNA, which served as a positive control (FIG. 6a). Further analysis of IFNβ protein using ELISA confirmed strict dependence of IFNβ upregulation on the exogenous miR-21 template (FIG. 6b). Neither TTX-RIGA-miR-21 nor miR-21 mimic alone was able to detectable change of IFN-0.

Similar observations were made for IP-10, which was only significantly elevated in the cells that had received dual treatment of TTX-RIGA-miR-21 and miR-21 mimic, or 5′-ppp-dsRNA (FIG. 6c, 6d). Neither TTX-RIGA-miR-21 nor miR-21 mimic alone produced delectable IP-10 response.

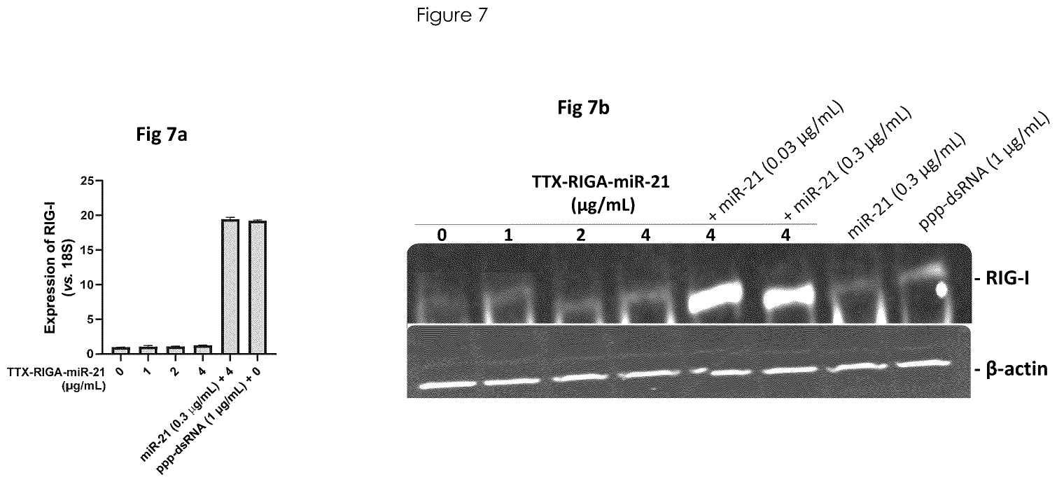

To investigate whether RIG-I activation in B16-F10 cells treated with TTX-RIGA-miR-21 is accompanied by RIG-I upregulation, we assessed RIG-I expression at the mRNA level using RT-PCR and at the protein level using SDS-PAGE. Both assays showed that the expression of RIG-I remained unchanged when the cells were challenged with TTX-RIGA-miR-21 alone (1-4 μg/mL), but significantly elevated when the cells were also supplied with mR-21 mimic prior to TTX-RIGA or treated with ppp-dsRNA (FIG. 7a, 7b). The results were consistent with the strict miR-21 dependence observed for RIGA-miR-21, transfected with LyoVec.

Caspase 3/7, TRAIL and Cell Viability

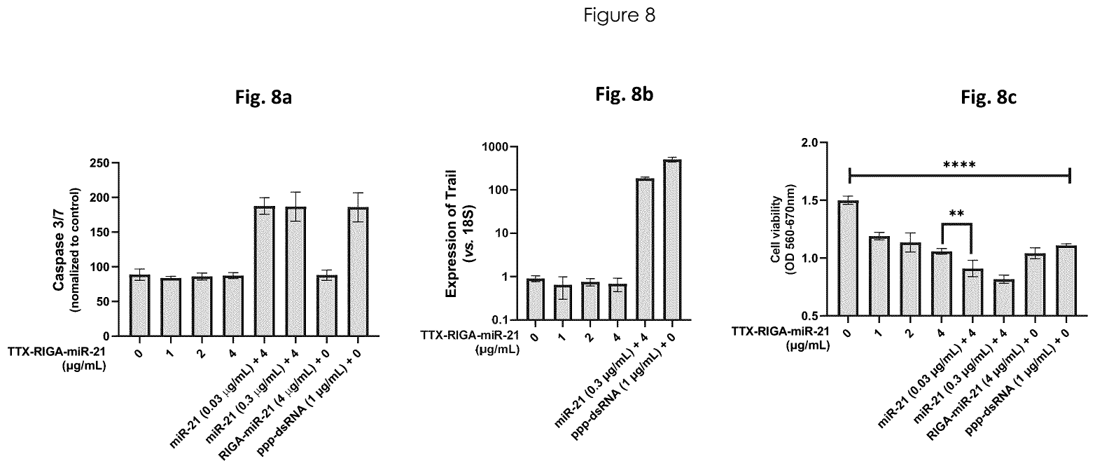

To evaluate the induction of apoptosis through tumor cell-intrinsic RIG-I signaling, we examined caspase-3/7 activation in B16-F10 cells by TTX-RIGA-miR-21 (FIG. 8a). Caspase-3/7 remained unchanged when the cells were challenged with TTX-RIGA-miR-21 or miR-21 mimic alone but was significantly boosted with the dual treatment. Compared with low levels of caspase-3/7 activation observed for RIGA-miR-21/LyoVec, the lack of effect by TTX-RIGA-miR-21 on caspase-3/7 activation could be attributed to the reduction of the cells used in the current assay.

Next, we investigated the activation of the extrinsic apoptotic pathway by examining the levels of TNF-related apoptosis-inducing ligand (TRAIL), which is a biomarker for the activation of RIG-I signaling pathway and associated with the initiation of the extrinsic apoptotic pathway. TRAIL has been demonstrated to selectively induce apoptotic cell death in various tumor cells by binding to its death-inducing receptors. Similar to our observations for Caspase-3/7 activation, we only found that the secretion of TRAIL in cells treated with both TTX-RIGA-miR-21 and miR-21 mimic, not TTX-RIGA-miR-21 alone. Although the efficacy was not as pronounced as that of ppp-dsRNA, the combination of TTX-RIGA-miR-21 with miR-21 mimic significantly upregulated the expression of TRAIL (FIG. 8b).

We then conducted a cell viability assay to evaluate the overall impact of RIG-I activation on melanoma cells by TTX-RIGA-miR-21. In contrast to the lack of detectable effect on most of the biomarkers evaluated above in the absence of miR-21 mimic, treatment with TTX-RIGA-miR-21 alone resulted in dose-dependent cell death, even at a low dose of 1 μg/mL. This effect was further enhanced as miR-21 mimic was provided in combination with TTX-RIGA-miR-21 (FIG. 8c), confirming its template-dependence nature of cell death. Notably, TTX-RIGA-miR-21 at 4 μg/mL alone demonstrated comparable efficacy to the LyoVec-delivered RIGA-miR-21 plus miR-21 mimic or the 5′-ppp-dsRNA control in inducing cell death.

The cell viability assay provides a comprehensive assessment of the effect of RIG-I activation on melanoma cells reflecting the accumulative effects of the activation of downstream pathways and processes that contribute to cell survival or death, which, otherwise, may not be detectable individually.

The observed dose-dependent reduction in cell viability with TTX-RIGA-miR-21 treatment indicates that the template-directed approach to RIG-I activation using a single agonist precursor has the potential to induce cell death specifically in melanoma and other cancer cells.

### Example 8—TTX-RIGA-miR-21 Effectively Reduces Tumor Burden of the Primary Tumor, Induce Long-Term Immunity and Dramatically Reduces the Secondary Tumor Burden

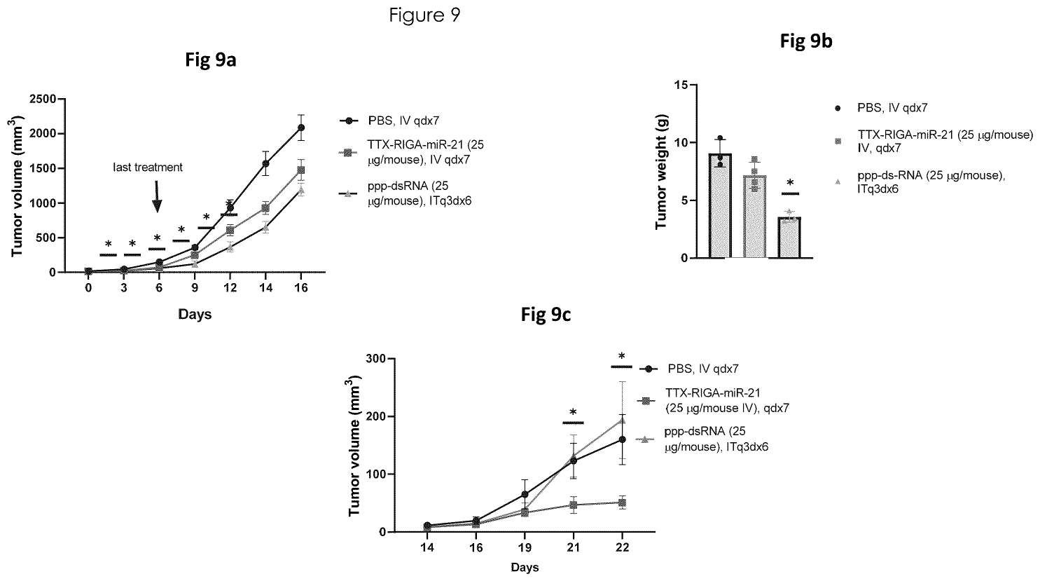

In an in vivo mouse model, we investigated the ability of TTX-RIGA-miR-21 to inhibit tumor development. Tumor cells were transplanted into the left flank of the mice and different treatments were administered at indicated time points. TTX-RIGA-miR-21 and PBS were delivered intravenously while free 5′-ppp-dsRNA was delivered intratumorally. The growth of the tumors was monitored by caliber measurements, and the weight of the primary tumors was measured upon sacrificing the mice. Both intravenously delivered TTX-RIGA-miR-21 and intratumorally delivered ppp-dsRNA significantly inhibited tumor growth compared to the PBS control (FIG. 9a). The observation with TTX-RIGA-miR-21 was noteworthy considering the dilution effect and the multiple obstacles the nanodrug had to overcome to reach the target site. Although the impact of TTX-RIGA-miR-21 on tumor weight was less impressive compared to the positive control, it still showed a trend towards lower tumor weight compared to the PBS control (FIG. 9b).

Importantly, we investigated whether TTX-RIGA-miR-21 could induce memory immunity to inhibit the development of secondary tumors. Tumor cells were implanted on the opposite side of primary tumor on the mice on Day 14, and the secondary tumor growth was monitored (FIG. 9c). Up to day 19, there was no difference observed between the study arms. However, as the tumors continued to grow, TTX-RIGA-miR-21 demonstrated a remarkable capability to inhibit tumor growth on days 21 and 22, while the response to 5′ppp-ds-RNA remained no difference from the PBS control. These findings clearly demonstrate that systemically delivered TTX-RIGA-miR-21, as a RIG-I agonist precursor, was able to induce memory immunity and inhibit the development of secondary tumors.

Provided herein are RIG-I agonist precursors, agonists thereof and methods for treating and/or inhibiting primary tumor growth and secondary tumor growth. In certain embodiments are methods and compositions for treating and preventing growth and/or formation of secondary tumors.

### Example 9. Synthesis of (Non) Radiolabeled TTX-Oligo (e.g., TTX-Oligo-^{nat/64}Cu²⁺)

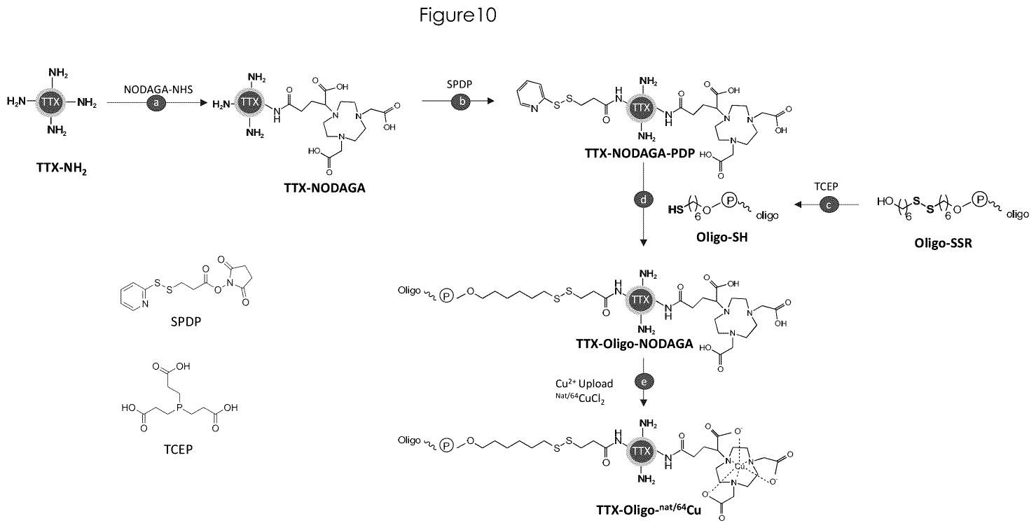

The synthesis of (non) radiolabeled TTX-Oligo from TTX-NH 2 platform is illustrated in FIG. 10 involving the following steps: a) Coupling reaction between TTX-NH₂ and NODAGA-NHS to form TTX-NODAGA. b) Functionalization with the heterobifunctional linker SPDP to form TTX-NODAGA-PDP. c) Deprotection of oligo leading to thiol oligo (Oligo-SH). d) Conjugation of thiol oligo via thiol disulfide exchange reaction to form TTX-Oligo-NODAGA. e) Complexation with ^{nat}CuCl₂ or ⁶⁴CuCl₂ leading to TTX Oligo-^{nat/64}Cu²⁺. The entire procedure is described below in detail.

A. Synthesis of TTX-NODAGA-PDP

To synthesize NPs dually functionalized for radiolabeling and oligo conjugation, TTX-NH₂ will be sequentially treated with NODAGA-NHS to introduce the chelator for Cu²⁺ loading, and SPDP, a heterobifunctional linker, to introduce the redox-sensitive disulfide linker for oligo conjugation through thiol-disulfide exchange reactions. TTX-NH₂ solution will be diluted to ≤10 mg Fe/mL with PBS, and chilled on ice. NODAGA-NHS will be dissolved in anhydrous DMSO (¼ volume of TTX-NH 2 solution, ˜0.0625 mg/mg iron). NODAGA-NHS solution will be added to TTX-NH₂ solution and rapidly mixed. This reaction mixture will be incubated overnight on a shaker in the refrigerator at 2-8° C. SPDP solution will be prepared by dissolving the powder in DMSO (¼ volume of TTX-NH 2 solution, ˜0.325 mg/mg iron). The freshly prepared SPDP/DMSO solution will then be added to the TTX-NODAGA solution and mixed well by pipetting or stirring, and the mixture is then incubated overnight on a shaker in the refrigerator maintained at 2-8° C. TTX-NODAGA-PDP reaction mixture will then be diluted with 4 volumes of PBS, purified and buffer exchanged to PBS using Amicon Ultra-15 mL filters (30 or 100 kDa). The final product will be sterile filtered and stored at 2-8° C. in the refrigerator. Characterizations will include iron content, size and size distribution, and the number of PDP ligands following appropriate protocols. To quantify the number of NODAGA per NP, the number of amines per NP will be subtracted from the number of amines per NP after conjugation with NODAGA.

B. Synthesis of TTX-Oligo-NODAGA

B1. Exemplary Procedure for Deprotecting Disulfide Protected Oligonucleotides

Disulfide protected oligos will be prepared as 1 mM solution in nuclease-free water (NFW). Also, separately prepared will be 3% TCEP (w/v) in NFW. Fifty μL of TCEP solution will be added to 0.5 mL of oligo solution and incubate at room temperature for 30 mins. Resulting thiol-oligos will be purified using ethanol/ammonium acetate precipitation method (briefly, addition of 250 μL of 9.5 M ammonium acetate to the oligo mixture, followed by 1150 μL of cold ethanol). White cloudy oligo precipitation will be visible. This cloudy solution will be kept at −80° C. for 1 h followed by centrifugation of the mixture for 15 min at 4° C., 20,000×g. A white oligo pellet will form at the bottom of the tube. Discard the supernatant and wash the pellet with 1 mL of 100% ethanol and 0.5 mL of 70% ethanol (prepared from 100% ethanol with NFW) sequentially. Further, the oligo pellet will be dried using a speed vacuum concentrator and re-suspend the pellet in 0.25 mL of NFW, label it as thiol-oligo and store in refrigerator set at 2-8° C.

B2. Exemplary Procedure for Conjugating Oligonucleotide to Nanoparticles (TTX-NODAGA-PDP)

TTX-NODAGA-PDP will be mixed with the thiol-oligo at a molar ratio of 1:13. The resulting reaction mixture will be incubated on a rotator in the cold room for 24 h. This will be followed by purification to concentrate the nanodrug solution using Amicon Ultra 15 mL centrifugation filters (MWCO 30 kDa) using similar filtration procedure described above. The final product TTX-oligo-NODAGA, will be stored in the 2-8° C. refrigerator with gentle rotation.

C. Synthesis and Characterization of TTX-Oligo-⁶⁴Cu

The procedure by Le Fur et al. will be followed with modifications [Le Fur M, Ross A, Pantazopoulos P, Rotile N, Zhou I, Caravan P, Medarova Z, Yoo B. Radiolabeling and PET-MRI microdosing of the experimental cancer therapeutic, MN-anti-miR10b, demonstrates delivery to metastatic lesions in a murine model of metastatic breast cancer. Cancer Nanotechnol. 2021; 12(1):16]. Briefly, copper chloride (⁶⁴CuCl₂) and TTX-Oligo-NODAGA solution will be pre-diluted into 0.2 M sodium citrate (pH 8.0) and then mixed at the desired ratio. The reaction will then be heated to 65-70° C. for 30 min in a heating block during which time the TTX-oligo-NODAGA precursor will chelate the ⁶⁴Cu²⁺ ion to give TTX-Oligo-Cu64 (TTX-Oligo-⁶⁴Cu). The radiochemical purity will be determined by radio iTLC (instant thin-layer chromatography). If the radiochemical purity is <90%, residual unreacted ⁶⁴Cu will be removed by centrifugal filtration.

The radiochemical purity of the product will be analyzed by iTLC (Agilent, iTLC-SG, Santa Clara, CA) with an EDTA solution as an eluent (50 mM, pH 5) using a radio-TLC imaging scanner (AR-2000, Eckert & Ziegler, Berlin, Germany). Optionally, radiochemical identity of the final solution of TTX-Oligo-′Cu can be confirmed by analytical HPLC (Agilent 1100 HPLC system, Santa Clara, CA) with a size exclusion column (TSK gel QC-PAK-300, isocratic, 100% sodium phosphate 0.1 M pH 7.4, 20 min) and a Carroll/Ramsey radioactivity detector with a silicon PIN photodiode and with UV detection at 254 nm.

Inductively Coupled Plasma Mass Spectrometry (ICP-MS) analysis (Agilent 8800-QQQ system, Santa Clara, CA) will be carried out to determine the concentrations of copper and iron. All samples will be prepared by weight. Calibration standards will be prepared by diluting certified copper and iron standards (1000 mg/L). Calibration curve will be obtained from 5 standard solutions in the range from 0.1 to 400 ppb. Lutetium (1 ppm) will be used as an external standard to ensure the proper introduction of the sample. The hydrodynamic diameter and Zeta-potential will be measured by a dynamic light scattering spectrometer (Zetasizer Nano, Malvern, UK) and the size of the iron oxide core will be determined by transmission electron microscopy (JEM 2100 TEM, Jeol, Tokyo, Japan).

D. Synthesis of non-radioactive TTX-Oligo-^{nat}Cu²⁺

Non-radioactive TTX-Oligo-^{nat}Cu²⁺ will be used as a control in various studies. TTX-Oligo-NODAGA solution and CuCl₂, both pre-diluted into 0.2 M sodium citrate (pH 8.0), will be mixed at the desired ratio (NODAGA:Cu ratio, e.g., 1:50). The reaction will then be heated to 65-70° C. for 30 min in a heating block during which time the TTX-oligo-NODAGA precursor chelates the Cu²⁺ to give TTX-Oligo-^{rat}Cu²⁺. The mixture will be diluted with 0.1 volume of 0.1 M EDTA (pH 7.4) to chelate free Cu²⁺ ions, followed by purification and concentration with ultrafiltration using nuclease-free PBS buffer as an eluent. The Cu²⁺ load of the final TTX-Oligo-^{nat}Cu²⁺ product will be determined by ICP-MS.

### Example 10. Synthesis of Radioiodine-Labeled TTX-Oligo (e.g., TTX-Oligo-¹²⁵I)

Radiolabeled pharmaceuticals, including iodine-based radiopharmaceuticals, have gained significant attention in the field of chemistry and biomedicine. These compounds are used in clinical practice for both diagnostic and therapeutic purposes, primarily in the imaging of various diseases such as oncological, neurological, cardiovascular, gastrointestinal, and endocrine conditions. The use of radiolabeled compounds for diagnostics is advantageous due to their easy detection and the ability to determine very small amounts. They allow for noninvasive visualization of anatomical and physiological manifestations of diseases. As therapeutic agents, radiopharmaceuticals can be used to selectively target and destroy cancer cells, leading to their death.

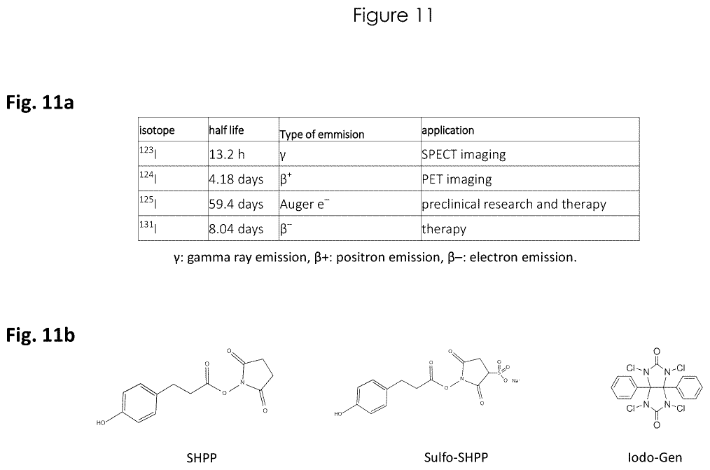

Radioactive halogen-containing compounds, particularly iodine isotopes, play a significant role in nuclear medicine due to their favorable chemical properties and nuclear decay characteristics. Iodine radioisotopes, such as iodine-125 and iodine-131, are widely utilized for labeling both low-molecular-weight compounds and larger biomolecules like peptides and nucleic acids. The longer half-life of iodine isotopes compared to other commonly used radioisotopes (e.g., carbon-11 and fluorine-18) offers advantages in terms of diverse synthetic methods for radioiodination (FIG. 11a). Furthermore, iodine-containing compounds tend to have increased lipophilicity, which can enhance their pharmacological and pharmacokinetic properties. Radioactive iodine itself has a tendency to accumulate in the thyroid gland, stomach, and salivary glands. To deliver it to other target organs, radioactive iodine is often combined with molecules that specifically bind to those tissues. Iodine-based radiopharmaceuticals have proven to be valuable tools in nuclear medicine. Iodine radionuclide therapy is employed for the treatment of various cancers, including thyroid cancer, pheochromocytomas and paragangliomas, and non-Hodgkin lymphomas. In summary, their optimal half-life, diverse synthetic methods, and ability to accumulate in specific tissues make iodine radionuclides useful for both diagnostic imaging and targeted therapy in various diseases, particularly in the field of oncology.

Iodination at tyrosine residues has been widely used to label proteins or peptides in various applications. Iodinated N-hydroxysuccinimide ester of 3-(4-hydroxyphenyl) propionic acid, also known as Bolton-Hunter reagent or SHPP, is successfully used to iodinize proteins that lack tyrosine residues. In this method, the protein is covalently coupled with the acylating agent, where the acylating agent reacts with protein side chains at lysine residues. This method is more suitable for proteins with few or no tyrosine residues, and also suitable for proteins susceptible to oxidation. This method has many applications including target labeling of cytosolic proteins, membrane proteins, viruses, cell lysates and compounds like Gentamicin.

Reagents typically used for iodine radiolabeling of proteins/peptides following the Bolton-Hunter procedure are listed in FIG. 11b, including Bolton-Hunter Reagent (SHPP) or its water-soluble Bolton-Hunter Reagent version (Sulfo-SHPP), and lodo-Gen. The iodination reaction in Bolton-Hunter procedure using lodo-Gen involves the following steps: lodo-Gen is dissolved in an organic solvent, such as chloroform or methanol, to create a stock solution. The reaction mixture is incubated, allowing the lodo-Gen to coat the surface of the reaction vessel, followed by evaporation of the solvents. The protein or peptide of interest is mixed with the lodo-Gen stock solution. A solution containing radioactive iodine (usually in the form of Na[¹²⁵I] or Na[¹³¹I]) is added to the reaction mixture. The iodine reacts with the tyrosine or phenolic groups on the protein, leading to the incorporation of radioactive iodine into the molecule. The reaction is quenched, typically by the addition of a reducing agent such as sodium metabisulfite or sodium thiosulfate. This method enables the specific iodination of tyrosine residues in proteins, including those present in cell membranes. It can also be used to label phenolic groups on crosslinkers or other protein modification reagents.

### Experimental

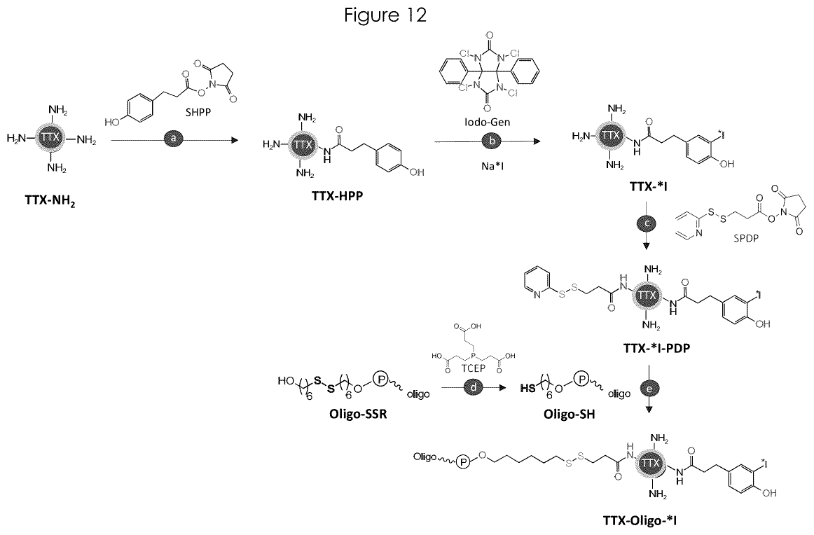

In the current application, the Bolton-Hunter procedure will be adapted for iodine radio-labeling of the TTX platforms. The entire procedure is outlined in FIG. 12. Here is a step-by-step overview of the process:

Functionalization of TTX-NH 2 platform for radio-labeling: TTX-NH 2 NPs will be treated with a controlled amount of SHPP or Sulfo-SHPP (sulfosuccinimidyl 3-(4-hydroxyphenyl)propionate) to introduce phenolic groups. It is important to ensure that only a fraction of the amine groups on the TTX-NH₂ NPs is utilized in this reaction. After the treatment, the product will be purified using ultrafiltration. This process helps remove excess reagents and byproducts from the reaction mixture, leaving behind the desired product (TTX-HPP).

Iodine Radio-labeling: TTX-HPP product will be subjected to iodine radio-labeling. This will be achieved using Iodo-Gen and Na[125I] or Na[131I], which are sources of radioactive iodine isotopes. Following the radio-labeling step, the radio-labeled TTX-*I product will undergo another round of ultrafiltration to purify the sample and remove any unreacted radioiodine or other impurities.

Functionalization of TTX-*I with SPDP: The purified radio-labeled TTX-*I will be functionalized with SPDP (N-succinimidyl 3-(2-pyridyldithio)propionate). This step involves attaching the PDP groups to the unused amine groups, enabling subsequent conjugation with oligos. The product obtained after functionalization with SPDP (TTX-*I-PDP) will be purified once again to remove excess reagents and impurities.

Oligo Deprotection: Activation of the protected thiol-modified oligos will be performed by reduction with TCEP and purification by ethanol/ammonium acetate precipitation.

Oligo Conjugation: The purified radio-labeled TTX-*I-PDP will be conjugated to oligos. This step involves the attachment of thiol-oligos to the TTX-*I-PDP, forming the final product (TTX-Oligo-*I), which will be subsequently purified using ultrafiltration.

Cold Control—TTX-Oligo-I: As a control, a “cold” control sample may be synthesized in parallel with the TTX-Oligo-*I conjugate. This control sample will be prepared using non-radioactive NaI instead of the radioactive Na[125I] or Na[131I] used for radio-labeling. The cold control allows for comparisons and assessments specific to the effects of radioactivity.

Quality Control (QC): The final product, the TTX-Oligo-*I, will undergo standard quality control procedures to ensure its integrity, purity, and functionality.

In Vitro Studies: In vitro studies such cell viability assay may be conducted to evaluate the performance of the TTX-Oligo-*I product.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1. A nanoparticle comprising:
a core-shell structure, wherein the shell comprises a RIG-1 agonist precursor comprising a single-stranded 5′ uncapped triphosphate antisense oligonucleotide having a sequence complementary to an endogenous miRNA; and optionally a radiolabel.

2. The nanoparticle of claim 1, wherein the shell further comprises a single stranded oligonucleotide sequence complementary to the single-stranded 5′ uncapped triphosphate antisense oligonucleotide.

3. The nanoparticle of claim 1, wherein the miRNA is selected from the group consisting of miR10b, miR17, miR18a, miR18b, miR19b, miR21, miR26a, miR29a, miR92a-1, miR92a-2, miR155, miR210, and miR221.

4-9. (canceled)

10. The nanoparticle of claim 1, wherein the shell comprises an aminated polysaccharide coating wherein the polysaccharide is selected from the group consisting of dextran, alginate, chitosan, chitin, cellulose, hyaluronic acid (HA), amylose, amylopectin, carrageenan, and a polysaccharide polymer consisting of maltotriose units (Pullulan).

11-12. (canceled)

13. The nanoparticle of claim 1, wherein the core comprises a polymer, a metal or a metal ion.

14-16. (canceled)

17. The nanoparticle of claim 1, further comprising a radiolabel or dye.

18. (canceled)

19. (canceled)

20. The nanoparticle of claim 17, wherein the radiolabel is selected from copper-64 (Cu-64), copper-67 (Cu-67), F-18, yttrium-90 (Y-90), scandium-44 (SC-44), cobalt-55 (co-niobium-90 (Nb-90), rhenium-186 (Re-186), rhenium-188 (Re-188), terbium-161 (Tb-161), lutetium-177 (Lu-177), bismuth-231 (Bi-213), lead-212 (Pb-212), actinium-225 (Ac-225), zirconium-89 (Zr), or any combination thereof.

21-25. (canceled)

26. A pharmaceutical formulation for slowing growth of tumors in a subject comprising an effective amount of the nanoparticle of claim 1.

27. The pharmaceutical formulation of claim 26, further comprising at least one pharmaceutically acceptable carrier or diluent.

28. The pharmaceutical formulation of claim 26, formulated into a dosage form that is an injectable, a tablet, a lyophilized powder, a suspension, or any combination thereof.

29-38. (canceled)

39. A method for slowing growth of tumors in a subject in need thereof comprising administering the pharmaceutical formulation of claim 26.

40. A method for treatment of tumors in a subject in need thereof comprising administering the pharmaceutical formulation of claim 26.

41. (canceled)

42. The method of claim 40, wherein the tumor is a secondary tumor.

43-46. (canceled)

47. The method of 40, further comprising administering additional supportive or adjunctive therapy.

48-114. (canceled)

115. A method of generating a localized immune response comprising:
administering to the subject a therapeutically effective amount of a nanoparticle comprising
a nanoparticle core;
a radiolabel; and
a single-stranded 5′ uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment thereby generating a localized immune response.

116. (canceled)

117. A method of detecting, diagnosing, and/or monitoring treatment of a solid tumor in a subject, the method comprising:
administering to the subject a nanoparticle comprising
a nanoparticle core;
a radiolabel; and
a single-stranded 5′ uncapped triphosphate or biphosphate modified RNA oligonucleotide that is linked to the nanoparticle core, wherein said oligonucleotide is complementary to a miRNA which is highly expressed in a solid tumor or solid tumor microenvironment in comparison to a non-solid tumor or non-solid tumor microenvironment thereby generating a localized immune response.

118-137. (canceled)

138. The method of claim 115, wherein the miRNA is selected from the group consisting of miR10b, miR17, miR18a, miR18b, miR19b, miR21, miR26a, miR29a, miR92a-1, miR92a-2, miR155, miR210, and miR221.

139-141. (canceled)

142. The method of claim 138, wherein the modified RNA oligonucleotide is capable of forming a duplex with the said miRNA and wherein the duplex activates RIG-I.

143. (canceled)

144. (canceled)

145. The method of claim 142, wherein the RIG-I activation is at least 5%, 10%, 15% or 20% greater than activation by a corresponding unmodified monophosphate RNA oligonucleotide.

146-173. (canceled)

174. The method of claim 115, wherein the nanoparticle radiosensitizes the solid tumor.

175-205. (canceled)


SEQUENCE LISTING (48 sequences)

SEQ ID NO: 1 - RNA, 23 nt, Homo sapiens
  source 1..23: mol_type = genomic RNA
cacaaattcggttctacagggta

SEQ ID NO: 2 - RNA, 23 nt, Homo sapiens
  source 1..23: mol_type = genomic RNA
ctacctgcactgtaagcactttg

SEQ ID NO: 3 - RNA, 23 nt, Homo sapiens
  source 1..23: mol_type = genomic RNA
ctatctgcactagatgcacctta

SEQ ID NO: 4 - RNA, 23 nt, Homo sapiens
  source 1..23: mol_type = genomic RNA
ctaactgcactagatgcacctta

SEQ ID NO: 5 - RNA, 23 nt, Homo sapiens
  source 1..23: mol_type = genomic RNA
gctggatgcaaacctgcaaaact

SEQ ID NO: 6 - RNA, 22 nt, synthetic construct
  source 1..22: mol_type = other RNA
  misc_difference 1: note = Nucleotide may be 5'-triphosphate modified
  modified_base 22: mod_base = OTHER; note = 3' disulfide bond modified nucleotide
tcaacatcagtctgataagcta

SEQ ID NO: 7 - RNA, 23 nt, Homo sapiens
  source 1..23: mol_type = genomic RNA
agcctatcctggatttacttgaa

SEQ ID NO: 8 - RNA, 22 nt, Homo sapiens
  source 1..22: mol_type = genomic RNA
ctgaacaccaaaagaaatcagt

SEQ ID NO: 9 - RNA, 23 nt, Homo sapiens
  source 1..23: mol_type = genomic RNA
agcattgcaaccgatcccaacct

SEQ ID NO: 10 - RNA, 22 nt, Homo sapiens
  source 1..22: mol_type = genomic RNA
gtaatgcaacaaatccccaccc

SEQ ID NO: 11 - RNA, 24 nt, Homo sapiens
  source 1..24: mol_type = genomic RNA
aacccctatcacgattagcattaa

SEQ ID NO: 12 - RNA, 22 nt, Homo sapiens
  source 1..22: mol_type = genomic RNA
cagtgtgcggtgggcaggggct

SEQ ID NO: 13 - RNA, 22 nt, Homo sapiens
  source 1..22: mol_type = genomic RNA
aaatctacattgtatgccaggt

SEQ ID NO: 14 - RNA, 23 nt, Homo sapiens
  source 1..23: mol_type = genomic RNA
taccctgtagaaccgaatttgtg

SEQ ID NO: 15 - RNA, 23 nt, Homo sapiens
  source 1..23: mol_type = genomic RNA
caaagtgcttacagtgcaggtag

SEQ ID NO: 16 - RNA, 23 nt, Homo sapiens
  source 1..23: mol_type = genomic RNA
taaggtgcatctagtgcagatag

SEQ ID NO: 17 - RNA, 23 nt, Homo sapiens
  source 1..23: mol_type = genomic RNA
taaggtgcatctagtgcagttag

SEQ ID NO: 18 - RNA, 23 nt, Homo sapiens
  source 1..23: mol_type = genomic RNA
agttttgcaggtttgcatccagc

SEQ ID NO: 19 - RNA, 22 nt, Homo sapiens
  source 1..22: mol_type = genomic RNA
tagcttatcagactgatgttga

SEQ ID NO: 20 - RNA, 23 nt, Homo sapiens
  source 1..23: mol_type = genomic RNA
ttcaagtaaatccaggataggct

SEQ ID NO: 21 - RNA, 22 nt, Homo sapiens
  source 1..22: mol_type = genomic RNA
actgatttcttttggtgttcag

SEQ ID NO: 22 - RNA, 23 nt, Homo sapiens
  source 1..23: mol_type = genomic RNA
aggttgggatcggttgcaatgct

SEQ ID NO: 23 - RNA, 22 nt, Homo sapiens
  source 1..22: mol_type = genomic RNA
gggtggggatttgttgcattac

SEQ ID NO: 24 - RNA, 24 nt, Homo sapiens
  source 1..24: mol_type = genomic RNA
ttaatgctaatcgtgataggggtt

SEQ ID NO: 25 - RNA, 22 nt, Homo sapiens
  source 1..22: mol_type = genomic RNA
agcccctgcccaccgcacactg

SEQ ID NO: 26 - RNA, 22 nt, Homo sapiens
  source 1..22: mol_type = genomic RNA
acctggcatacaatgtagattt

SEQ ID NO: 27 - RNA, 23 nt, Homo sapiens
  source 1..23: mol_type = genomic RNA
tctttggttatctagctgtatga

SEQ ID NO: 28 - RNA, 22 nt, Homo sapiens
  source 1..22: mol_type = genomic RNA
tgaggtagtaggttgtatagtt

SEQ ID NO: 29 - RNA, 21 nt, Homo sapiens
  source 1..21: mol_type = genomic RNA
taaagtgctgacagtgcagat

SEQ ID NO: 30 - RNA, 22 nt, Homo sapiens
  source 1..22: mol_type = genomic RNA
gctcttttcacattgtgctact

SEQ ID NO: 31 - RNA, 22 nt, Homo sapiens
  source 1..22: mol_type = genomic RNA
tgagaactgaattccatgggtt

SEQ ID NO: 32 - RNA, 23 nt, Homo sapiens
  source 1..23: mol_type = genomic RNA
aacattcaacgctgtcggtgagt

SEQ ID NO: 33 - RNA, 23 nt, Homo sapiens
  source 1..23: mol_type = genomic RNA
aacattcattgctgtcggtgggt

SEQ ID NO: 34 - RNA, 21 nt, Homo sapiens
  source 1..21: mol_type = genomic RNA
cggcggggacggcgattggtc

SEQ ID NO: 35 - RNA, 22 nt, Homo sapiens
  source 1..22: mol_type = genomic RNA
catcttaccggacagtgctgga

SEQ ID NO: 36 - RNA, 22 nt, Homo sapiens
  source 1..22: mol_type = genomic RNA
ctgtgcgtgtgacagcggctga

SEQ ID NO: 37 - RNA, 22 nt, Homo sapiens
  source 1..22: mol_type = genomic RNA
tgcctgtctacacttgctgtgc

SEQ ID NO: 38 - RNA, 22 nt, Homo sapiens
  source 1..22: mol_type = genomic RNA
ctcagtagccagtgtagatcct

SEQ ID NO: 39 - RNA, 25 nt, Homo sapiens
  source 1..25: mol_type = genomic RNA
tcaagtcactagtggttccgtttag

SEQ ID NO: 40 - RNA, 23 nt, Homo sapiens
  source 1..23: mol_type = genomic RNA
tcaagagcaataacgaaaaatgt

SEQ ID NO: 41 - RNA, 22 nt, Homo sapiens
  source 1..22: mol_type = genomic RNA
ttataaagcaatgagactgatt

SEQ ID NO: 42 - RNA, 22 nt, Homo sapiens
  source 1..22: mol_type = genomic RNA
aactgtttgcagaggaaactga

SEQ ID NO: 43 - RNA, 23 nt, Homo sapiens
  source 1..23: mol_type = genomic RNA
tttcaagccagggggcgtttttc

SEQ ID NO: 44 - RNA, 22 nt, Homo sapiens
  source 1..22: mol_type = genomic RNA
agaccctggtctgcactctatc

SEQ ID NO: 45 - RNA, 23 nt, Homo sapiens
  source 1..23: mol_type = genomic RNA
caaagtgctgttcgtgcaggtag

SEQ ID NO: 46 - RNA, 22 nt, Homo sapiens
  source 1..22: mol_type = genomic RNA
acagattcgattctaggggaat

SEQ ID NO: 47 - RNA, 22 nt, Homo sapiens
  source 1..22: mol_type = genomic RNA
tcaacatcagtctgataagcta

SEQ ID NO: 48 - RNA, 22 nt, synthetic construct
  source 1..22: mol_type = other RNA
  modified_base 1: mod_base = OTHER; note = 5'-Triphosphate modified nucleotide
tcaacatcagtctgataagcta